(19) United States
(12) Patent Application Publication
(10) Pub. No.: US 2022/0062409 A1
(43) Pub. Date: Mar. 3, 2022

(54) HETEROLOGOUS PRIME BOOST VACCINE COMPOSITIONS AND METHODS

(71) Applicant: GLAXOSMITHKLINE BIOLOGICALS SA, Rixensart (BE)

(72) Inventors: Stefania CAPONE, Rome (IT); Nicolas Frederic DELAHAYE, Rockville, MD (US); Giulietta MARUGGI, Rockville, MD (US); Haifeng SONG, Rockville, MD (US)

(73) Assignee: GLAXOSMITHKLINE BIOLOGICALS SA, Rixensart (BE)

(21) Appl. No.: 17/413,203

(22) PCT Filed: Dec. 13, 2019
(86) PCT No.: PCT/IB2019/060766

§ 371 Date: Jun. 11, 2021

Related U.S. Application Data
(60) Provisional application No. 62/779,631, filed on Dec. 14, 2018.

(51) Int. Cl. A61K 39/245; A61K 39/21; A61K 39/205; A61P 31/14; A61P 31/18; A61P 31/22

(52) U.S. Cl. CPC A61K 39/245; A61K 39/21; A61K 39/205; A61K 2039/5256; A61P 31/18; A61P 31/22; A61P 31/14

(57) ABSTRACT

Simian adenoviral vectors and RNA molecules, each encoding an immunogen of interest, can be sequentially administered to provide potent and long-lasting immunity.

### FIELD OF THE INVENTION

The invention is in the field of preventing and treating infectious diseases. In particular, the invention relates to adenoviral vectors encoding disease related antigens and self-amplifying RNA molecules encoding disease related antigens. They can be combined in prime boost regimens to produce strong and sustained humoral and cellular immune responses.

### BACKGROUND OF THE INVENTION

Vaccination is one of the most effective methods for preventing infectious diseases. However, a single administration of an antigen is often not sufficient to confer optimal immunity and/or a long-lasting response. Approaches for establishing strong and lasting immunity to specific pathogens include repeated vaccination, i.e. boosting an immune response by administration of one or more further doses of antigen. Such further administrations may be performed with the same vaccine (homologous boosting) or with a different vaccine (heterologous boosting).

Adenoviral vectors have been demonstrated to provide prophylactic and therapeutic delivery platforms whereby a nucleic acid sequence encoding a prophylactic or therapeutic molecule is incorporated into the adenoviral genome and expressed when the adenoviral particle is administered to the treated subject. Most humans are exposed to and develop immunity to human adenoviruses. Thus, there is a demand for vectors which effectively deliver prophylactic or therapeutic molecules to a human subject while minimizing the effect of pre-existing immunity to human adenovirus serotypes. Simian adenoviruses are effective in this regard because humans have little or no pre-existing immunity to the simian viruses, yet these viruses are sufficiently closely related to human viruses to be effective in inducing immunity to delivered exogenous antigens.

RNA vaccines have been derived from genomic replicons that lack viral structural proteins and express a heterologous antigen in place of the viral structural proteins. These self-replicating, or self-amplifying, RNA molecules (SAM) can be produced either synthetically or in packaging cell lines that permit the expression of a single-round of infectious particles carrying RNAs encoding the vaccine antigen. RNA amplification in the cytoplasm then produces multiple copies of antigen-encoding mRNAs and creates double stranded RNA intermediates, which are known to be potent stimulators of innate immunity, i.e., the antigen non-specific defense mechanisms that deploy rapidly against almost any microbe. Synthetic replicon RNA vaccines have been demonstrated to achieve transient high levels of antigen production without the use of a live virus (Brito et al. (2015) Adv. Genetics 89:179).

A limitation of vaccination strategies is the induction of anti-vector immunity, leading to inefficient boosting upon re-administration of the same vector. This limitation can be partially offset by a suitable dosing interval, or overcome entirely by employing heterologous regimens that combine unrelated vectors. Various heterologous prime-boost regimens have been observed to improve the antigen-specific immune response after simian adenovector priming (Kardani et al. (2016) Vaccine 34:413).

A heterologous prime boost strategy has been demonstrated to improve the immunogenicity of alphavirus replicon vectored DNA in pigs by priming with alphavirus replicon DNA and boosting with a human adenovirus encoding a swine fever viral antigen (Zhao et al. (2009) Vet. Immunol. Immunopath. 131:158) and has been reported with respect to tumor antigens (Blair et al. (2018) Cancer Res. 78:724). Currently, one of the most explored prime boost combinations, as demonstrated in preclinical and some clinical settings, is adenoviral vector vaccine priming followed by recombinant Modified Vaccinia Ankara (MVA) virus boosting (Ewer et al. (2016) Curr. Opinion Immunol. 41:47). Although promising, MVA viral vector-based vaccine production for clinical applications presents challenges due to the complexities of manufacturing MVA. Thus, there remains a need in the art for heterologous prime boost regimens that provide robust immunogenicity without inducing anti-vector immunity.

### SUMMARY OF THE INVENTION

The invention provides potent prime-boost vaccination regimens in which RNA and adenoviral vaccine platforms are used to induce strong and long-lasting immunity to a range of antigens.

A first aspect of the invention provides a composition comprising or consisting of one or more of the constructs, vectors, RNA molecules or adenovirus molecules as described herein. Alternatively or additionally, the composition(s) comprise or consist of an immunologically effective amount of one or more of the constructs, vectors, RNA molecules or simian adenovirus molecules described herein.

In an embodiment, the invention provides a vaccine combination comprising a first composition comprising an immunologically effective amount of at least one adenovirus vector encoding at least one antigen and a second composition comprising an immunologically effective amount of at least one RNA molecule encoding at least one antigen wherein one of the compositions is a priming composition and the other composition is a boosting composition.

In an embodiment, the invention provides a vaccine combination comprising a first composition comprising an immunologically effective amount of at least one adenovirus vector encoding at least one antigen and a second composition comprising an immunologically effective amount of at least one self-amplifying RNA vector encoding at least one antigen wherein one of the compositions is a priming composition and the other composition is a boosting composition. In an embodiment, this self-amplifying RNA vector is produced synthetically. In another embodiment, this self-amplifying RNA vector is produced by in vitro translation.

In an embodiment, the invention provides a vaccine combination comprising a first composition comprising an immunologically effective amount of at least one simian adenovirus vector encoding at least one antigen and a second composition comprising an immunologically effective amount of at least one RNA molecule encoding at least one antigen wherein one of the compositions is a priming composition and the other composition is a boosting composition.

A second aspect of the invention provides a method of inducing an immune response in a mammal by administering a priming vaccine comprising an immunologically effective amount of an antigen encoded by either an adenoviral vector or an RNA molecule; and subsequently administering a booster vaccine comprising an immunologically effective amount of an antigen encoded by either an adenoviral vector or an RNA molecule, wherein if the priming vaccine is encoded by an adenoviral vector the booster vaccine is encoded by an RNA molecule and if the priming vaccine is encoded by an RNA molecule the booster vaccine is encoded by an adenoviral vector.

In an embodiment, the invention provides a method of inducing an immune response in a mammal with a priming vaccine comprising an immunologically effective amount of an antigen encoded by an adenoviral vector. In another embodiment, the invention provides a method of inducing an immune response in a mammal with a priming vaccine comprising an immunologically effective amount of an antigen encoded by an RNA molecule.

In an embodiment, the invention provides a method of inducing an immune response in a mammal with a priming vaccine comprising an immunologically effective amount of an antigen encoded by a simian adenoviral vector. In another embodiment, the invention provides a method of inducing an immune response in a mammal with a priming vaccine comprising an immunologically effective amount of an antigen encoded by an RNA molecule.

In an embodiment, the invention provides a method of inducing an immune response in a mammal with a priming vaccine comprising an immunologically effective amount of an antigen encoded by an adenoviral vector. In another embodiment, the invention provides a method of inducing an immune response in a mammal with a priming vaccine comprising an immunologically effective amount of an antigen encoded by a self-amplifying RNA vector.

In an embodiment, the invention provides a method of inducing an immune response in a mammal with a boosting vaccine comprising an immunologically effective amount of an antigen encoded by an adenoviral vector. In a yet further embodiment, the invention provides a method of inducing an immune response in a mammal with a boosting vaccine comprising an immunologically effective amount of an antigen encoded by an RNA molecule.

In an embodiment, the invention provides a method of inducing an immune response in a mammal with a boosting vaccine comprising an immunologically effective amount of an antigen encoded by a simian adenoviral vector. In a yet further embodiment, the invention provides a method of inducing an immune response in a mammal with a boosting vaccine comprising an immunologically effective amount of an antigen encoded by a self-amplifying RNA vector.

In an embodiment, the invention provides a method of inducing an immune response in a mammal with a boosting vaccine comprising an immunologically effective amount of an antigen encoded by an adenoviral vector. In a yet further embodiment, the invention provides a method of inducing an immune response in a mammal with a boosting vaccine comprising an immunologically effective amount of an antigen encoded by a self-amplifying RNA vector.

In an embodiment, the invention provides a method of inducing an immune response in a mammal with a priming vaccine comprising an immunologically effective amount of an antigen encoded by an adenoviral vector followed by a boosting vaccine comprising an immunologically effective amount of an antigen encoded by an RNA molecule. In another embodiment, the invention provides a method of inducing an immune response in a mammal with a priming vaccine comprising an immunologically effective amount of an antigen encoded by an RNA molecule followed by a boosting vaccine comprising an immunologically effective amount of an antigen encoded by an adenoviral vector.

In an embodiment, the invention provides a method of inducing an immune response in a mammal with a priming vaccine comprising an immunologically effective amount of one or more antigens of a pathogenic organism encoded by an adenoviral vector followed by a boosting vaccine comprising an immunologically effective amount of one or more antigens of the same pathogenic organism encoded by an RNA molecule. In an embodiment, the invention provides a method of inducing an immune response in a mammal with a priming vaccine comprising an immunologically effective amount of one or more antigens of a pathogenic organism encoded by an adenoviral vector followed by a boosting vaccine comprising an immunologically effective amount of one or more antigens of the same pathogenic organism encoded by an RNA molecule wherein the antigens have at least one non-identical epitope.

In another embodiment, the invention provides a method of inducing an immune response in a mammal with a priming vaccine comprising an immunologically effective amount of one or more antigens of a pathogenic organism encoded by an RNA molecule followed by a boosting vaccine comprising an immunologically effective amount of one or more antigens of the same pathogenic organism encoded by an adenoviral vector. In an embodiment, the invention provides a method of inducing an immune response in a mammal with a priming vaccine comprising an immunologically effective amount of one or more antigens of a pathogenic organism encoded by an RNA molecule followed by a boosting vaccine comprising an immunologically effective amount of one or more antigens of the same pathogenic organism encoded by an adenoviral vector wherein the antigens have at least one non-identical epitope.

In an embodiment, the one or more antigens from the same pathogenic organism are the same in the priming vaccine as in the boosting vaccine. In a yet further embodiment, at least one of the antigens from the same pathogenic organism are different in the priming vaccine and the boosting vaccine.

In any of the embodiments described herein, the immune response can be directed to an infectious organism, e.g., a virus, bacteria or fungus.

In an embodiment, the adenoviral vector is a simian adenoviral vector. In an embodiment, the simian adenoviral vector is a chimpanzee, bonobo, rhesus macaque, orangutan or gorilla vector. In an embodiment, the simian adenoviral vector is a chimpanzee vector. In an embodiment, the chimpanzee vector is AdY25, ChAd3, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, ChAd15, SadV41, ChAd157, ChAdOx1, ChAdOx2, sAd4287, sAd4310A, sAd4312, SAdV31 or SAdV-A1337. In an embodiment, the adenoviral vector is a bonobo vector. In an embodiment, the bonobo vector is PanAd1, PanAd2, PanAd3, Pan 5, Pan 6, Pan 7 or Pan 9.

In an embodiment of the adenoviral vector, the antigen is encoded in an expression cassette comprising a transgene and regulatory elements necessary for the translation, transcription and/or expression of the transgene in a host cell. In an embodiment, the transgene comprises one or more antigens. In an embodiment the transgene encodes a polypeptide antigen. In an embodiment, the transgene comprises a codon optimized antigen sequence or a codon pair optimized antigen sequence.

In an embodiment, at least one of the priming and boosting immunogenic compositions is administered by a route selected from buccal, inhalation, intramuscular, intranasal, intraperitoneal, intrathecal, intravenous, oral, rectal, sublingual, transdermal, vaginal or to the interstitial space of a tissue.

In an embodiment, least one of the priming and boosting immunogenic compositions comprises an adjuvant.

A third aspect of the invention provides a kit for a prime boost administration regimen according to any of the above embodiments comprising at least two vials, the first vial containing a vaccine for the priming administration and the second vial containing a vaccine for the boosting administration.

### DESCRIPTION OF THE DRAWINGS

FIG. 1A. Magnitude and kinetics of viral neutralizing antibody (VNA) titers to rabies RG antigen following a single dose. VNA titer is expressed as IU/ml. ChAd 10⁸ viral particles (vp) solid circles; ChAd 10⁷ vp solid squares; SAM/LNP 1.5 ug open circles; SAM/LNP 0.015 ug open squares; SAM/CNE 15 ug open triangles; SAM/CNE 1.5 ug open inverted triangles. Each dot represents average+/−SEM of titers from individual animals in the same group.

FIG. 1B. Magnitude and kinetics of CD8+ responses in blood following a single dose. CD8+ T cell responses to rabies RG antigen specific pentameric peptides is expressed as the percentage of positive cells. ChAd 10⁸ vp solid circles; ChAd 10⁷ vp solid squares; SAM/LNP 1.5 ug open circles; SAM/LNP 0.015 ug open squares; SAM/CNE 15 ug open triangles; SAM/CNE 1.5 ug open inverted triangles. Each dot represents mean+/−SEM of the percentage of RG-specific CD8+ T cells from individual mice.

FIG. 1C. T cell cytokine secretion induced in splenocytes at week 8 following a single dose. Data are expressed as IFN-γ Spot Forming Cells (SFC)/10⁶ splenocytes. Individual data points represent the total rabies RG protein response in each animal. Horizontal lines represent the group geometric mean.

FIG. 2A. Magnitude and kinetics of viral neutralizing antibody (VNA) titers following a priming dose and a homologous or heterologous boosting dose. Each dot represents antibody titer in an individual animal, and horizontal lines denote the group geometric mean. Rabies VNA titer for each of the seven prime boost regimens is expressed as IU/ml. Titers were measured 2, 4 and 8 weeks after the priming dose (w2, w4, w8) and 2, 4 and 8 weeks after the boosting dose (w2pb, w4pb, w8pb).

FIG. 2B. Magnitude and kinetics of CD8+ T cell responses in blood following a priming dose and a boosting dose of rabies RG antigen. CD8+ T cell responses to RG antigen-specific pentameric peptides is expressed as the percentage of positive cells. Individual data points represent the RG CD8+ response in each animal. Horizontal lines denote the group geometric mean.

FIG. 2C. T cell cytokine secretion induced in splenocytes at week 8 following a priming dose and a boosting dose of rabies RG antigen. Data are expressed as IFN-γ Spot Forming Cells (SFC)/10⁶ splenocytes. Individual data points represent the total RG antigen response in each animal. Horizontal lines represent the group geometric mean.

FIG. 3. Magnitude and kinetics of total antigen specific antibody titers following a single dose of a simian adenovirus encoding an HIV GAG transgene. HIV1 GAG antibody titer is expressed as the endpoint titer at days 14, 28, 42 and 56. ChAd-HIV-1 at doses of 3×10⁶ vp, 10⁷ vp and 10⁸ vp; and SAM-HIV1 with LNP at doses of 0.15 and 1.5 ug were compared to a saline control. Each dot represents the average ±SEM of the titers from individual animals in the same group.

FIG. 4A. Magnitude and kinetics of CD8+ responses in blood following a single dose of a simian adenovirus or SAMencoding an HIV GAG antigen. CD8+ T cell responses to HIV1 GAG antigen specific pentameric peptides is expressed as the percentage of positive cells. Individual data points represent the HIV1 GAG CD8+ response in each animal. Horizontal lines denote the group geometric mean.

FIG. 4B. CD4+ T cell response induced in splenocytes at week 8 following a single dose. Data are expressed as percentage of IFN-γ CD4+ positive cells. Individual data points represent HIV1 GAG protein response in each animal, obtained by combining the activity of the overlapping peptides. Horizontal lines represent the group geometric mean.

FIG. 4C. CD8+ T cell response induced in splenocytes at week 8 following a single dose. Data are expressed as percentage of IFN-γ CD8+ positive cells. Individual data points represent HIV1 GAG protein response in each animal, obtained by combining the activity to the overlapping peptides. Horizontal lines represent the group geometric mean.

FIG. 5. Magnitude and kinetics of HIV1 GAG-specific IgG titers following a priming dose and a boosting dose. Titers are expressed as endpoint titers and shown at days 15, 29, 43, 57 (day of boost) 71, 147 and 241.

FIG. 6A. Magnitude and kinetics of CD8+ responses in blood following a priming dose of a simian adenovirus or SAM encoding an HIV GAG antigen and a homologous or heterologous boosting dose. CD8+ T cell responses to HIV1 GAGp24-antigen specific pentameric peptides is expressed as the percentage of positive cells. Individual data points represent the HIV1-GAG CD8+ response in each animal. Horizontal lines denote the group geometric mean.

FIG. 6B. Magnitude and kinetics of CD8+ T cell responses in splenocytes following a priming dose and a boosting dose. CD8+ T cell responses to HIV1 GAGp24-antigen specific pentameric peptides is expressed as the percentage of positive cells. Individual data points represent the HIV1-GAG CD8+ response in each animal. Horizontal lines denote the group geometric mean.

FIG. 7A. CD8+ T cell responses to HIV-GAG prime boost regimens on days 30, 58 and 72 post prime. IFN-γ, TNF-α, IL-2 cytokine and CD107a responses are shown. Day 72 post-prime is day 14 post boost.

FIG. 7B. CD4+ T cell response to HIV-GAG prime boost regimes on days 30, 58 and 72 post prime. IFN-γ, TNF-α, IL-2 cytokine and CD107a responses are shown. Day 72 post-prime is day 14 post boost.

FIG. 8. Magnitude and kinetics of CD8+ responses in blood following a priming dose of a simian adenovirus encoding an HIV GAG transgene and a homologous or heterologous boosting dose. CD8+ T cell responses to HIV1 GAGp24-antigen specific pentameric peptides is expressed as the percentage of positive cells. Horizontal lines denote the group geometric mean.

FIG. 9A. CD8+ T cell responses to HIV-GAG prime boost regimens on days 28, 64, 72 and 100 post prime. IFN-γ, TNF-α, IL-2 cytokine and CD107a responses are shown. Day 72 post-prime is day 14 post-boost.

FIG. 9B. CD4+ T cell response to HIV-GAG prime boost regimes on days 28, 64, 72 and 100 post prime. IFN-γ, TNF-α, IL-2 cytokine and CD107a responses are shown. Day 72 post-prime is day 14 post-boost.

FIG. 10A. Polyfunctional CD8+ T cell response to immunization with ChAd-HSV Gly VI at doses of 5×10⁶ vp or 10⁸ vp. Responses of IFN-γ, TNF-α and/or IL-2 to the HSV Gly VI antigens ICP0, ICP4, UL-39, UL-47, UL-49 on day 20 are shown. Symbols represent T cell responses of individual mice. The median response is showed by solid horizontal lines.

FIG. 10B. Polyfunctional CD4+ T cell response to immunization with ChAd-HSV Gly VI at doses of 5×10⁶ vp or 10×10⁸ vp. Cytokine responses of IFN-γ, TNF-α and/or IL-2 to the HSV Gly VI antigens ICP0, ICP4, UL-39, UL-47, UL-49 on day 20 are shown. Symbols represent T cell responses of individual mice. The median response is showed by solid horizontal lines.

FIG. 11. Poly-functional CD8+ T cell profile of UL-47 response to immunization with adeno-HSV Gly VI at a dose of 10⁸ vp. Cytokine responses of IFN-γ, TNF-α and IL2 to the HSV Gly VI antigen on day 20 are shown compared to a saline control. Symbols represent the T cell responses of individual mice. The median response is showed by solid horizontal lines.

FIG. 12A. Polyfunctional CD8+ T cell response to immunization with adeno-HSV Gly VI at doses of 5×10⁶ vp or 10×10⁸ vp. The group immunized with 5×10⁶ vp was boosted with 1 μg SAM. Cytokine responses of IFN-γ, TNF-α and/or IL-2 to the HSV Gly VI antigens ICP0, ICP4, UL-39, UL-47, UL-49 on days 20 and 82 following the priming immunization (20P1) and day 25 following the boosting immunization. Circles represent T cell responses of individual mice. The median response is showed by solid horizontal lines.

FIG. 12B. Polyfunctional CD4+ T cell response to immunization with adeno-HSV Gly VI at doses of 5×10⁶ vp or 10×10⁸ vp. The group immunized with 5×10⁶ vp was boosted with 1 μg SAM. Cytokine responses of IFN-γ, TNF-α and/or IL-2 to the HSV Gly VI antigens ICP0, ICP4, UL-39, UL-47, UL-49 on days 20 and 82 following the priming immunization (20PI) and day 25 following the boosting immunization. Circles represent T cell responses of individual mice. The median response is showed by solid horizontal lines.

FIG. 13. Poly-functional CD8+ T cell profile of UL-47 response to a prime boost regimen with adeno-HSV Gly VI and SAM HSV Gly VI. Cytokine responses of IFN-γ, TNF-α and IL2 to the HSV Gly VI antigen on day 25 after heterologous prime/boost are shown compared to a saline control. Symbols represent T cell responses of individual mice. The median response is showed by solid horizontal lines.

### DETAILED DESCRIPTION OF THE INVENTION

Prime boost compositions and methods of the invention generate strong and lasting immune responses without inducing significant, or in some cases, without inducing detectable anti-vector immunity in the recipient. SAM vaccines are potent boosters of simian adenoviral vaccines and simian adenoviral vaccines are potent boosters of SAM vaccines. Heterologous prime/boost compositions and methods of the invention provide a potent and effective vaccine strategy, with the possibility of re-administering the same vaccine antigen multiple times without inducing anti-vector immunity.

The immune response can confer protective immunity, in which the vaccinated subject is able to control an infection with the pathological organism against which the vaccination was performed. The subject that develops a protective immune response may develop only mild to moderate symptoms of the disease caused by the pathological organism or no symptoms at all. The immune response can also be therapeutic, alleviating or eliminating the subject's response to the pathological organism against which the vaccination was performed.

Nucleic Acids

The term “nucleic acid” means a polymeric form of nucleotides of any length, which contain deoxyribonucleotides, ribonucleotides, and/or their analogs. It includes DNA, RNA and DNA/RNA hybrids. It also includes DNA or RNA analogs, such as those containing modified backbones (e.g. peptide nucleic acids (PNAs) or phosphorothioates) or modified bases. Thus, the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap.

The present inventors disclose herein nucleic acids comprising one or more nucleic acid sequence which encodes an antigen. A nucleic acid, as disclosed herein, can take various forms (e.g. single-stranded, double-stranded, vector, etc.). Nucleic acids may be circular or branched, but will typically be linear.

The nucleic acids used herein are preferably provided in purified or substantially purified form i.e., substantially free from other nucleic acids (e.g. free from naturally-occurring nucleic acids), particularly from host cell nucleic acids, typically being at least about 50% pure (by weight), and usually at least about 90% pure.

Nucleic acids may be prepared in many ways e.g., by chemical synthesis in whole or in part, by digesting longer nucleic acids using nucleases (e.g., restriction enzymes), by joining shorter nucleic acids or nucleotides (e.g., using ligases or polymerases) and from genomic or cDNA libraries.

The nucleic acids herein comprise a sequence which encodes at least one antigen. Typically, the nucleic acids of the invention will be in recombinant form, i.e., a form which does not occur in nature. For example, the nucleic acid may comprise one or more heterologous nucleic acid sequences (e.g., a sequence encoding another antigen and/or a control sequence such as a promoter or an internal ribosome entry site) in addition to the sequence encoding the antigen. The nucleic acid may be part of a vector, i.e., part of a nucleic acid construct designed for transduction/transfection of one or more cell types. Vectors may be, for example, expression vectors which are designed to express a nucleotide sequence in a host cell, or viral vectors which are designed to result in the production of a recombinant virus or virus-like particle.

Alternatively, or in addition, the sequence or chemical structure of the nucleic acid may be modified compared to a naturally-occurring sequence which encodes an antigen. The sequence of the nucleic acid molecule may be modified, e.g. to increase the efficacy of expression or replication of the nucleic acid, or to provide additional stability or resistance to degradation. Alternatively or additionally, a vaccine construct of the invention is resistant to RNAse digestion in an in vitro assay.

The nucleic acid encoding the polypeptides described above may be modified to increase translation efficacy and/or half-life. For example, the nucleic acid may be codon optimized or codon-pair optimized. A poly A tail (e.g., of about 30, about 40 or about 50 adenosine residues or more) may be attached to the 3′ end of the RNA to increase its half-life. The 5′ end of the RNA may be capped with a modified ribonucleotide with the structure m7G (5′)ppp(5′)N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). The cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O]N), which may further increase translation efficacy.

The nucleic acids may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g., cytosine (C), thymine (T), uracil (U), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or in or on the phosphate moiety. Many modified nucleosides and modified nucleotides are commercially available.

Nucleic acids of the invention may, for example, be an RNA-based vaccine. The RNA-based vaccine may comprise a self-amplifying RNA molecule. The self-amplifying RNA molecule may be an alphavirus-derived RNA replicon. Nucleic acids of the invention may be an adenovirus-based vaccine. The adenovirus-based vaccine may be a simian adenovirus.

Adenoviral Vectors

Adenoviruses are nonenveloped icosahedral viruses with a linear double stranded DNA genome of approximately 36 kb. Adenoviruses can transduce numerous cell types of several mammalian species, including both dividing and nondividing cells, without integrating into the genome of the host cell. They have been widely used for gene transfer applications due to their proven safety, ability to achieve highly efficient gene transfer in a variety of target tissues, and large transgene capacity. Human adenoviral vectors are currently used in gene therapy and vaccines but have the drawback of a high worldwide prevalence of pre-existing immunity following previous exposure to common human adenoviruses. Certain simian adenoviral vectors may demonstrate one or more of the following improved characteristics over other vectors: higher productivity, improved immunogenicity and increased transgene expression.

Adenoviruses have a characteristic morphology with an icosahedral capsid comprising three major proteins, hexon (II), penton base (III) and a knobbed fiber (IV), along with a number of other minor proteins, VI, VIII, IX, IIIa and IVa2. The hexon accounts for the majority of the structural components of the capsid, which consists of 240 trimeric hexon capsomeres and 12 penton bases. The hexon has three conserved double barrels and the top has three towers, each tower containing a loop from each subunit that forms most of the capsid. The base of the hexon is highly conserved between adenoviral serotypes, while the surface loops are variable. The penton is another adenoviral capsid protein; it forms a pentameric base to which the fiber attaches. The trimeric fiber protein protrudes from the penton base at each of the 12 vertices of the capsid and is a knobbed rod-like structure. The primary role of the fiber protein is to tether the viral capsid to the cell surface via the interaction of the knob region with a cellular receptor. Variations in the flexible shaft, as well as knob regions of fiber, are characteristic of the different adenoviral serotypes. The adenoviral fiber protein plays an important role in receptor binding and immunogenicity of adenoviral vectors.

The adenoviral genome has been well characterized. The linear, double-stranded DNA is associated with the highly basic protein VII and a small peptide pX (also termed mu). Another protein, V, is packaged with this DNA-protein complex and provides a structural link to the capsid via protein VI. There is general conservation in the overall organization of the adenoviral genome with respect to specific open reading frames being similarly positioned, e.g. the location of the E1A, E1B, E2A, E2B, E3, E4, L1, L2, L3, L4 and L5 genes of each virus. Each extremity of the adenoviral genome comprises a sequence known as an inverted terminal repeat (ITR), which is necessary for viral replication. The 5′ end of the adenoviral genome contains the 5′ cis-elements necessary for packaging and replication; i.e., the 5′ ITR sequences (which can function as origins of replication) and the native 5′ packaging enhancer domains, which contain sequences necessary for packaging linear adenoviral genomes and enhancer elements for the E1 promoter. The 3′ end of the adenoviral genome includes 3′ cis-elements, including the ITRs, necessary for packaging and encapsidation. The virus also comprises a virus-encoded protease, which is necessary for processing some of the structural proteins required to produce infectious virions.

The structure of the adenoviral genome is described on the basis of the order in which the viral genes are expressed following host cell transduction. More specifically, the viral genes are referred to as early (E) or late (L) genes according to whether transcription occurs prior to or after onset of DNA replication. In the early phase of transduction, the E1A, E1B, E2A, E2B, E3 and E4 genes of adenovirus are expressed to prepare the host cell for viral replication. The E1 gene is considered a master switch, it acts as a transcription activator and is involved in both early and late gene transcription. E2 is involved in DNA replication; E3 is involved in immune modulation and E4 regulates viral mRNA metabolism. During the late phase of infection, expression of the late genes L1-L5, which encode the structural components of the viral particles, is activated. Late genes are transcribed from the Major Late Promoter (MLP) with alternative splicing.

Historically, adenovirus vaccine development has focused on defective, non-replicating vectors. They are rendered replication defective by deletion of the E1 region genes, which are essential for replication. Typically, non-essential E3 region genes are also deleted to make room for exogenous transgenes. An expression cassette comprising the transgene under the control of an exogenous promoter is then inserted. These replication-defective viruses can then be produced in E1-complementing cells. Replication competent adenoviral vectors can also be vehicles for delivering vaccine antigens. Human replication competent adenoviruses have been safely administered to adult humans in clinical trials directed to infectious diseases and oncological indications.

The term “replication-defective” or “replication-incompetent” adenovirus refers to an adenovirus that is incapable of replication because it has been engineered to comprise at least a functional deletion (or “loss-of-function” mutation), i.e. a deletion or mutation which impairs the function of a gene without removing it entirely, e.g. introduction of artificial stop codons, deletion or mutation of active sites or interaction domains, mutation or deletion of a regulatory sequence of a gene etc., or a complete removal of a gene encoding a gene product that is essential for viral replication, such as one or more of the adenoviral genes selected from E1A, E1B, E2A, E2B, E3 and E4 (such as E3 ORF1, E3 ORF2, E3 ORF3, E3 ORF4, E3 ORF5, E3 ORF6, E3 ORF7, E3 ORF8, E3 ORF9, E4 ORF7, E4 ORF6, E4 ORF4, E4 ORF3, E4 ORF2 and/or E4 ORF1). Suitably, E1 and optionally E3 and/or E4 are deleted. If deleted, the aforementioned deleted gene region will suitably not be considered in the alignment when determining percent identity with respect to another sequence.

The term “replication-competent” adenovirus refers to an adenovirus which can replicate in a host cell in the absence of any recombinant helper proteins comprised in the cell. Suitably, a replication-competent adenovirus comprises intact structural genes and the following intact or functionally essential early genes: E1A, E1B, E2A, E2B and E4. Wild type adenoviruses isolated from a particular animal will be replication competent in that animal.

The choice of gene expression cassette insertion sites of replication defective vectors has been primarily focused on replacing regions known to be involved in viral replication. The choice of gene expression cassette insertion sites of replication competent vectors must preserve the replication machinery. Viruses maximize their coding capacity by generating highly complex transcription units controlled by multiple promoters and alternative splicing. Consequently, replication competent viral vectors must preserve the sequences necessary for replication while allowing room for functional expression cassettes.

In embodiments of the invention, the E1 region or fragments thereof necessary for replication are present and the exogenous sequence of interest is inserted into the fully or partially deleted E3 region. In an embodiment, the vector comprises a left ITR region, followed by an E1 region, then the E3 region, which is substituted with an expression cassette comprising a promoter, an antigen of interest and, optionally, additional enhancer elements; these are followed by a fiber region, an E4 region and a right ITR; translation occurs in a rightward direction.

The term adenoviral “vector” refers to at least one adenoviral polynucleotide or to a mixture of at least one polynucleotide and at least one polypeptide capable of introducing a polynucleotide into a cell. “Low seroprevalence” may mean having a reduced pre-existing neutralizing antibody level as compared to human adenovirus 5 (Ad5). Similarly or alternatively, “low seroprevalence” may mean less than about 40% seroprevalence, less than about 30% seroprevalence, less than about 20% seroprevalence, less than about 15% seroprevalence, less than about 10% seroprevalence, less than about 5% seroprevalence, less than about 4% seroprevalence, less than about 3% seroprevalence, less than about 2% seroprevalence, less than about 1% seroprevalence or no detectable seroprevalence. Seroprevalence can be measured as the percentage of individuals having a clinically relevant neutralizing titer (defined as a 50% neutralisation titer >200) using methods as described by Aste-Amezaga et al. (2004) Hum. Gene Ther. 15:293.

In an embodiment, an adenoviral vector of the present invention is derived from a nonhuman simian adenovirus, also referred to as a “simian adenovirus.” Numerous adenoviruses have been isolated from nonhuman simians such as chimpanzees, bonobos, rhesus macaques, orangutans and gorillas. Vectors derived from these adenoviruses can induce strong immune responses to transgenes encoded by these vectors. Certain advantages of vectors based on nonhuman simian adenoviruses include a relative lack of cross-neutralizing antibodies to these adenoviruses in the human target population, thus their use overcomes the pre-existing immunity to human adenoviruses. For example, some simian adenoviruses have no cross reactivity with preexisting human neutralizing antibodies and cross-reaction of certain chimpanzee adenoviruses with pre-existing human neutralizing antibodies is only present in 2% of the target population, compared with 35% in the case of certain candidate human adenovirus vectors (Colloca et al. (2012) Sci. Transl. Med. 4:1).

Adenoviral vectors of the invention may be derived from a non-human adenovirus, such as a simian adenovirus, e.g., from chimpanzees (Pan troglodytes), bonobos (Pan paniscus), gorillas (Gorilla gorilla), rhesus macaques (Macaca mulatta) and orangutans (Pongo abelii and Pongo pygnaeus). They include adenoviruses from Group B, Group C, Group D, Group E and Group G. Chimpanzee adenoviruses include, but are not limited to AdY25, ChAd3, ChAd15, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, SadV41 and ChAd157. Alternatively, adenoviral vectors may be derived from nonhuman simian adenoviruses isolated from bonobos, such as PanAd1, PanAd2, PanAd3, Pan 5, Pan 6, Pan 7 (also referred to as C7) and Pan 9. Vectors may include, in whole or in part, a nucleotide encoding the fiber, penton or hexon of a non-human adenovirus.

In an embodiment of the adenoviral vectors of the invention, the adenovirus has a seroprevalence of less than about 40% seroprevalence, preferably less than about 30% seroprevalence, less than about 20% seroprevalence, less than about 15% seroprevalence, less than about 10% seroprevalence, less than about 5% seroprevalence, less than about 4% seroprevalence, less than about 3% seroprevalence, less than about 2% seroprevalence, less than about 1%, more preferably no seroprevalence in human subjects and most preferably no seroprevalence in human subjects that have not previously been in contact with a simian adenovirus.

In embodiments of the adenoviral vectors of the invention, the adenoviral DNA is capable of entering a mammalian target cell, i.e. it is infectious. An infectious recombinant adenovirus of the invention can be used as a prophylactic or therapeutic vaccine and for gene therapy. Thus, in an embodiment, the recombinant adenovirus comprises an endogenous molecule for delivery into a target cell. The target cell is in the class Mammalia. Target cells may be derived from mammals in the subclasses Prototheria, Metatheria and Eutheria, including but not limited to those in the orders artiodactyla, carnivore, lagomorpha, primates and rodentia. By way of example, the cell may be a bovine cell, a canine cell, a caprine cell, a cervine cell, a chimpanzee cell, a chiroptera cell, an equine cell, a feline cell, a human cell, a lupine cell, an ovine cell, a porcine cell, a rodent cell, an ursine cell or a vulpine cell. In a preferred embodiment, the cell is a human cell. The endogenous molecule for delivery into a target cell can be an expression cassette.

In an embodiment of the invention, the vector is a functional or an immunogenic derivative of an adenoviral vector. By “derivative of an adenoviral vector” is meant a modified version of the vector, e.g., one or more nucleotides of the vector are deleted, inserted, modified or substituted.

Self-Amplifying RNA

The term “RNA vaccine” encompasses all vaccines comprising the nucleic acid RNA and encode one or more nucleotide sequence encoding an antigen capable of inducing an immune response in a mammal.

“Self-amplifying RNA,” “self-replicating RNA” and “RNA replicon” are used interchangeably to mean RNA with the ability to replicate itself. The term “self-amplifying RNA vector” refers to a self-amplifying RNA capable of introducing a polynucleotide into a cell. The self-amplifying RNA vectors of the invention comprise mRNA encoding one or more antigens. These mRNAs can replace nucleic acid sequences encoding structural proteins required for the production of infectious virus. The RNA can be produced in vitro by enzymatic transcription, thereby avoiding manufacturing issues associated with cell culture production of vaccines. After immunization with a self-amplifying RNA molecule of the invention, replication and amplification of the RNA molecule occur in the cytoplasm of the transfected cell and the nucleic acid is not integrated into the genome. As the RNA does not integrate into the genome and transform the target cell, self-amplifying RNA vaccines do not pose the safety hurdles faced by some recombinant DNA vaccines.

Self-amplifying RNA molecules are known in the art and can be produced by using replication elements derived from, e.g., alphaviruses, and substituting structural viral proteins with a nucleotide sequence encoding a protein of interest. A self-amplifying RNA molecule is typically a plus-strand molecule which can be directly translated after delivery to a cell. This translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen or may be transcribed to provide further transcripts with the same sense as the delivered RNA, which are then translated to provide in situ expression of the antigen. The overall result of this sequence of transcriptions is a huge amplification in the number of the introduced replicon RNAs; the encoded antigen becomes a major polypeptide product of the cells.

One suitable system for achieving self-replication in this manner is to use an alphavirus-based replicon. These replicons are plus-stranded RNAs which lead to the translation of a replicase (or replicase-transcriptase) following their delivery to a cell. The replicase is translated as a polyprotein which auto-cleaves to provide a replication complex which creates genomic-strand copies of the plus-strand delivered RNA. These minus-strand transcripts can themselves be transcribed to give further copies of the plus-stranded parent RNA and also to give a subgenomic transcript which encodes the antigen. Translation of the subgenomic transcript leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons.

As used herein, the term “alphavirus” has its conventional meaning in the art and includes various species such as Venezuelan equine encephalitis virus (VEE e.g., Trinidad donkey, TC83CR, etc.), Semliki Forest virus (SFV), Sindbis virus, Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O′ nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. The term alphavirus may also include chimeric alphaviruses that contain genome sequences from more than one alphavirus.

An “alphavirus replicon particle” or “replicon particle,” i.e. a VRP, is an alphavirus replicon packaged with alphavirus structural proteins. In an embodiment, a replicon particle is distinct from a VRP.

An “alphavirus replicon” (or “replicon”) is an RNA molecule which can direct its own amplification in vivo in a target cell. The replicon encodes the polymerase(s) which catalyzes RNA amplification and contains cis RNA sequences required for replication which are recognized and utilized by the encoded polymerase(s). An alphavirus replicon typically contains the following ordered elements: 5′ viral sequences required in cis for replication, sequences which encode biologically active alphavirus nonstructural proteins (nsP1, nsP2, nsP3, nsP4), 3′ viral sequences required in cis for replication, and a polyadenylate tract. An alphavirus replicon also may contain one or more viral subgenomic junction region promoters directing the expression of heterologous nucleotide sequences, which may be modified in order to increase or reduce viral transcription of the subgenomic fragment and heterologous sequence(s) to be expressed.

Self-amplifying RNAs contain the basic elements of mRNA, i.e., a cap, 5′UTR, 3′UTR and a poly(A) tail. They additionally comprise a large open reading frame (ORF) that encodes non-structural viral genes and one or more subgenomic promoter. The nonstructural genes, which include a polymerase, form intracellular RNA replication factories and transcribe the subgenomic RNA at high levels. This mRNA encoding the vaccine antigen(s) is amplified in the cell, resulting in high levels of mRNA and antigen expression.

Alternatively or additionally, the self-amplifying RNA molecules described herein encode (i) an RNA-dependent RNA polymerase which can transcribe RNA from the self-amplifying RNA molecule and (ii) an antigen. The polymerase can be an alphavirus replicase e.g., comprising one or more of the non-structural alphavirus proteins nsP1, nsP2, nsP3 and nsP4.

Whereas natural alphavirus genomes encode structural virion proteins in addition to the non-structural replicase polyprotein, alternatively or additionally, the self-amplifying RNA molecules do not encode alphavirus structural proteins. Thus, the self-amplifying RNA can lead to the production of genomic RNA copies of itself in a cell, but not to the production of RNA-containing virions. The inability to produce these virions means that, unlike a wild-type alphavirus, the self-amplifying RNA molecule cannot perpetuate itself in infectious form. The alphavirus structural proteins which are necessary for perpetuation in wild-type viruses are absent from self-amplifying RNAs of the present disclosure and their place is taken by a gene(s) encoding the immunogen of interest, such that the subgenomic transcript encodes the immunogen rather than the structural alphavirus virion proteins.

A self-amplifying RNA molecule useful with the invention may have at least two open reading frames. The first open reading frame encodes a replicase; the second open reading frame encodes an antigen. Alternatively or additionally, the RNA may have one or more additional (e.g. downstream) open reading frames, e.g. to encode further antigen(s) or to encode accessory polypeptides.

Alternatively or additionally, the self-amplifying RNA molecule disclosed herein has a 5′ cap (e.g. a 7-methylguanosine). This cap can enhance in vivo translation of the RNA. Alternatively or additionally, the 5′ sequence of the self-amplifying RNA molecule must be selected to ensure compatibility with the encoded replicase.

A self-amplifying RNA molecule can have a 3′ poly-A tail. It may also include a poly-A polymerase recognition sequence (e.g. AAUAAA) near its 3′ end.

Self-amplifying RNA molecules can have various lengths, but they are typically 5000-25000 nucleotides long. Self-amplifying RNA molecules will typically be single-stranded. Single-stranded RNAs can generally initiate an adjuvant effect by binding to TLR7, TLR8, RNA helicases and/or dsRNA-dependent protein kinase (PKR). RNA delivered in double-stranded form (dsRNA) can bind to TLR3, and this receptor can also be triggered by dsRNA which is formed either during replication of a single-stranded RNA or within the secondary structure of a single-stranded RNA.

The self-amplifying RNA can conveniently be prepared by in vitro transcription (IVT). IVT can use a cDNA template created and propagated in plasmid form in bacteria, or created synthetically, for example by gene synthesis and/or polymerase chain-reaction (PCR) engineering methods. For example, a DNA-dependent RNA polymerase, such as the bacteriophage T7, T3 or SP6 RNA polymerases, can be used to transcribe the self-amplifying RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

The self-amplifying RNA can include, alternatively or in addition to any 5′ cap structure, one or more nucleotides having a modified nucleobase. An RNA used with the invention preferably includes only phosphodiester linkages between nucleosides, but in some embodiments, it can contain phosphoramidate, phosphorothioate, and/or methylphosphonate linkages.

The self-amplifying RNA molecule may encode a single heterologous polypeptide antigen or, optionally, two or more heterologous polypeptide antigens linked together in a way that each of the sequences retains its identity (e.g., linked in series) when expressed as an amino acid sequence. The heterologous polypeptides generated from the self-amplifying RNA may then be produced as a fusion polypeptide or engineered in such a manner as to result in separate polypeptide or peptide sequences.

The self-amplifying RNA molecules described herein may be engineered to express multiple nucleotide sequences, from two or more open reading frames, thereby allowing co-expression of proteins, such as one, two or more antigens.

A synthetic SAM vaccine is herein produced through rapid, generic and cell-free processes, with the potential to produce millions of doses in a short timeframe. It is provided along with adenoviral based vaccines to produce potent humoral and cellular immunity.

Lipid-Based Delivery Systems for Self-Amplifying RNA

The RNA vaccines of the invention may comprise a lipid-based delivery system. These systems can efficiently deliver an RNA molecule to the interior of a cell, where it can then replicate and express the encoded antigen(s).

The delivery system may have adjuvant effects which enhance the immunogenicity of the encoded antigen. For example, the nucleic acid molecule may be encapsulated in liposomes or non-toxic biodegradable polymeric microparticles. “Liposomes” are uni- or multilamellar lipid structures enclosing an aqueous interior.

In an embodiment, the nucleic acid-based vaccine comprises a lipid nanoparticle (LNP) delivery system. Alternatively or additionally, the nucleic molecule may be delivered as a cationic nanoemulsion (CNE). Alternatively or additionally, the nucleic acid-based vaccine may comprise a naked nucleic acid, such as naked RNA (e.g. mRNA), but lipid-based delivery systems are preferred.

“Lipid nanoparticles (LNPs)” are non-virion liposome particles in which a nucleic acid molecule (e.g. RNA) can be encapsulated. LNP delivery systems and non-toxic biodegradable polymeric microparticles, and methods for their preparation are known in the art. The particles can include some external RNA (e.g. on the surface of the particles), but at least half of the RNA (and preferably all of it) is encapsulated. Liposomal particles can, for example, be formed of a mixture of zwitterionic, cationic and anionic lipids which can be saturated or unsaturated, for example 1,2-distearoyl-sn-glycero-3-phosphocholine (DSPC) (zwitterionic, saturated), 1,2-dilinoleyoxy-3-dimethylaminopropane (DlinDMA) (cationic, unsaturated), and/or 1,2-dimyristoyl-rac-glycerol (DMG) (anionic, saturated). The liposomes will typically comprise helper lipids. Useful helper lipids include zwitterionic lipids, such as DPPC, DOPC, DSPC, dodecylphosphocholine, 1,2-dioleoyl-sn-glycero-3-phosphatidylethanolamine (DOPE), and 1,2-diphytanoyl-sn-glycero-3-phosphoethanolamine (DPyPE); sterols, such as cholesterol; and PEGylated lipids, such as PEG-DMPE (PEG-conjugated 1, 2-dimyristoyl-Sn-glycero-3-phosphoethanolamine-N-[methoxy (polyethylene glycol)]) or PEG-DMG (PEG-conjugated 1,2-Dimyristoyl-sn-glycerol, methoxypolyethylene Glycol). In some embodiments, useful PEGylated lipids may be PEG2K-DMPE (PEG-conjugated 1, 2-dimyristoyl-Sn-glycero-3-phosphoethanolamine-N-[methoxy (polyethylene glycol)-2000]) or PEG2K-DMG (PEG-conjugated 1,2-Dimyristoyl-sn-glycerol, methoxypolyethylene Glycol-2000). Preferred LNPs for use with the invention include a zwitterionic lipid which can form liposomes, optionally in combination with at least one cationic lipid (such as N-[1-(2,3-Dioleoyloxy)propyl]-N,N,N-trimethylammonium methyl-sulfate (DOTAPBis(2-methacryloyl)oxyethyl disulfide (DSDMA), 2,3-Dioleyloxy-1-(dimethylamino)propane (DODMA), 1,2-dilinoleyoxy-3-dimethylaminopropane (DLinDMA), N,N-dimethyl-3-aminopropane (DLenDMA), etc.). A mixture of DSPC, DlinDMA, PEG-DMG and cholesterol is particularly effective. Alternatively or additionally, the LNPs are liposomes comprising RV01.

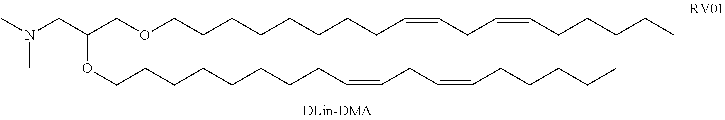 [SMILES: CCCCC/C=C\C/C=C\CCCCCCCCOCC(CN(C)C)OCCCCCCCC/C=C\C/C=C\CCCCC]

Alternatively or additionally, the LNP comprises neutral lipids, cationic lipids, cholesterol and polyethylene glycol (PEG) and forms nanoparticles that encompass the self-amplifying RNA. In some embodiments, the cationic lipids herein comprise the structure of Formula I:

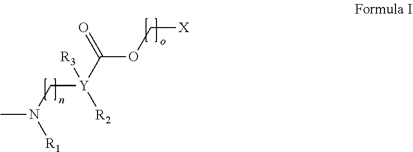

wherein n=an integer from 1 to 3 and
(i) R₁ is CH₃, R₂ and R₃ are both H, and Y is C; or
(ii) R₁ and R₂ are collectively CH₂—CH₂ and together with the nitrogen form a five-, six-, or seven-membered heterocycloalkyl, R₃ is CH₃, and Y is C; or
(iii) R₁ is CH₃, R₂ and R₃ are both absent, and Y is O;
wherein o is 0 or 1;
wherein X is:
(i)

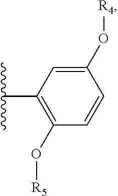

wherein R₄ and R₅ are independently a C₁₀₋₂₀ hydrocarbon chain having one or two cis alkene groups at either or both of the omega 6 and 9 positions; or
(ii) —CH(—R₆)—R₇, wherein
(1) R₆ is —(CH₂)ₚ—O—C(O)—R₈ or —Cₚ—R₈;
(2) R₇ is —(CH₂)ₚ—O—C(O)—R_{8′} or —Cₚ—R_{8′},
(3) p and p′ are independently 0, 1, 2, 3 or 4; and
(4) R₈ and R_{8′} are independently a
   (A) —C₈₋₂₀ hydrocarbon chain having one or two cis alkene groups at either or both of the omega 6 and 9 positions;
   (B) —C₁₋₃—C(—O—C₆₋₁₂)—O—C₆₋₁₂ saturated or unsaturated hydrocarbon chain;
   (C) —C₆₋₁₆ saturated hydrocarbon chain;
   (D) —C(—C₆₋₁₆)—C₆₋₁₆ saturated or unsaturated hydrocarbon chain;
   (E) —C[—C—O—C(O)—C₄₋₁₂]—C—O—C(O)—C₄₋₁₂ saturated or unsaturated hydrocarbon chain; and
   (F) —C₆₋₁₆ saturated or unsaturated hydrocarbon chain.

In an embodiment, R₁ is CH₃, R₂ and R₃ are both H, and Y is C. In some embodiments, R₁ and R₂ are collectively CH₂CH₂ and together with the nitrogen form a five-, six-, or seven-membered heterocycloalkyl, R₃ is CH₃, and Y is C. In some embodiments, R₁ is CH₃, R₂ and R3 are both absent, and Y is O.

In an embodiment, X is

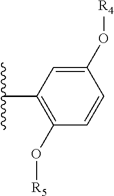

wherein R₄ and R₅ are independently a C₁₀₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇—C_{p′}—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —(CH₂)ₚ—O—C(O)—R₈, R₇ is C_{p′}—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —(CH₂)ₚ—O—C(O)—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —Cₚ—R₈, R₇ is —(CH₂)_{p′}—O—C(O)—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —(CH₂)ₚ—O—C(O)—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —(CH₂)_{p′}—O—C(O)—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₈₋₂₀ hydrocarbon chain having one or two cis alkene groups at either or both of the omega 6 and 9 positions; and R₈′ is a —C₆₋₁₆ saturated or unsaturated hydrocarbon chain.

In an embodiment, X is —CH(—R₆)—R₇, R₆ is —C_{p′}—R₈, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —C_{p′}—R₈′, p and p′ are independently 0, 1, 2, 3 or 4; R₈ is —C₁₋₃—C(—O—C₆₋₁₂)—O—C₆₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇—C_{p′}—R₈′, p and p′ are independently 0, 1, 2, 3 or 4; R₈ is a —C₁₋₃—C(—O—C₆₋₁₂)—O—C₆₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₆₋₁₆ saturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₆₋₁₆ saturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₆₋₁₆ saturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₆₋₁₆ saturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; R₈ is a —C₆₋₁₆ saturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C(—C₆₋₁₆)—C₆₋₁₆ saturated or unsaturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇—C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇—C_{p′}—R₈′, p and p′ are independently 0, 1, 2, 3 or 4; and R₈ is a —C[—C—O—C(O)—C₄₋₁₂]—C—O—C(O)—C₄₋₁₂ saturated or unsaturated hydrocarbon chain; and R₈′ is a —C₆₋₁₆ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₈₋₂₀ hydrocarbon chain having one or two cis alkene groups at either or both of the omega 6 and 9 positions.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₁₋₃—C(—O—C₆₋₁₂)—O—C₆₋₁₂ saturated or unsaturated hydrocarbon chain.

In an embodiment, X is —CH(—R₆)—R₇, R₆ is —C_{p′}—R₈, R₇—C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C(—C₆₋₁₆)—C₆₋₁₆ saturated or unsaturated hydrocarbon chain.

In an embodiment, X is —CH(—R₆)—R₇, R₆ is —Cₚ—R₈, R₇ is —C_{p′}—R₈″, p and p′ are independently 0, 1, 2, 3 or 4; and R₈ is a —C₆₋₁₆ saturated or unsaturated hydrocarbon chain; and R₈′ is a —C[—C—O—C(O)—C₄₋₁₂]—C—O—C(O)—C₄₋₁₂ saturated or unsaturated hydrocarbon chain.

In an embodiment, X is —CH(—R₆)—R₇, R₆ is —C_{p′}—R₈, R₇ is —C_{p′}—R₈′, p and p′ are independently 0, 1, 2, 3 or 4; and R₈ is a —C₆₋₁₆ saturated or unsaturated hydrocarbon chain; and R₈′ is a —C₆₋₁₆ saturated or unsaturated hydrocarbon chain.

In an embodiment, an exemplary cationic lipid is RV28 having the following structure:

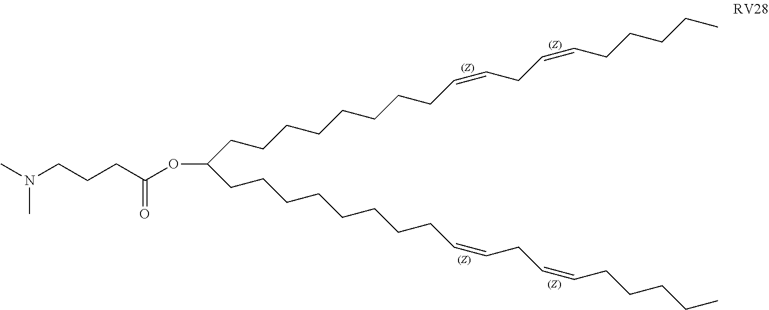 [SMILES: CCCCC/C=C\C/C=C\CCCCCCCCC(CCCCCCCC/C=C\C/C=C\CCCCC)OC(=O)CCCN(C)C]

In an embodiment, an exemplary cationic lipid is RV31 having the following structure:

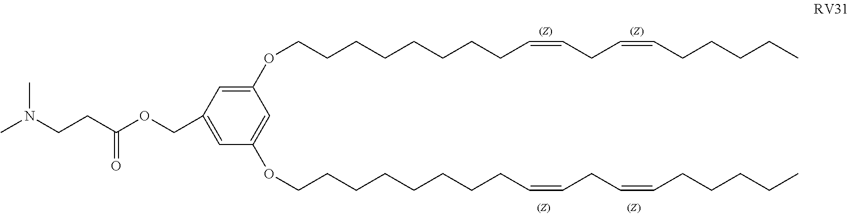 [SMILES: CCCCC/C=C\C/C=C\CCCCCCCCOc1cc(COC(=O)CCN(C)C)cc(OCCCCCCCC/C=C\C/C=C\CCCCC)c1]

In an embodiment, an exemplary cationic lipid is RV33 having the following structure:

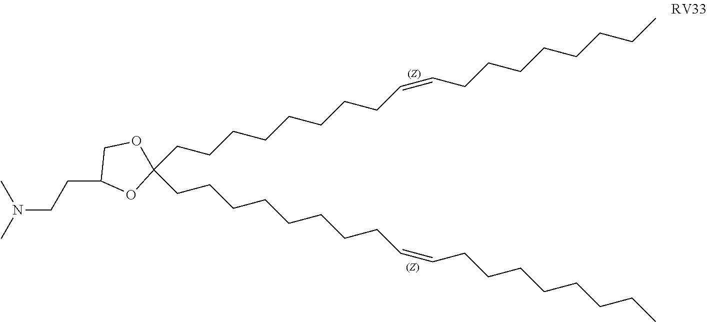 [SMILES: CCCCCCCC/C=C\CCCCCCCCC1(CCCCCCCC/C=C\CCCCCCCC)OCC(CCN(C)C)O1]

In an embodiment, an exemplary cationic lipid is RV37 having the following structure:

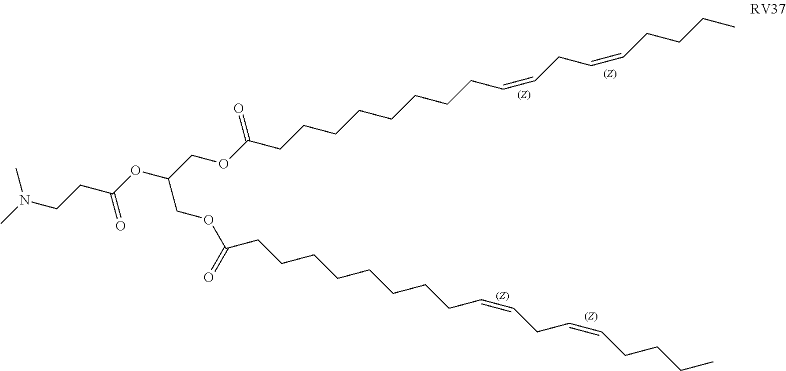 [SMILES: CCCC/C=C\C/C=C\CCCCCCCCC(=O)OCC(COC(=O)CCCCCCCC/C=C\C/C=C\CCCC)OC(=O)CCN(C)C]

In an embodiment, the LNP comprises the cationic lipid RV39, i.e., 2,5-bis((9Z,12Z)-octadeca-9,12-dien-1-yloxy)benzyl 4-(dimethylamino)butanoate):

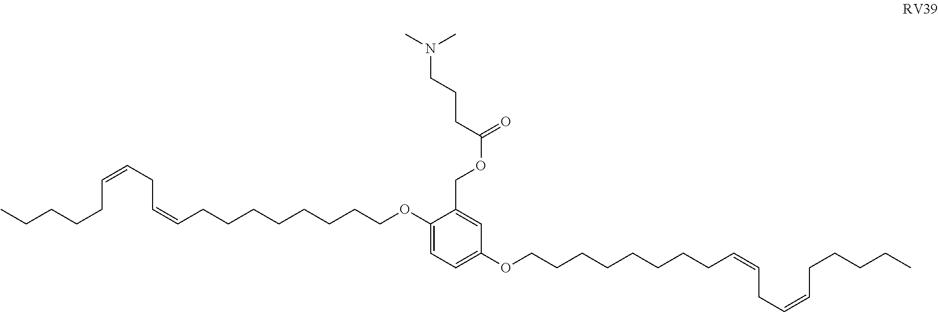 [SMILES: CCCCC/C=C\C/C=C\CCCCCCCCOc1ccc(OCCCCCCCC/C=C\C/C=C\CCCCC)c(COC(=O)CCCN(C)C)c1]

In an embodiment, an exemplary cationic lipid is RV42 having the following structure:

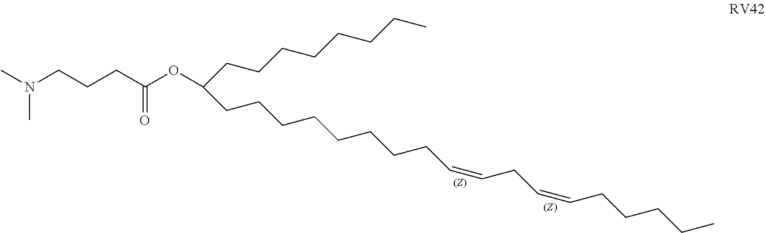 [SMILES: CCCCC/C=C\C/C=C\CCCCCCCCC(CCCCCCCC)OC(=O)CCCN(C)C]

In an embodiment, an exemplary cationic lipid is RV44 having the following structure:

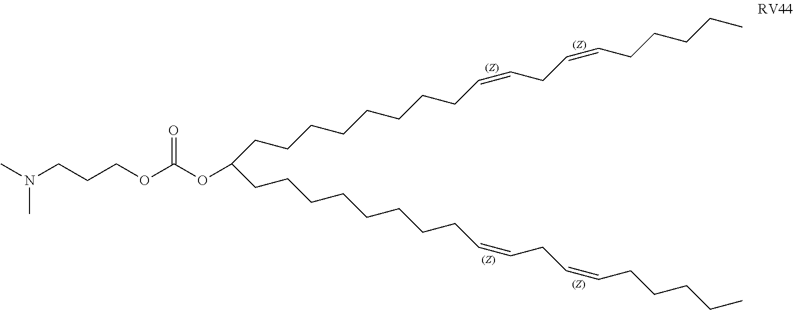 [SMILES: CCCCC/C=C\C/C=C\CCCCCCCCC(CCCCCCCC/C=C\C/C=C\CCCCC)OC(=O)OCCCN(C)C]

In an embodiment, an exemplary cationic lipid is RV73 having the following structure:

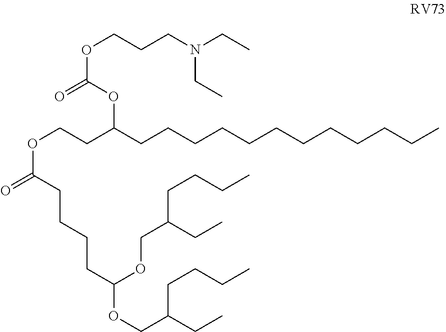 [SMILES: CCCCCCCCCCCCC(CCOC(=O)CCCCC(OCC(CC)CCCC)OCC(CC)CCCC)OC(=O)OCCCN(CC)CC]

In an embodiment, an exemplary cationic lipid is RV75 having the following structure:

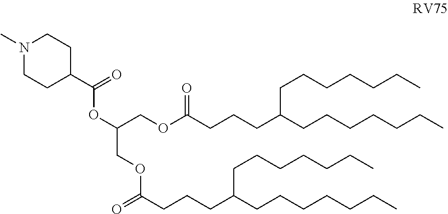 [SMILES: CCCCCCCC(CCCCCCC)CCCC(=O)OCC(COC(=O)CCCC(CCCCCCC)CCCCCCC)OC(=O)C1CCN(C)CC1]

In an embodiment, an exemplary cationic lipid is RV81 having the following structure:

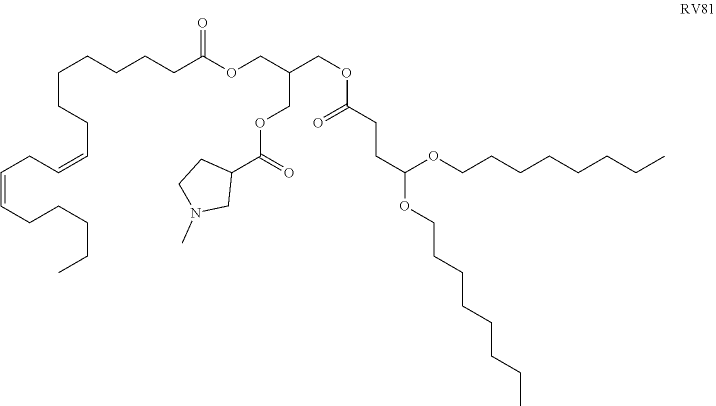 [SMILES: CCCCC/C=C\C/C=C\CCCCCCCC(=O)OCC(COC(=O)CCC(OCCCCCCCC)OCCCCCCCC)COC(=O)C1CCN(C)C1]

In an embodiment, an exemplary cationic lipid is RV84 having the following structure:

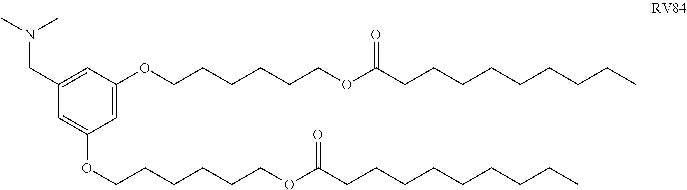 [SMILES: CCCCCCCCCC(=O)OCCCCCCOc1cc(CN(C)C)cc(OCCCCCCOC(=O)CCCCCCCCC)c1]

In an embodiment, an exemplary cationic lipid is RV85 having the following structure:

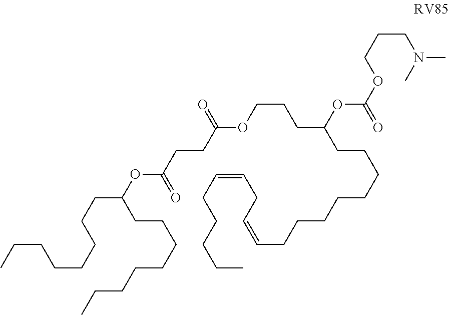 [SMILES: CCCCC/C=C\C/C=C\CCCCCCCCC(CCCOC(=O)CCC(=O)OC(CCCCCCCC)CCCCCCCC)OC(=O)OCCCN(C)C]

In an embodiment, an exemplary cationic lipid is RV86 having the following structure:

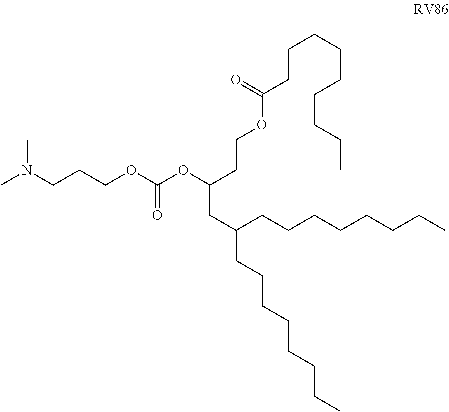 [SMILES: CCCCCCCCCC(=O)OCCC(CC(CCCCCCCC)CCCCCCCC)OC(=O)OCCCN(C)C]

In an embodiment, an exemplary cationic lipid is RV88 having the following structure:

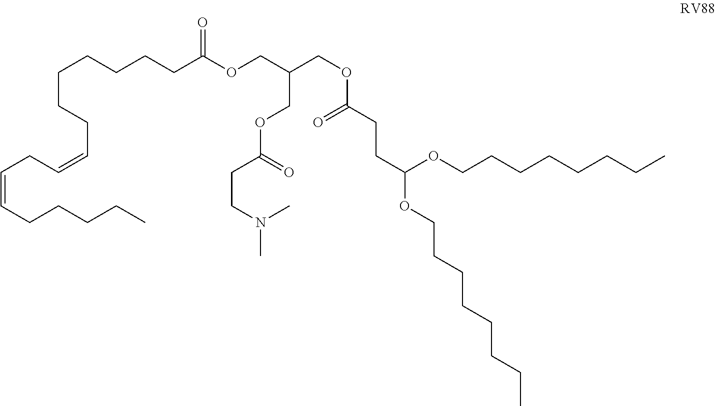 [SMILES: CCCCC/C=C\C/C=C\CCCCCCCC(=O)OCC(COC(=O)CCC(OCCCCCCCC)OCCCCCCCC)COC(=O)CCN(C)C]

In an embodiment, an exemplary cationic lipid is RV91 having the following structure:

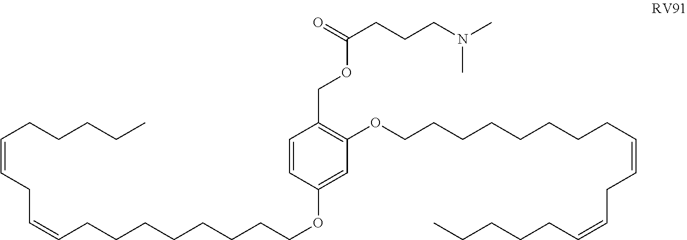 [SMILES: CCCCC/C=C\C/C=C\CCCCCCCCOc1ccc(COC(=O)CCCN(C)C)c(OCCCCCCCC/C=C\C/C=C\CCCCC)c1]

In an embodiment, an exemplary cationic lipid is RV92 having the following structure:

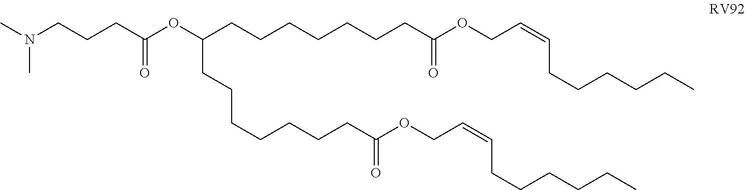 [SMILES: CCCCCC/C=C\COC(=O)CCCCCCCC(CCCCCCCC(=O)OC/C=C\CCCCCC)OC(=O)CCCN(C)C]

In an embodiment, an exemplary cationic lipid is RV93 having the following structure:

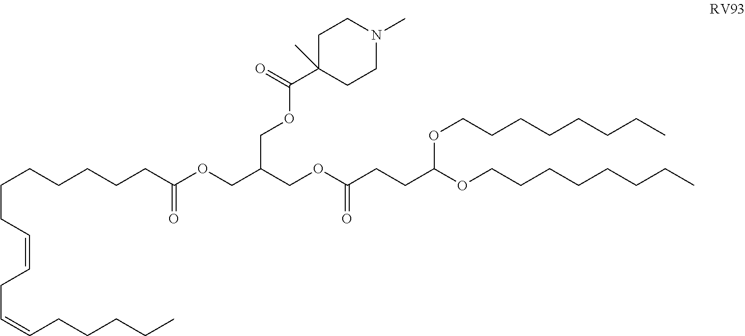 [SMILES: CCCCC/C=C\C/C=C\CCCCCCCC(=O)OCC(COC(=O)CCC(OCCCCCCCC)OCCCCCCCC)COC(=O)C1(C)CCN(C)CC1]

In an embodiment, an exemplary cationic lipid is 2-(5-((4-((1,4-dimethylpiperidine-4-carbonyl)oxy)hexadecyl)oxy)-5-oxopentyl)propane-1,3-diyl dioctanoate (RV94), having the following structure:

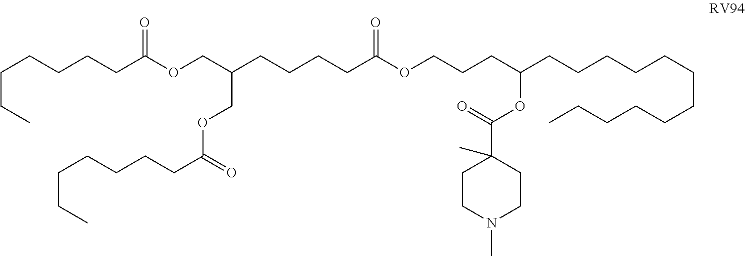 [SMILES: CCCCCCCCCCCCC(CCCOC(=O)CCCCC(COC(=O)CCCCCCC)COC(=O)CCCCCCC)OC(=O)C1(C)CCN(C)CC1]

In an embodiment, an exemplary cationic lipid is RV95 having the following structure:

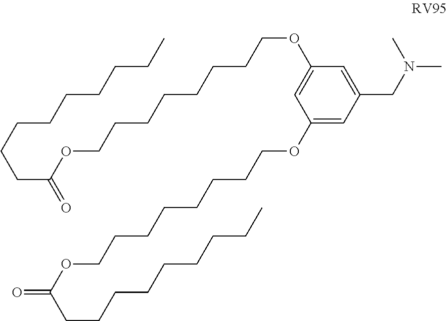 [SMILES: CCCCCCCCCC(=O)OCCCCCCCCOc1cc(CN(C)C)cc(OCCCCCCCCOC(=O)CCCCCCCCC)c1]

In an embodiment, an exemplary cationic lipid is RV96 having the following structure:

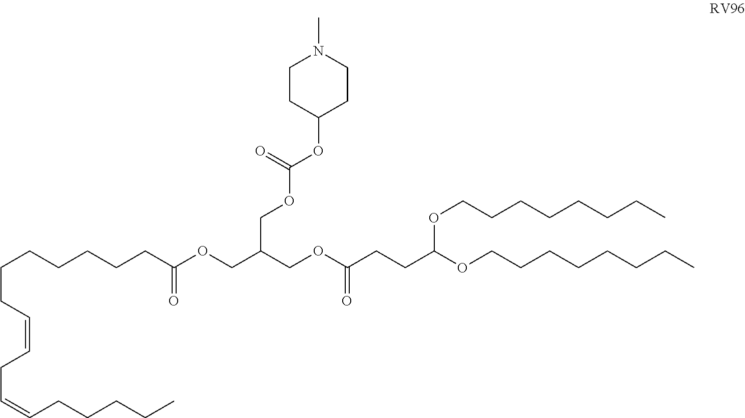 [SMILES: CCCCC/C=C\C/C=C\CCCCCCCC(=O)OCC(COC(=O)CCC(OCCCCCCCC)OCCCCCCCC)COC(=O)OC1CCN(C)CC1]

In an embodiment, an exemplary cationic lipid is RV97 having the following structure:

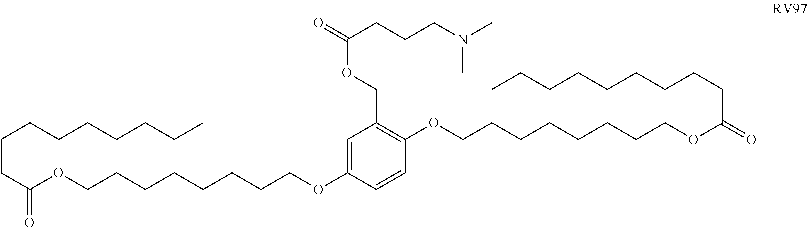 [SMILES: CCCCCCCCCC(=O)OCCCCCCCCOc1ccc(OCCCCCCCCOC(=O)CCCCCCCCC)c(COC(=O)CCCN(C)C)c1]

In an embodiment, an exemplary cationic lipid is RV99 having the following structure:

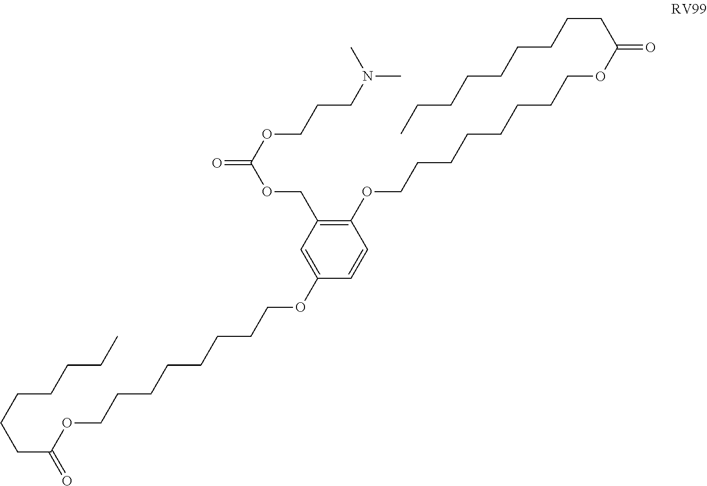 [SMILES: CCCCCCCCCC(=O)OCCCCCCCCOc1ccc(OCCCCCCCCOC(=O)CCCCCCC)cc1COC(=O)OCCCN(C)C]

In an embodiment, an exemplary cationic lipid is RV101 having the following structure:

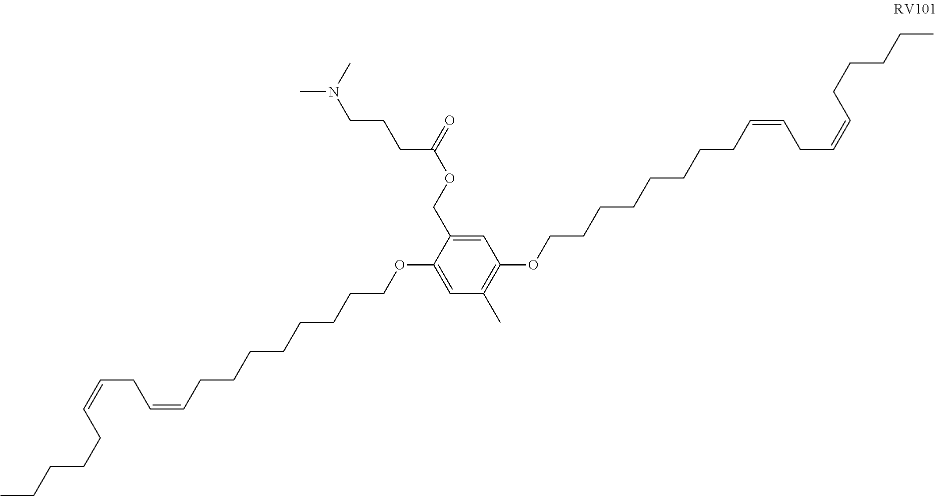 [SMILES: CCCCC/C=C\C/C=C\CCCCCCCCOc1cc(COC(=O)CCCN(C)C)c(OCCCCCCCC/C=C\C/C=C\CCCCC)cc1C]

In an embodiment, the cationic lipid is selected from the group consisting of: RV39, RV88, and RV94.

Compositions and methods for the synthesis of compounds having Formula I and RV28, RV31, RV33, RV37, RV39, RV42, RV44, RV73, RV75, RV81, RV84, RV85, RV86, RV88, RV91, RV92, RV93, RV94, RV95, RV96, RV97, RV99, and RV101 can be found in WO/2015/095340, WO/2015/095346) and WO/2016/037053).

The ratio of RNA to lipid can be varied. The ratio of nucleotide (N) to phospholipid (P) can be in the range of, e.g., 1N:1P, 2N:1P, 3N:1P, 4N:1P, 5N:1P, 6N:1P, 7N:1P, 8N:1P, 9N:1P, or 10N:1P. The ratio of nucleotide (N) to phospholipid (P) can be in the range of, e.g., 1N:1P to 10N:1P, 2N:1P to 8N:1P, 2N:1P to 6N:1P or 3N:1P to 5N:1P.

Alternatively or additionally, the ratio of nucleotide (N) to phospholipid (P) is 4N:1P. Alternatively or additionally, the nucleic acid-based vaccine comprises a cationic nanoemulsion (CNE) delivery system. Cationic oil-in water emulsions can be used to deliver negatively charged molecules, such as RNA molecules, to the interior of a cell. The emulsion particles comprise a hydrophobic oil core and a cationic lipid, the latter of which can interact with the RNA, thereby anchoring it to the emulsion particle. In a CNE delivery system, the nucleic acid molecule (e.g., RNA) which encodes the antigen is complexed with a particle of a cationic oil-in-water emulsion.

Thus, in a nucleic acid-based vaccine of the invention, an RNA molecule encoding an antigen may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. Alternatively or additionally, the CNE comprises squalene and a cationic lipid, such as 1,2-dioleoyloxy-3-(trimethylammonio)propane (DOTAP). In an embodiment, the CNE is an oil in water emulsion of DOTAP and squalene stabilized with polysorbate.

Alternatively or additionally, the process of manufacturing a self-amplifying RNA comprises a step of in vitro transcription (IVT). In some embodiments, the process of manufacturing a self-amplifying RNA comprises a step of IVT to produce an RNA, followed by a capping 5′ dinucleotide m7G(5′)ppp(5′)G reaction and further comprises a step of combining the RNA with a non-viral delivery system. Alternatively or additionally, the process of manufacturing a self-amplifying RNA comprises a step of IVT to produce an RNA, and further comprises a step of combining the RNA with a lipid based delivery system.

The LNP and CNE delivery systems of the invention can be particularly effective in eliciting both humoral and cellular immune responses to antigens expressed by self-amplifying vectors. Advantages of these delivery systems also include the absence of a limiting anti-vector immune response.

Constructs, Antigens and Variants

The present invention provides constructs useful as components of immunogenic compositions for the induction of an immune response in a subject against diseases caused by infectious pathogenic organisms. These constructs are useful for the expression of antigens, methods for their use in treatment, and processes for their manufacture. A “construct” is a genetically engineered molecule. A “nucleic acid construct” refers to a genetically engineered nucleic acid and may comprise RNA or DNA, including non-naturally occurring nucleic acids. In some embodiments, the constructs disclosed herein encode wild-type polypeptide sequences, variants or fragments thereof of pathogenic organisms, e.g., viruses, bacteria, fungi, protozoa or parasite.

A “vector” refers to a nucleic acid that has been substantially altered relative to a wild type sequence and/or incorporates a heterologous sequence, i.e., nucleic acid obtained from a different source, and replicating and/or expressing the inserted polynucleotide sequence, when introduced into a cell (i.e., a “host cell”). In the case of replication defective adenoviruses, the host cell may be E1 complementing.

As used herein, the term “antigen” refers to a molecule containing one or more epitopes (e.g., linear, conformational or both) that will stimulate a host's immune system to make a humoral, i.e., B cell mediated antibody production, and/or cellular antigen-specific immunological response (i.e. T cell mediated immunity). An “epitope” is that portion of an antigen that determines its immunological specificity.

T- and B-cell epitopes can be identified empirically (e.g. using PEPSCAN or similar methods). They can also be predicted by known methods (e.g. using the Jameson-Wolf antigenic index, matrix-based approaches, TEPITOPE, neural networks, OptiMer & EpiMer, ADEPT, Tsites, hydrophilicity or antigenic index.

A “variant” of a polypeptide sequence includes amino acid sequences having one or more amino acid additions, substitutions and/or deletions when compared to the reference sequence. The variant may comprise an amino acid sequence which is at least 70%, at least 75%, at least 80%, at least 85%, at least 90%, at least 95%, at least 96%, at least 97%, at least 98%, or at least 99% identical to a full-length wild-type polypeptide. Alternatively, or in addition to, a fragment of a polypeptide may comprise an immunogenic fragment (i.e. an epitope-containing fragment) of the full-length polypeptide which may comprise or consist of a contiguous amino acid sequence of at least 8, at least 9, at least 10, at least 11, at least 12, at least 13, at least 14, at least 15, at least 16, at least 17, at least 18, at least 20, or more amino acids which is identical to a contiguous amino acid sequence of the full-length polypeptide.

Alternatively or additionally, the cross-protective breadth of a vaccine construct can be increased by comprising a medoid sequence of an antigen. By “medoid” is meant a sequence with a minimal dissimilarity to other sequences. Alternatively or additionally, a vector of the invention comprises a medoid sequence of a protein or immunogenic fragment thereof. Alternatively or additionally, a self-amplifying RNA construct of the invention comprises a medoid sequence of a protein. Alternatively or additionally, the medoid sequence is derived from a natural viral strain with the highest average percent of amino acid identity among all related protein sequences annotated in the NCBI database.

As a result of the redundancy in the genetic code, a polypeptide can be encoded by a variety of different nucleic acid sequences. Coding is biased to use some synonymous codons, i.e., codons that encode the same amino acid, more than others. By “codon optimized” it is meant that modifications in the codon composition of a recombinant nucleic acid are made without altering the amino acid sequence. Codon optimization has been used to improve mRNA expression in different organisms by using organism-specific codon-usage frequencies.

In addition to, and independently from, codon bias, some synonymous codon pairs are used more frequently than others. This codon pair bias means that some codon pairs are overrepresented and others are underrepresented. By “codon pair optimized,” it is meant that modifications in the codon pairing are made without altering the amino acid sequence.

Codon pair deoptimization has been used to reduce viral virulence. For example, it has been reported that polioviruses modified to contain underrepresented codon pairs demonstrated a decreased translation efficiency and were attenuated compared to wild type poliovirus (WO 2008/121992; Coleman et al. (2008) Science 320:1784). Coleman et al. demonstrated that engineering a synthetic attenuated virus by codon pair deoptimization can produce viruses that encode the same amino acid sequences as wild type but use different pairwise arrangements of synonymous codons. Viruses attenuated by codon pair deoptimization generated up to 1000-fold fewer plaques compared to wild type, produced fewer viral particles and required about 100 times as many viral particles to form a plaque.

In contrast, polioviruses modified to contain codon pairs that are overrepresented in the human genome acted in a manner similar to wild type RNA and generated plaques identical in size to wild type RNA (Coleman et al. (2008) Science 320:1784). This occurred despite the fact that the virus with overrepresented codon pairs contained a similar number of mutations as the virus with underrepresented codon pairs and demonstrated enhanced translation compared to wild type.

Alternatively or additionally, a construct of the invention comprises a codon optimized nucleic acid sequence. Alternatively or additionally, an adenoviral or self-amplifying RNA construct of the invention comprises a codon optimized sequence of a protein or an immunogenic derivative or fragment thereof.

Alternatively or additionally, a construct of the invention comprises a codon pair optimized nucleic acid sequence. Alternatively or additionally, a self-amplifying RNA construct of the invention comprises or consists of a codon pair optimized sequence of a protein or an immunogenic derivative or fragment thereof.

Polypeptides

By “polypeptide” is meant a plurality of covalently linked amino acid residues defining a sequence and linked by amide bonds. The term is used interchangeably with “peptide” and “protein” and is not limited to a minimum length of the polypeptide. The term polypeptide also embraces post-translational modifications introduced by chemical or enzyme-catalyzed reactions, as are known in the art. The term can refer to fragments of a polypeptide or variants of a polypeptide such as additions, deletions or substitutions.

Alternatively or additionally, a polypeptide herein is in a non-naturally occurring form (e.g. a recombinant or modified form). Polypeptides of the invention may have covalent modifications at the C-terminus and/or N-terminus. They can also take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.). The polypeptides can be naturally or non-naturally glycosylated (i.e. the polypeptide may have a glycosylation pattern that differs from the glycosylation pattern found in the corresponding naturally occurring polypeptide).

Non-naturally occurring forms of polypeptides herein may comprise one or more heterologous amino acid sequences (e.g. another antigen sequence, another signal sequence, a detectable tag, or the like) in addition to an antigen sequence. For example, a polypeptide herein may be a fusion protein. Alternatively, or in addition, the amino acid sequence or chemical structure of the polypeptide may be modified (e.g. with one or more non-natural amino acids, by covalent modification, and/or or by having a different glycosylation pattern, for example, by the removal or addition of one or more glycosyl groups) compared to a naturally-occurring polypeptide sequence.

Identity with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 or swgapdnamt can be used in conjunction with the computer program. In an embodiment, the gap opening penalty is 15, the gap extension penalty is 6.66, the gap separation penalty range is eight and the percent identity for alignment delay is 40. By way of example, the percent identity can be calculated as the total number of identical matches multiplied by 100 and then divided by the sum of the length of the longer sequence within the matched span and the number of gaps introduced into the shorter sequences in order to align the two sequences.

Where the present disclosure refers to a sequence by reference to a UniProt or GenBank accession code, the sequence referred to is the current version as of the filing date of the present application.

The skilled person will recognise that individual substitutions, deletions or additions to a protein which alters, adds or deletes a single amino acid or a small percentage of amino acids is an “immunogenic derivative” where the alteration(s) results in the substitution of an amino acid with a functionally similar amino acid or the substitution/deletion/addition of residues which do not impact the immunogenic function.

Conservative substitution tables providing functionally similar amino acids are well known in the art. In general, such conservative substitutions will fall within one of the amino-acid groupings specified below, though in some circumstances other substitutions may be possible without substantially affecting the immunogenic properties of the antigen. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

Suitably such substitutions do not occur in the region of an epitope, and do not therefore have a significant impact on the immunogenic properties of the antigen.

Immunogenic derivatives may also include those wherein additional amino acids are inserted compared to the reference sequence. Suitably such insertions do not occur in the region of an epitope, and do not therefore have a significant impact on the immunogenic properties of the antigen. One example of insertions includes a short stretch of histidine residues (e.g. 2-6 residues) to aid expression and/or purification of the antigen in question.

Immunogenic derivatives include those wherein amino acids have been deleted compared to the reference sequence. Suitably such deletions do not occur in the region of an epitope, and do not therefore have a significant impact on the immunogenic properties of the antigen. The skilled person will recognise that a particular immunogenic derivative may comprise substitutions, deletions and additions (or any combination thereof).

Transgenes

Adenoviruses or RNA molecules may be used to deliver desired RNA or protein sequences, for example heterologous sequences, for in vivo expression. A vector comprising a gene of interest of the invention may include any genetic element, including DNA, RNA, a phage, transposon, cosmid, episome, plasmid or viral component. Vectors of the invention may contain simian adenoviral DNA and an expression cassette. An “expression cassette” comprises a transgene and regulatory elements necessary for the translation, transcription and/or expression of the transgene in a host cell.

A “transgene” is a nucleic acid sequence, heterologous to the vector sequences flanking the transgene, which encodes a polypeptide of interest. “Transgene” and “immunogen” are used interchangeably herein. The nucleic acid coding sequence is operatively linked to regulatory components in a manner which permits transgene transcription, translation, and/or expression in a host cell. In embodiments of the invention, the vectors express transgenes at a therapeutic or a prophylactic level. A “functional derivative” of a transgenic polypeptide is a modified version of a polypeptide, e.g., wherein one or more amino acids are deleted, inserted, modified or substituted.

The transgene may be used for prophylaxis or treatment, e.g., as a vaccine for inducing an immune response, to correct genetic deficiencies by correcting or replacing a defective or missing gene, or as a cancer therapeutic. As used herein, “inducing an immune response” refers to the ability of a protein to induce a T cell and/or a humoral antibody immune response to the protein.

The composition of the transgene sequence will depend upon the use to which the resulting vector will be put. In an embodiment, the transgene is a sequence encoding a product which is useful in biology and medicine, such as a prophylactic transgene, a therapeutic transgene or an immunogenic transgene, e.g., protein or RNA. Protein transgenes include antigens. Antigenic transgenes of the invention induce an immunogenic response to a disease causing organism. RNA transgenes include tRNA, dsRNA, ribosomal RNA, catalytic RNAs, and antisense RNAs. An example of a useful RNA sequence is a sequence which extinguishes expression of a targeted nucleic acid sequence in the treated animal.

In addition to the transgene, the expression cassette also includes conventional control elements which are operably linked to the transgene in a manner that permits its transcription, translation and/or expression in a cell transfected with the adenoviral vector. As used herein, “operably linked” sequences include both expression control sequences that are contiguous with the gene of interest and expression control sequences that act in trans or at a distance to control the gene of interest.

The immune response elicited by the transgene may be an antigen specific B cell response, which produces neutralizing antibodies. The elicited immune response may be an antigen specific T cell response, which may be a systemic and/or a local response. The antigen specific T cell response may comprise a CD4+ helper T cell response, such as a response involving CD4+ T cells expressing cytokines, e.g. IFN-γ (IFN-γ), tumor necrosis factor alpha (TNF-α) and/or interleukin 2 (IL2). Alternatively, or additionally, the antigen specific T cell response comprises a CD8+ cytotoxic T cell response, such as a response involving CD8+ T cells expressing cytokines, e.g., IFN-γ, TNF-α and/or IL2.

An “immunologically effective amount” is the amount of an active component sufficient to elicit either an antibody or a T cell response or both sufficient to have a beneficial effect, e.g., a prophylactic or therapeutic effect, on the subject.

A transgene sequence may include a reporter sequence, which upon expression produces a detectable signal. Such reporter sequences include, without limitation, DNA sequences encoding beta-lactamase, beta-galactosidase (LacZ), alkaline phosphatase, thymidine kinase, green fluorescent protein (GFP), chloramphenicol acetyltransferase (CAT), luciferase, membrane bound proteins including, for example, CD2+, CD4+, CD8+, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry.

A construct of the invention may comprise a codon optimized nucleic acid sequence as a transgene. Alternatively or additionally, a vector of the invention may comprise a codon optimized sequence of a transgene or an immunogenic derivative or fragment thereof. A construct of the invention may comprise a codon pair optimized nucleic acid sequence as a transgene. Alternatively or additionally, a vector of the invention may comprise a codon pair optimized sequence of a transgene or an immunogenic derivative or fragment thereof.

If desired, the adenovirus and self-amplifying RNA molecules can be screened or analyzed to confirm their therapeutic and prophylactic properties using various in vitro or in vivo testing methods that are known to those of skill in the art. For example, ELISA assays can measure immunoglobulin levels specific to the transgenic antigen. A Fluorescent Antibody Virus Neutralization test (FAVN) can measure the level of virus neutralizing activity by antibodies induced by the antigen. Vaccines of the invention can be tested for their effect on the induction of proliferation or on the effector function of a particular lymphocyte type of interest, e.g., B cells, T cells, T cell lines or T cell clones. For example, spleen cells from immunized mice can be isolated and the capacity of cytotoxic T lymphocytes to lyse autologous target cells that contain a self-amplifying RNA molecule encoding an antigen. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry. Antigen specific T cells can be measured by methods known in the art, e.g., pentamer staining assays.

Adenovirus and self-amplifying RNA molecules that encode an antigen can also be tested for their ability to induce humoral immune responses, as evidenced, for example, by induction of B cell production of antibodies specific for an antigen of interest. These assays can be conducted using, for example, peripheral B lymphocytes from immunized individuals. Such assay methods are known to those of skill in the art. Other assays that can be used to characterize the vectors of the invention involve detecting expression of the encoded antigen by the target cells. For example, fluorescent activated cell sorting (FACS) can be used to detect antigen expression on the cell surface or intracellularly. Another advantage of FACS selection is that one can sort for different levels of expression, as sometimes a lower expression may be desired. Other suitable methods for identifying cells which express a particular antigen involve panning using monoclonal antibodies on a plate or capture using magnetic beads coated with monoclonal antibodies.

### Pharmaceutical Compositions, Immunogenic Compositions

The invention provides compositions comprising a nucleic acid comprising a sequence which encodes a polypeptide, for example an antigen. The composition may be a pharmaceutical composition, e.g., an immunogenic composition or a vaccine composition. The composition may comprise an adenovirus or a SAM. Accordingly, the composition may also comprise a pharmaceutically acceptable carrier.

A “pharmaceutically acceptable carrier” includes any carrier that does not itself induce the production of antibodies harmful to the individual receiving the composition. The compositions of the invention may also contain a pharmaceutically acceptable diluent, such as water, sterile pyrogen-free water, saline, phosphate-buffered physiologic saline, glycerol, etc. Additionally, auxiliary substances, such as wetting or emulsifying agents, pH buffering substances, and the like, may be present.

Pharmaceutical compositions may include the constructs, nucleic acid sequences, and/or polypeptide sequences described elsewhere herein in plain water (e.g. water for injection (w.f.i.)) or in a buffer e.g. a phosphate buffer, a Tris buffer, a borate buffer, a succinate buffer, a histidine buffer, or a citrate buffer. Buffer salts will typically be included in the 5-20 mM range. Pharmaceutical compositions may have a pH between 5.0 and 9.5. Compositions may include sodium salts, e.g. sodium chloride, to give tonicity. A concentration of 10±2 mg/ml NaCl is typical, e.g. about 9 mg/ml. Compositions may include metal ion chelators. These can prolong RNA stability by removing ions which can accelerate phosphodiester hydrolysis and contribute to adenovector vector stability. Thus, a composition may include one or more of EDTA, EGTA, BAPTA, pentetic acid, etc. Such chelators are typically present at between 10-500 μM, e.g., 0.1 mM. A citrate salt, such as sodium citrate, can also act as a chelator, while advantageously also providing buffering activity.

Pharmaceutical compositions may have an osmolality of between 200 mOsm/kg and 400 mOsm/kg, e.g. between 240-360 mOsm/kg, or between 290-310 mOsm/kg. Pharmaceutical compositions may include one or more preservatives, such as thiomersal or 2-phenoxyethanol. Mercury-free compositions are preferred, and preservative-free vaccines can be prepared. Pharmaceutical compositions may be aseptic or sterile. Pharmaceutical compositions may be non-pyrogenic e.g. containing <1 EU (endotoxin unit) per dose, and preferably <0.1 EU per dose. Pharmaceutical compositions may be gluten free. Pharmaceutical compositions may be prepared in unit dose form. Alternatively or additionally, a unit dose may have a volume of between 0.1-2.0 ml, e.g. about 1.0 or 0.5 ml.

A composition of the invention may be administered with or without an adjuvant. Alternatively or additionally, the composition may comprise, or be administered in conjunction with, one or more adjuvants (e.g. vaccine adjuvants).

By “adjuvant” is meant an agent that augments, stimulates, activates, potentiates or modulates the immune response to an active ingredient of the composition. The adjuvant effect may occur at the cellular or humoral level or both. Adjuvants stimulate the response of the immune system to the actual antigen but have no immunological effect themselves. Alternatively or additionally, adjuvented compositions of the invention may comprise one or more immunostimulants. By “immunostimulant” it is meant an agent that induces a general, temporary increase in a subject's immune response, whether administered with the antigen or separately.

Methods of Use/Uses

Methods are provided for inducing an immune response against a pathogenic organism in a subject in need thereof comprising a step of administering an immunologically effective amount of a construct or composition as disclosed herein. Some embodiments provide the use of the constructs or compositions disclosed herein for inducing an immune response to an antigen in a subject in need thereof. Some embodiments provide the use of the construct or composition as disclosed herein in the manufacture of a medicament inducing an immune response to an antigen in a subject.

By “subject” is meant a mammal, e.g. a human or a veterinary mammal. In some embodiments the subject is human.

By “priming” is meant the administration of an immunogenic composition which induces a higher level of an immune response, when followed by a subsequent administration of the same or of a different immunogenic composition, than the immune response obtained by administration with a single immunogenic composition.

By “boosting” is meant the administration of a subsequent immunogenic composition after the administration of a priming immunogenic composition, wherein the subsequent administration produces a higher level of immune response than an immune response to a single administration of an immunogenic composition.

By “heterologous prime boost” is meant priming the immune response with an antigen and subsequent boosting of the immune response with an antigen delivered by a different molecule and/or vector. For example, heterologous prime boost regimens of the invention include priming with an RNA molecule and boosting with an adenoviral vector as well as priming with an adenoviral vector and boosting with an RNA molecule.

Routes of Administration

Compositions disclosed herein will generally be administered directly to a subject. Direct delivery may be accomplished by parenteral administration, e.g. buccal, inhalation, intramuscular, intranasal, intraperitoneal, intrathecal, intravenous, oral, rectal, sublingual, transdermal, vaginal or to the interstitial space of a tissue.

As used herein, administration of a composition “subsequently to” administration of a composition indicates that a time interval has elapsed between administration of a first composition and administration of a second composition, regardless of whether the first and second compositions are the same or different.

The amount administered, and the rate and time-course of administration will depend on the nature and severity of what is being treated. Prescription of treatment, e.g., decisions regarding dosage, etc., is within the expertise of general practitioners and other doctors and health care providers. It typically takes into account the condition to be prevented or treated, the method of administration and other factors known to practitioners.

Kits

The invention provides a pharmaceutical kit for the ready administration of an immunogenic, prophylactic or therapeutic regimen for treating a disease or condition caused by a pathogenic organism. The kit is designed for use in a method of inducing an immune response by administering a priming vaccine comprising an immunologically effective amount of one or more antigens encoded by either an adenoviral vector or an RNA molecule and subsequently administering a boosting vaccine comprising an immunologically effective amount of one or more antigens encoded by either an adenoviral vector or an RNA molecule.

The kit contains at least one immunogenic composition comprising an adenoviral vector encoding an antigen and at least one immunogenic composition comprising an RNA molecule encoding an antigen. The kit may contain multiple prepackaged doses of each of the component vectors for multiple administrations of each. Components of the kit may be contained in vials.

The invention provides a pharmaceutical kit for the ready administration of an immunogenic, prophylactic or therapeutic regimen for treating a disease or condition caused by an infectious pathogenic organism. The kit is designed for use in a method of inducing an immune response by administering a priming vaccine comprising an immunologically effective amount of one or more antigens encoded by either a simian adenoviral vector or an RNA molecule and subsequently administering a boosting vaccine comprising an immunologically effective amount of one or more antigens encoded by either a simian adenoviral vector or an RNA molecule.

The kit contains at least one immunogenic composition comprising a simian adenoviral vector encoding an antigen and at least one immunogenic composition comprising an RNA molecule encoding an antigen. The kit may contain multiple prepackaged doses of each of the component vectors for multiple administrations of each. Components of the kit may be contained in vials.

The kit also contains instructions for using the immunogenic compositions in the prime/boost methods described herein. It may also contain instructions for performing assays relevant to the immunogenicity of the components. The kit may also contain excipients, diluents, adjuvants, syringes, other appropriate means of administering the immunogenic compositions or decontamination or other disposal instructions.

Vectors of the invention are generated using techniques and sequences provided herein, in conjunction with techniques known to those of skill in the art. Such techniques include conventional cloning techniques of cDNA such as those described in texts, use of overlapping oligonucleotide sequences of the adenovirus genomes, polymerase chain reaction, and any suitable method which provides the desired nucleotide sequence.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” in relation to a numerical value is optional and means, e.g., the amount ±10%.

The term “comprising” encompasses “including” as well as “consisting,” e.g., a composition comprising X may consist exclusively of X or may include something additional, e.g., X+Y. The term “substantially” does not exclude “completely.” For example, a composition that is substantially free from Z may be completely free from Z.

The invention is further exemplified in the following embodiments.
a. A vaccine combination comprising a first composition comprising an immunologically effective amount of at least one adenovirus vector encoding at least one antigen and a second composition comprising an immunologically effective amount of at least one RNA molecule encoding at least one antigen wherein one of the compositions is a priming composition and the other composition is a boosting composition.
b. The composition of (a) wherein the vaccine combination is effective for prophylaxis or therapy of an infectious condition in a mammalian subject.
c. The composition of (b) wherein the vaccine combination is not used to prevent or treat cancer.
d. Use of the composition of (a) or (b) for the prophylaxis or therapy of an infectious condition in a human.
e. The use of the composition of (a) or (b) in the manufacture of a medicament for an infectious condition.
f. A method of inducing an immune response to an infectious disease in a mammal comprising
   i. administering a priming vaccine comprising an immunologically effective amount of one or more antigens encoded by either an adenoviral vector or an RNA molecule and
   ii. administering a booster vaccine comprising an immunologically effective amount of one or more antigens encoded by either an adenoviral vector or an RNA molecule,
   wherein if the priming vaccine is encoded by an adenoviral vector the booster vaccine is encoded by an RNA molecule, and if the priming vaccine is encoded by an RNA molecule the booster vaccine is encoded by an adenoviral vector.
g. The method or use of any of (d)-(f) wherein the priming vaccine comprises an immunologically effective amount of one or more antigens encoded by an adenoviral vector and the boosting vaccine comprises an immunologically effective amount of one or more antigens encoded by an RNA molecule.
h. The method or use of any of (d)-(g) wherein the priming vaccine comprises an immunologically effective amount of one or more antigens encoded by an RNA molecule and the boosting vaccine comprises an immunologically effective amount of one or more antigens encoded by an adenoviral vector.
i. The method or use of any of (d)-(h) wherein the one or more antigens are from the same pathogenic organism.
j. The method or use of any of (d)-(i) wherein the one or more antigens are the same in the priming vaccine and the boosting vaccine.
k. The method or use of any of (d)-(j) wherein at least one of the epitopes of the one or more antigens are different in the priming and the boosting vaccine.
l. The method or use of any of (d)-(k) wherein the adenoviral vector is a simian adenoviral vector.
m. The method or use of (l) wherein the simian adenoviral vector is selected from a chimpanzee, bonobo, rhesus macaque, orangutan and gorilla vector.
n. The method or use of (m) wherein the simian adenoviral vector is a chimpanzee vector.
o. The method of (n) wherein the chimpanzee vector is selected from AdY25, ChAd3, ChAd15, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, ChAd157, ChAdOx1, ChAdOx2, SadV41, sAd4287, sAd4310A, sAd4312, SAdV31 and SAdV-A1337.
p. The method or use of any of (d)-(o) wherein the RNA molecule is a messenger RNA (mRNA) molecule.
q. The method or use of (p) wherein the mRNA molecule is a self-amplifying RNA vector.
r. The method or use of any of (d)-(q) wherein the antigen is encoded in an adenoviral vector comprising an expression cassette comprising a transgene and regulatory elements necessary for the translation, transcription and/or expression of the transgene in a host cell.
s. The method or use of (r) wherein the antigen is a polypeptide antigen.
t. The method or use of any of (d)-(s), wherein the RNA molecule is delivered as a cationic nanoemulsion (CNE) or a lipid nanoparticle (LNP).
u. The method or use of (t), wherein the LNP comprises a cationic lipid selected from the group consisting of:

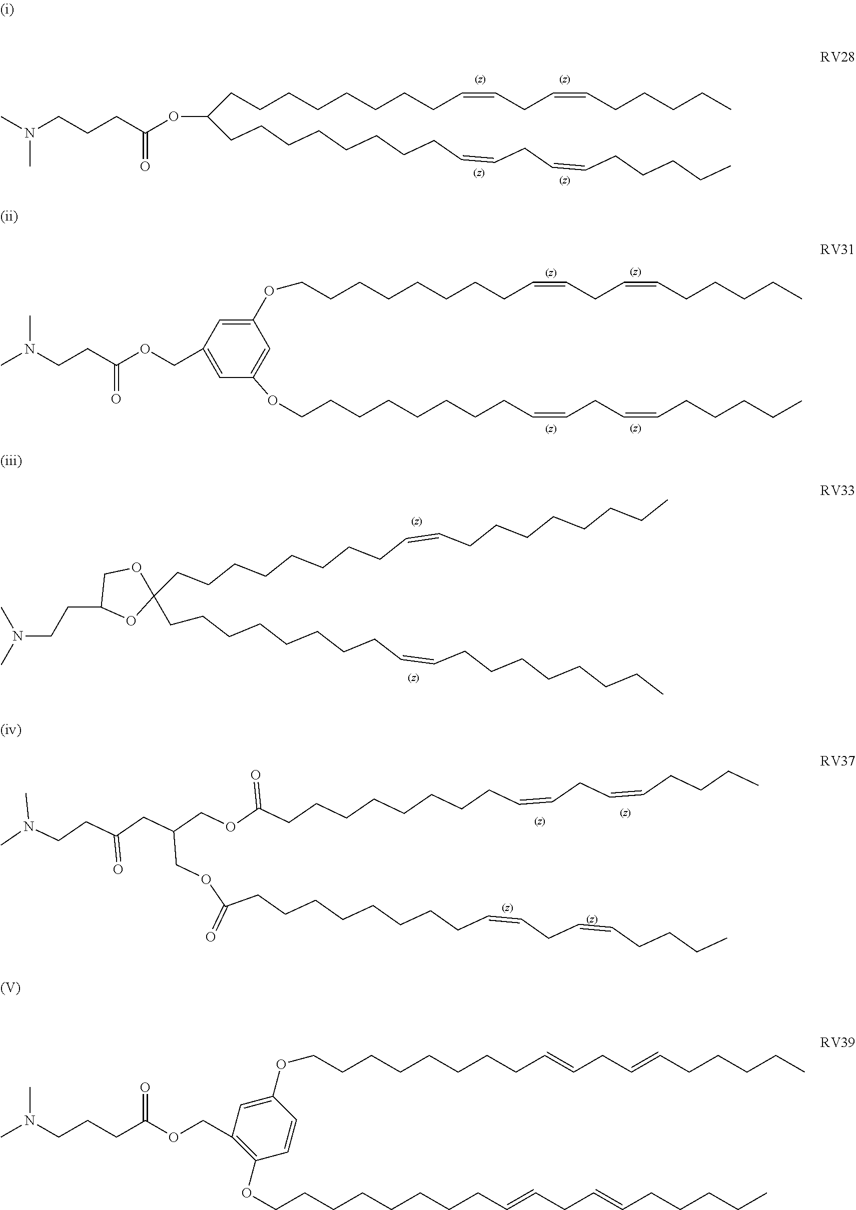

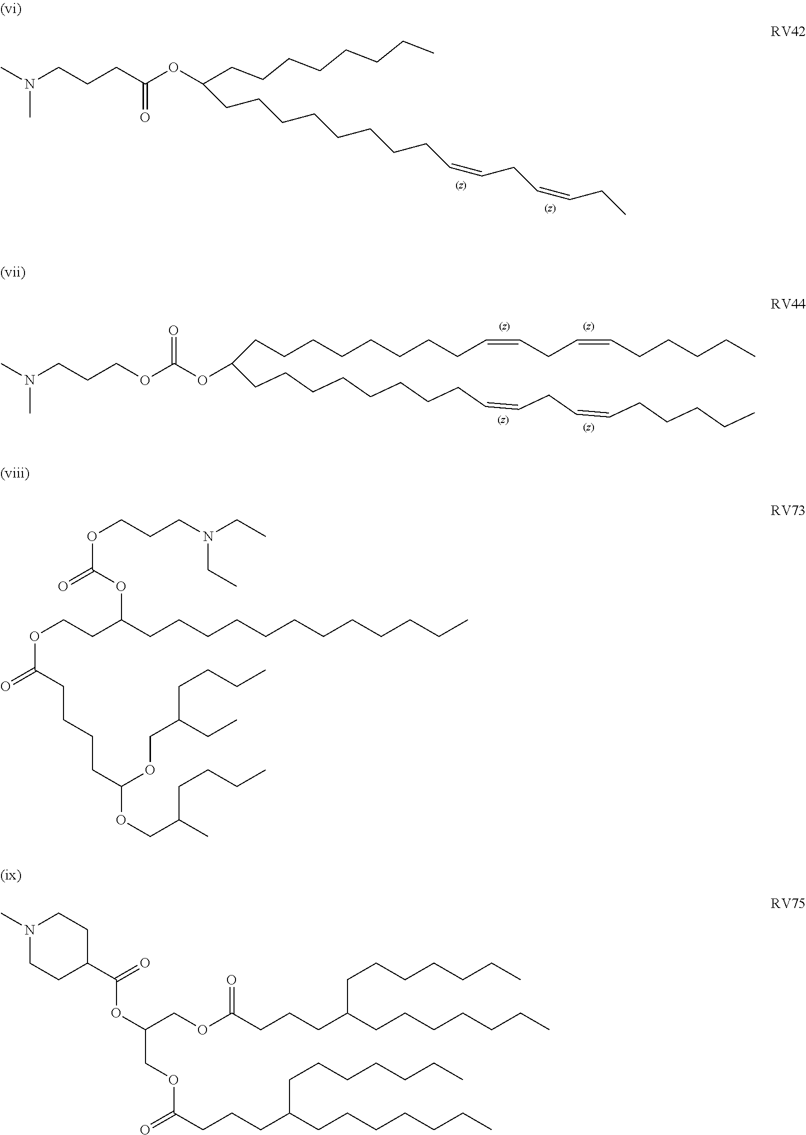

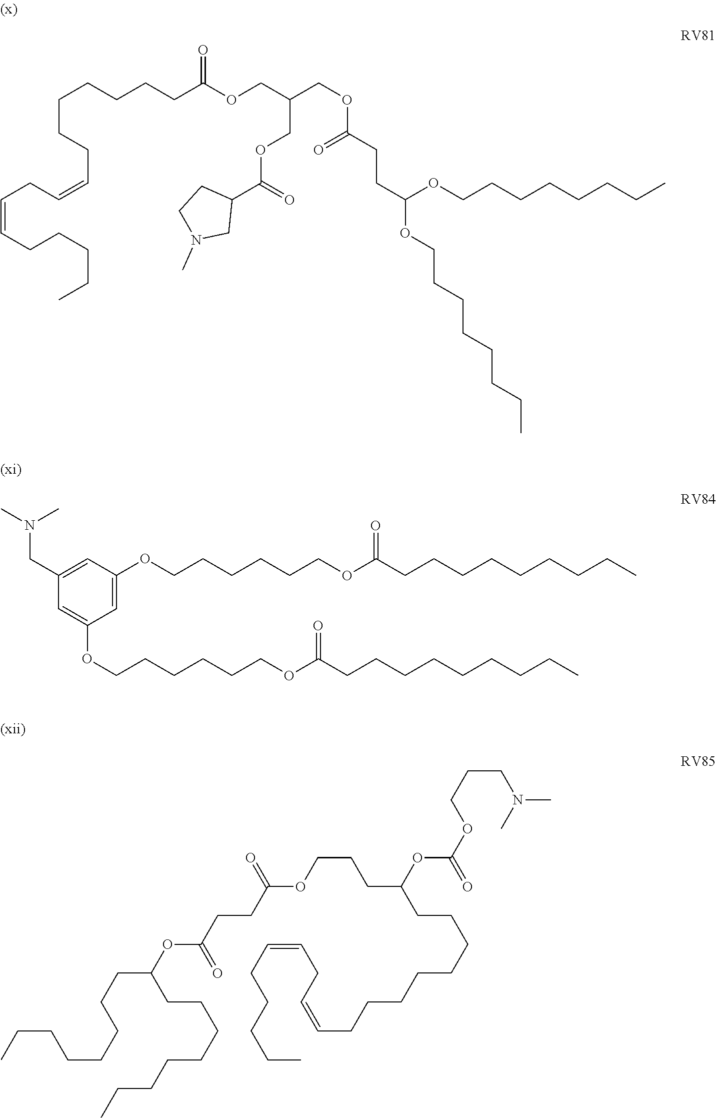

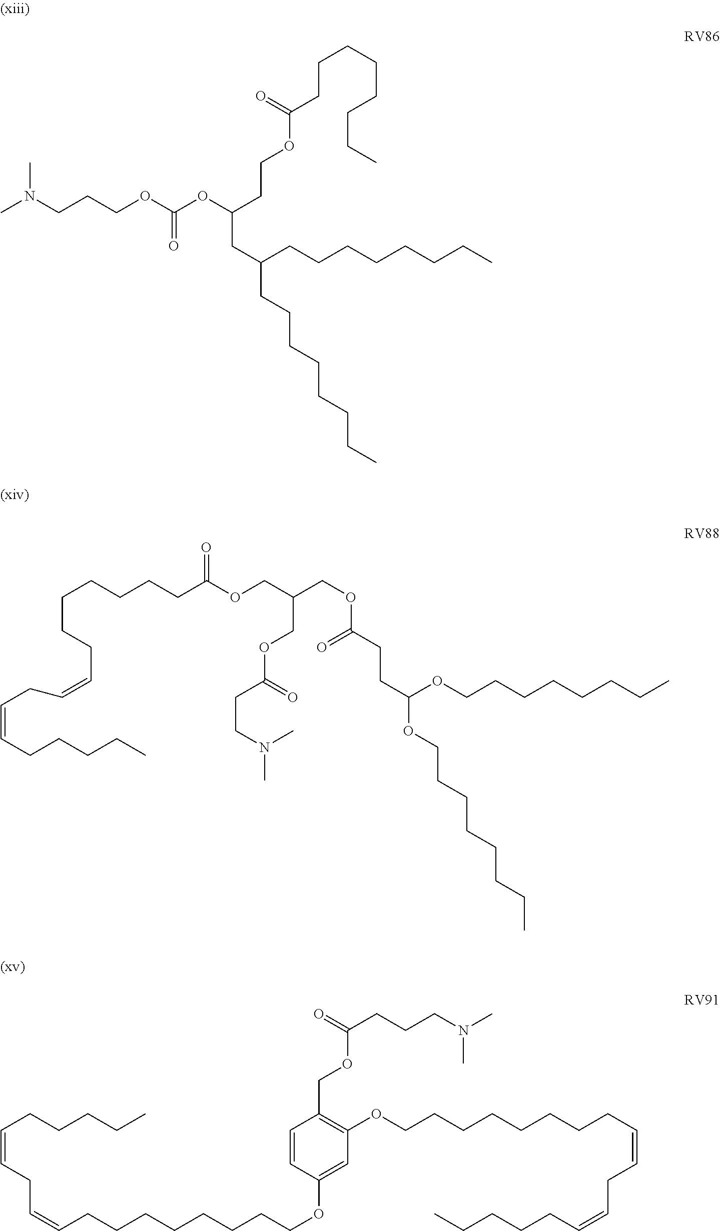 [SMILES: CCCCCCCCC(=O)OCCC(CC(CCCCCCCC)CCCCCCCC)OC(=O)OCCCN(C)C ; CCCCC/C=C\C/C=C\CCCCCCCC(=O)OCC(COC(=O)CCC(OCCCCCCCC)OCCCCCCCC)COC(=O)CCN(C)C ; CCCCC/C=C\C/C=C\CCCCCCCCOc1ccc(COC(=O)CCCN(C)C)c(OCCCCCCCC/C=C\C/C=C\CCCCC)c1]

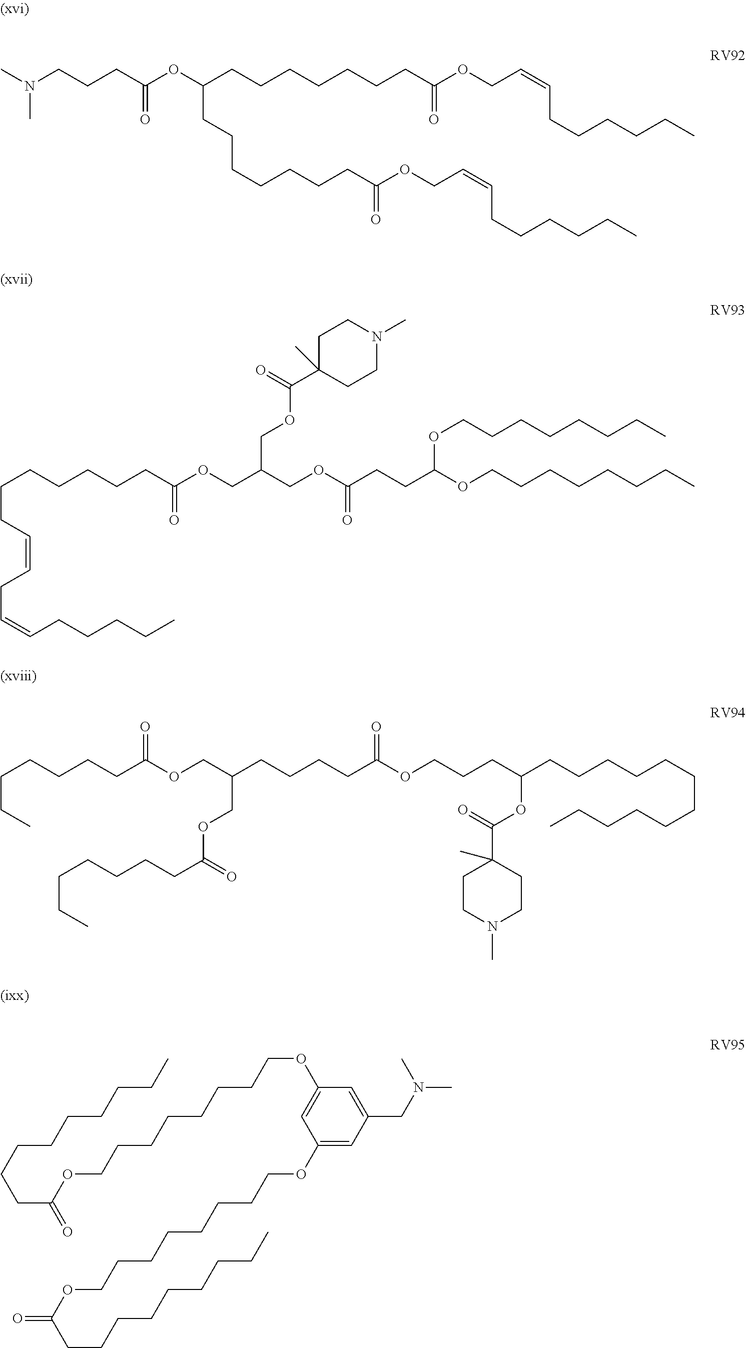 [SMILES: CCCCCC/C=C\COC(=O)CCCCCCCC(CCCCCCCC(=O)OC/C=C\CCCCCC)OC(=O)CCCN(C)C ; CCCCC/C=C\C/C=C\CCCCCCCC(=O)OCC(COC(=O)CCC(OCCCCCCCC)OCCCCCCCC)COC(=O)C1(C)CCN(C)CC1 ; CCCCCCCCCCCCC(CCCOC(=O)CCCCC(COC(=O)CCCCCCC)COC(=O)CCCCCCC)OC(=O)C1(C)CCN(C)CC1 ; CCCCCCCCCC(=O)OCCCCCCCCOc1cc(CN(C)C)cc(OCCCCCCCCOC(=O)CCCCCCCCC)c1]

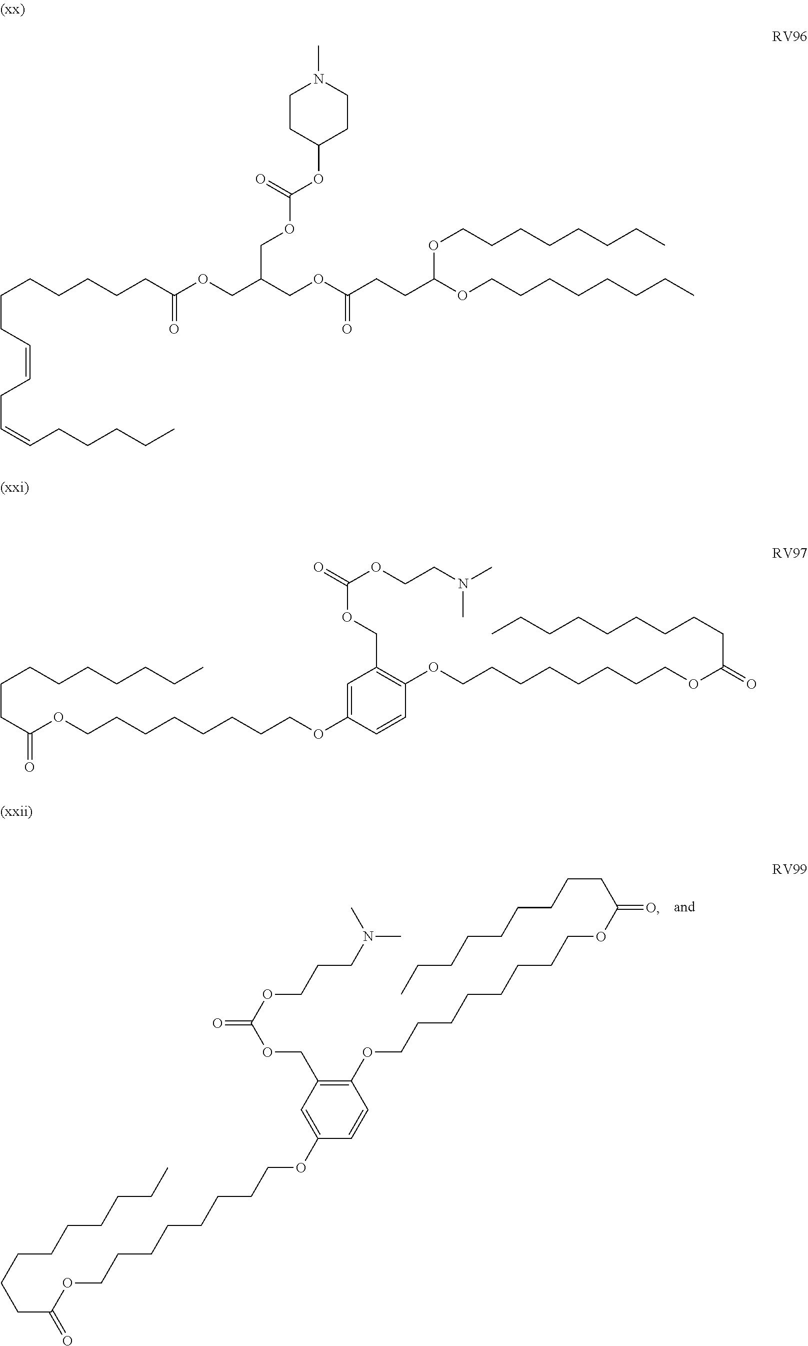 [SMILES: CCCCC/C=C\C/C=C\CCCCCCCC(=O)OCC(COC(=O)CCC(OCCCCCCCC)OCCCCCCCC)COC(=O)OC1CCN(C)CC1 ; CCCCCCCCCC(=O)OCCCCCCCCOc1ccc(OCCCCCCCCOC(=O)CCCCCCCCC)c(COC(=O)OCCN(C)C)c1 ; CCCCCCCCCC(=O)OCCCCCCCCOc1ccc(OCCCCCCCCOC(=O)CCCCCCCCC)c(COC(=O)OCCCN(C)C)c1]

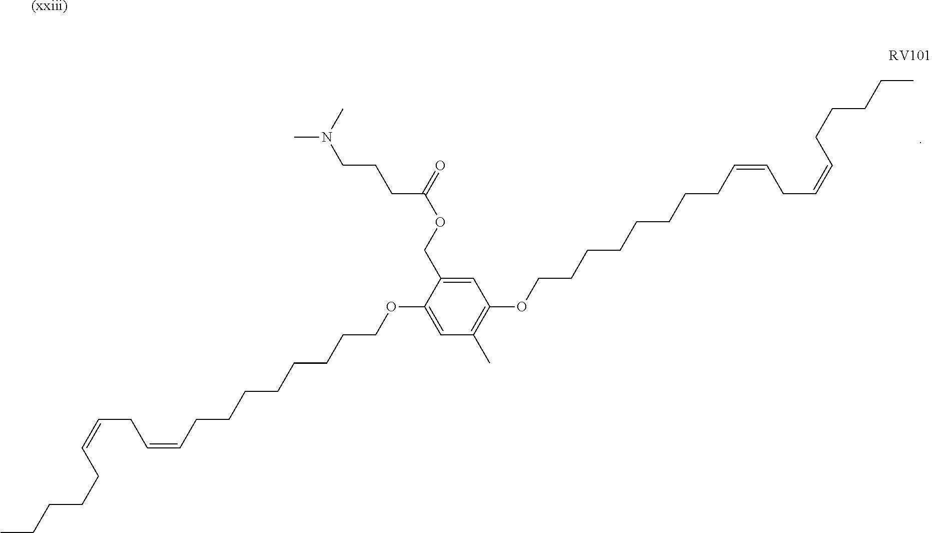 [SMILES: "(xxiii)" ; CCCCC/C=C\C/C=C\CCCCCCCCOc1cc(COC(=O)CCCN(C)C)c(OCCCCCCCC/C=C\C/C=C\CCCCC)cc1C]

v. The method or use of any of (d)-(u) wherein the immune response is an antibody response.
w. The method or use of any of (d)-(u) wherein the immune response is a T cell response.
x. The method or use of any of (d)-(w) wherein at least one of the priming and boosting immunogenic compositions comprises an adjuvant.
y. A priming vaccine comprising an immunologically effective amount of an antigen encoded by either an adenoviral vector or an RNA molecule followed by a boosting vaccine comprising an immunologically effective amount of an antigen encoded by either an adenoviral vector or an RNA molecule for use in preventing or treating a disease caused by an infectious pathogenic organism, wherein if the priming vaccine is encoded by an adenoviral vector, the booster vaccine is encoded by an RNA molecule, and if the priming vaccine is encoded by an RNA molecule the booster vaccine is encoded by an adenoviral vector.
z. A kit according to (a)-(c) or (y) for a prime boost administration regimen comprising at least two vials, the first vial containing a vaccine for the priming administration and the second vial containing a vaccine for the boosting administration.

The present invention will now be further described by means of the following non-limiting examples.

### EXAMPLES

The Examples set forth below describe immunogenic prime boost regimens using three model antigens (rabies glycoprotein, HIV1-GAG and HSV Gly VI) to characterize the kinetics and magnitude of the immune response elicited by adenovirus and RNA vaccines. These antigens were chosen as examples of different categories of antigens to demonstrate the universality of adenoviral/RNA prime boost combinations. The rabies G protein is an example of an envelope glycoprotein, HIV GAG is an example of a viral capsid protein and HSV Gly IV is an example of an artificial fusion polyantigen. The following examples demonstrate that simian adenovirus and small amounts of self-amplifying RNA can be combined in heterologous prime/boost regimens to elicit humoral and cellular immune responses to a wide range of encoded antigens.

### Example 1: Rabies Glycoprotein (RG) as a Model Antigen for a Prime Boost Regimen

Simian adenoviral vectors encoding a codon pair optimized rabies glycoprotein (RG) antigen transgene sequence (WO 2018/104919) were cloned and used to prepare adenoviral particles in chimpanzee adenovirus 155 (ChAd155). Self-amplifying RNA vectors encoding the codon pair optimized rabies glycoprotein antigen sequence were cloned and used to prepare in vitro transcribed capped RNA (SAM-RG).

Adenoviral vectors (ChAd-RG) and self-amplifying RNA (SAM-RG) were each characterized for in vitro potency and formulated for vaccine injection in mice.

Adenoviral vectors were formulated in 10 mM Tris pH 7.4, 10 mM histidine, 75 mM NaCl, 5% sucrose, 0.02% polysorbate 80, 0.1 mM EDTA, 1 mM MgCl₂ (“Tris-NaCl”). SAM-RG was formulated in either a cationic nanoemulsion (CNE); or as lipid nanoparticles (LNP) with RV39 as the lipid.

### Experiment 1: Single Administration of Rabies Antigen

Six week old female BALB/c mice were allocated into groups of ten and the adenoviral or SAM vectors were administered intramuscularly according to the regimens shown in the table below. Adenovirus was administered at doses of 10⁸ and 107 viral particles (vp). RNA was administered in doses of 0.015-15 ug. The animals were bled at weeks 2, 4, 6 and 8 for antibody analysis and weeks 3, 6 and 8 for an analysis of the T cells in the circulating bloodstream. They were sacrificed at week 8, when the spleens were collected to determine T cell functionality.

| Group | Antigen | Vector | Formulation | Dose | |
|---|---|---|---|---|---|
| 1 | Rabies G protein | Adenovirus | Tris-NaCl | 10⁸ | vp |
| 2 | Rabies G protein | Adenovirus | Tris-NaCl | 10⁷ | vp |
| 3 | Rabies G protein | RNA | LNP | 1.5 | ug |
| 4 | Rabies G protein | RNA | LNP | 0.015 | ug |
| 5 | Rabies G protein | RNA | CNE | 15 | ug |
| 6 | Rabies G protein | RNA | CNE | 1.5 | ug |

An analysis of rabies specific humoral and cellular immune responses was performed on samples taken in the eight weeks post-immunization. Rabies virus neutralizing antibody (VNA) titer was measured by a standard, WHO approved Fluorescent Antibody Virus Neutralization (FAVN) assay. Titers above 0.5 IU/ml are considered protective.

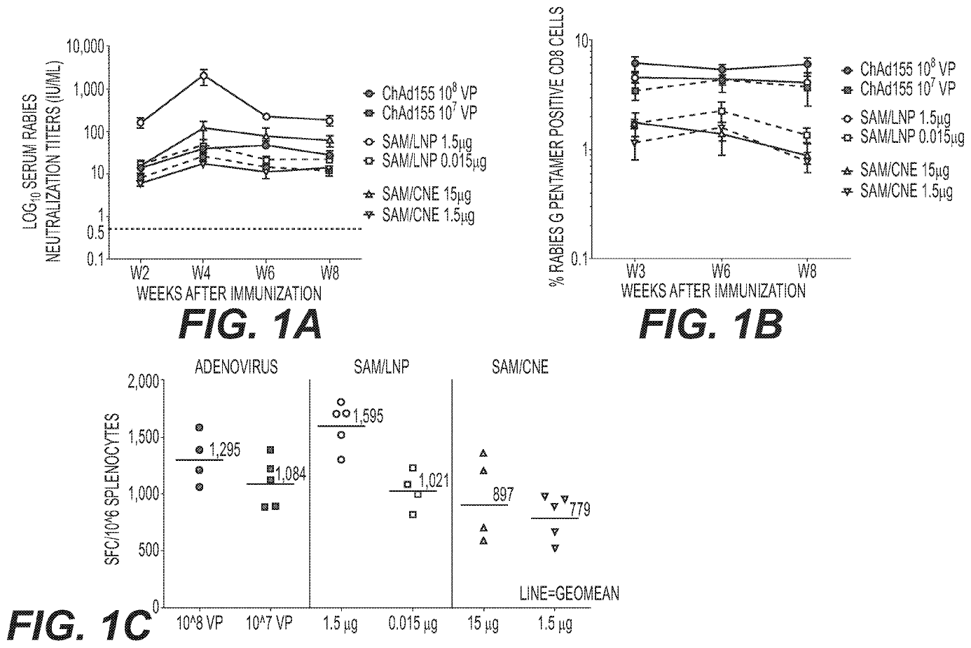

FIG. 1 shows the antibody immune response after one dose of either adenovirus or RNA encoding RG. Both vaccines induced high levels of neutralizing antibody titers, expressed in IU/ml (FIG. 1A). Both vaccines elicited stronger responses at higher doses, with all titers peaking at about four weeks post vaccination, then slightly contracting and stabilizing.

The CD8+ T cell response was quantified with a flow cytometry based staining assay after binding to a pentamer specific for RG antigen. The pentamer consisted of the Major Histocompatibility Complex I H-2 Ld-restricted LPNWGKYVL RG antigen immunodominant CD8 epitope and was conjugated with an allophycocyanin (APC) fluorochrome, to allow quantification of antigen-specific T cells. Peripheral whole blood comprising RG antigen specific T cells in was incubated with the APC-pentamer and fluorochorme labelled antibodies to T cell markers. After washing steps, positive cells were quantified by flow cytometry. Results are expressed as the percentage of CD8+ T cells that were RG-antigen specific, i.e., positive for pentamer staining.

FIG. 1B demonstrates that both the adenovirus and the SAM rabies vaccines elicited strong CD8+ T cell responses to the RG antigen in a dose-dependent manner at all doses and formulations tested.

The functional T cell responses were then measured in splenocytes by IFNγ ELISpot using pools of overlapping 15mer peptides encompassing the entire RG protein amino acid sequence for stimulation (FIG. 1C). IFNγ ELISpot analysis allows enumeration of antigen specific T cells that secrete the cytokine using a sandwich of a capture antibody to IFN-γ bound to a membrane and a complex of a marker biotinylated Ab and streptavidin conjugated to the alkaline phosphatase enzyme, resulting in the precipitation of a chromogenic substrate that generates a spot on the membrane where the antigen specific cell was located. Evaluation of splenocytes at week eight confirmed that both vaccines elicited strong functional T cell responses, i.e., the T cells secreted cytokine in response to the RG antigen, in a dose-dependent manner (FIG. 1C).

Prime/Boost with Rabies Antigen

Based on the single administration data, the priming doses of 107 vp ChAd-RG; 0.015 μg SAM/LNP; and 15 μg SAM/CNE were selected for prime/boost regimen as the lowest effective doses able to confer immunogenicity levels that were comparable between the adenovirus-RG and the RNA-RG vaccines after priming. The interval between prime and boost was eight weeks.

Female BALB/c mice, six weeks of age, were allocated into groups of ten and the adenoviruses or RNA molecules were administered intramuscularly in regimens shown in the table below. The animals were bled at 2, 4 and 8 weeks after the priming and at 2, 4 and 8 weeks after the boosting dose; then sacrificed at week 16, when the spleens were collected to determine T cell functionality. Serology for neutralizing antibodies and T cell assays were performed as with the single administration.

| | Priming | Priming | | Boosting | Boosting | |
|---|---|---|---|---|---|---|
| Group | Vector | Dose | | Vector | Dose | |
| 1 | Adenovirus | 10⁷ | vp | Adenovirus | 10⁷ | vp |
| 2 | Adenovirus | 10⁷ | vp | RNA in LNP | 0.015 | ug |
| 3 | Adenovirus | 10⁷ | vp | RNA in CNE | 15 | ug |
| 4 | RNA in LNP | 0.015 | ug | RNA in LNP | 0.015 | g |
| 5 | RNA in LNP | 0.015 | ug | Adenovirus | 10⁷ | vp |
| 6 | RNA in CNE | 15 | ug | RNA in CNE | 15 | ug |
| 7 | RNA in CNE | 15 | ug | Adenovirus | 10⁷ | vp |

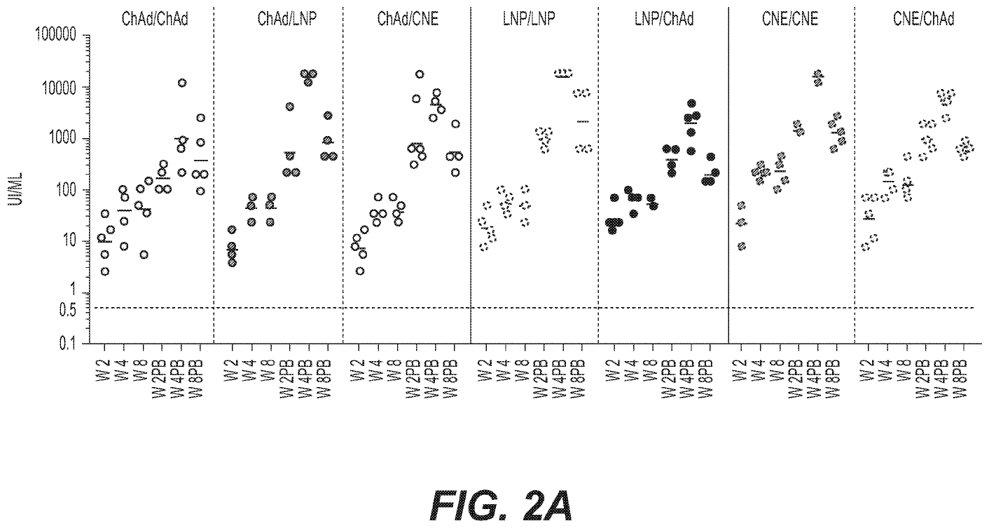

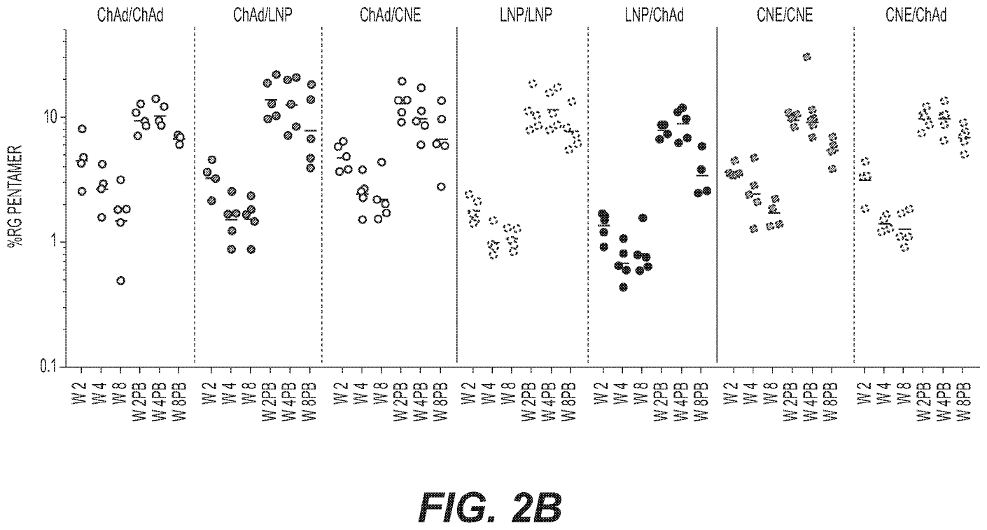

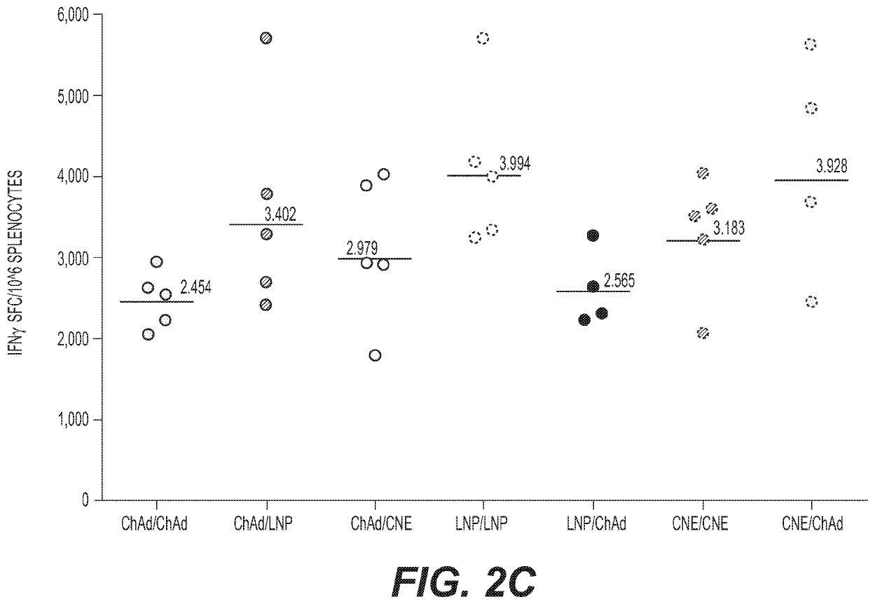

FIG. 2A shows the antibody immune response to the prime boost regimens shown in the table above. Serology at weeks 2, 4 and 8 demonstrated that a single intramuscular vaccination of adenovirus-RG or RNA-RG elicited virus neutralizing antibody titers in all mice well above the protective threshold of 0.5 IU/ml. Boosting further expanded these responses as much as about two logarithms in the weeks post boost (“wpb”). Heterologous adenoviral prime and RNA boost regimens were as efficient as homologous RNA prime boost in raising the magnitudes of the resulting titers. RNA appeared to be a more potent booster than adenovirus, based on the increase in titer post-boost.

Analysis of antigen specific T cells quantified from whole blood over time showed that prime/boost vaccinations with adenovirus-RG and RNA-RG elicited strong CD8+ T cell responses to the RG antigen, and that the heterologous adenovirus/RNA regimens were among the most potent vaccination regimens. FIG. 2B shows the effect of boosting on the CD8+ T cell response for each of the prime boost regimens. FIG. 2C shows the results of IFNγ ELISpot analysis of splenocytes at week 16. All regimens elicited strong, long lasting functional T cell responses to the RG antigen.

In summary, the data in Example 1 show that adenoviral and RNA vaccine platforms can be successfully combined in heterologous prime/boost regimens for eliciting and enhancing both humoral and cellular responses to an encoded model antigen. The responses were elicited with small microgram amounts of RNA.

### Example 2: HIV GAG as a Model Antigen for a Prime Boost Regimen

Adenoviral vectors encoding an HIV1 GAG antigen transgene were cloned and used to prepare adenoviral particles in chimpanzee adenovirus 155 (ChAd155). Self-amplifying RNA vectors encoding the HIV1 GAG antigen sequence were used to prepare in vitro transcribed capped RNA (SAM-HIV1).

Adenoviral vectors and RNAs were each characterized for in vitro potency and formulated for vaccine injection in mice. Adenoviral particles were formulated in Tris-NaCl. SAM-HIV1 GAG was formulated in lipid nanoparticles (LNP), using RV39 as the lipid.

Single Administration of HIV1 GAG

Six week old female BALB/c mice were allocated into groups of twenty and the adenoviruses or RNAs were administered intramuscularly according to the regimens shown in the table below. The animals were bled at weeks 2, 4, 6 and 8 for antibody analysis and T cell response. Five animals in each group were sacrificed at each of weeks 2, 4, 6 and 8 and the spleens were collected to determine antigen specific T cell responses.

| Group | Antigen | Vector | Formulation | Dose | |
|---|---|---|---|---|---|
| 1 | HIV1 GAG | Saline | Saline | 0 | |
| 2 | HIV1 GAG | Adenovirus | Tris-NaCl | 3 × 10⁶ vp | |
| 3 | HIV1 GAG | Adenovirus | Tris-NaCl | 10⁷ | vp |
| 4 | HIV1 GAG | Adenovirus | Tris-NaCl | 10⁸ | vp |
| 5 | HIV1 GAG | RNA | LNP | 0.15 | ug |
| 6 | HIV1 GAG | RNA | LNP | 1.5 | ug |

An analysis of HIV1-specific humoral and cellular immune responses was performed on samples taken during the eight weeks post-immunization. HIV1 specific total IgG titers were measured by ELISA.

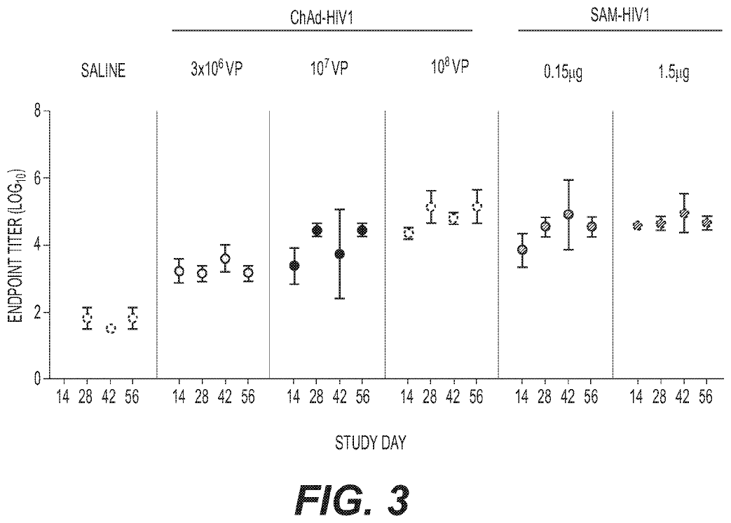

FIG. 3 shows the antibody immune response after one dose of either adenovirus or RNA encoding the HIV1 GAG antigen. Both vaccines induced high antibody titers at days 14-56, expressed as a logarithm of the measured titer, compared to a saline control. The adenoviral-HIV1 titers were dose dependent over the tested doses of 3×10⁶ vp, 107 vp and 10⁸ vp. RNA-HIV1 at both doses induced similar responses to those elicited by ChAd at the highest dose.

HIV1 antigen specific CD8+ T cells in whole blood were quantified using a conjugated pentamer consisting of an AMQMLKET immunodominant CD8+ T cell epitope that binds to T cell receptors specific for the major histocompatibility complex (MHC) class H-2. Whole blood was collected at weeks 2, 4, 6, and 8 and stained with the H-2d-restricted HIV1 GAG-specific CD8+ pentamer and fluorochrome labelled antibodies for T cell markers. Positive antigen specific CD8+ T cells were measured by flow cytometry.

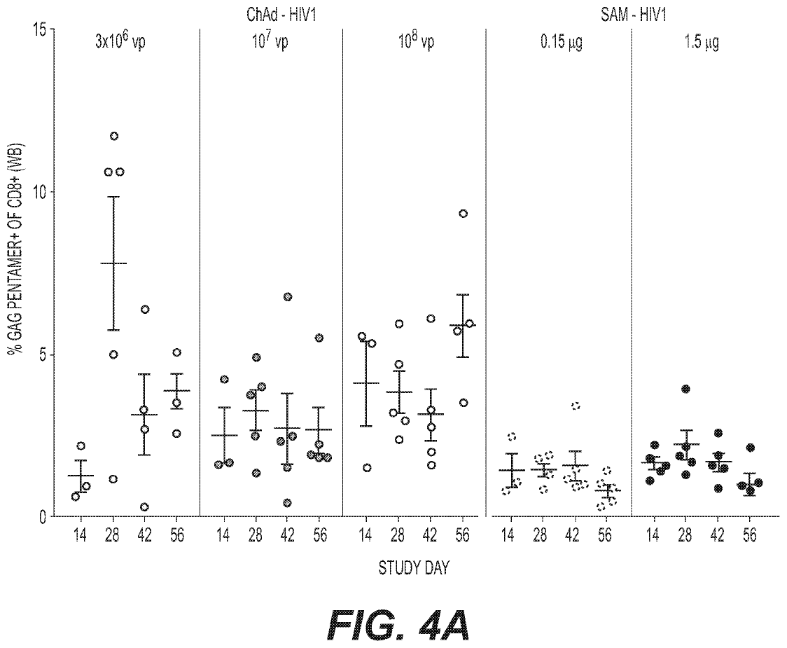

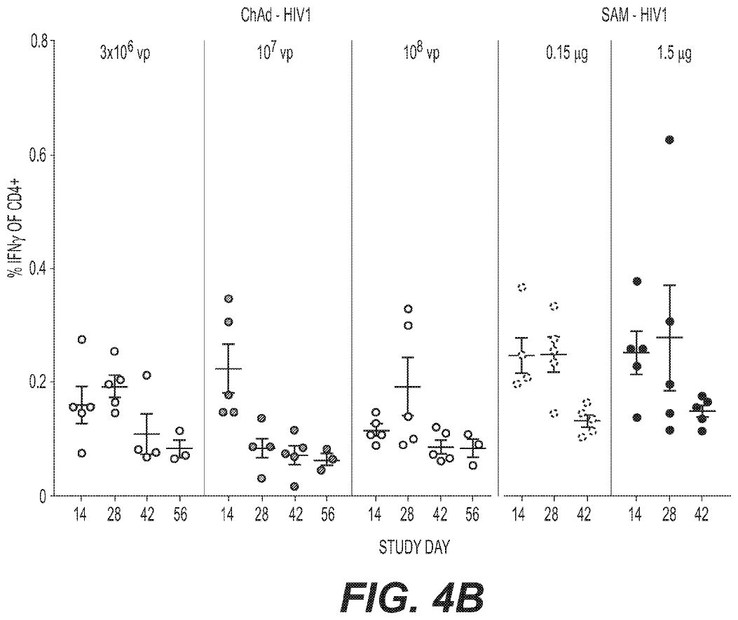

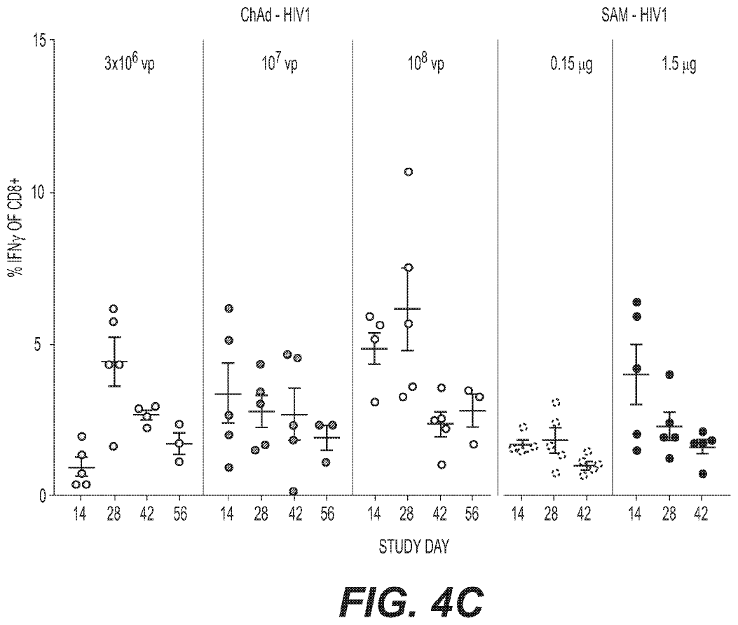

FIG. 4A shows the CD8+ T cell response after one dose of either adenovirus or RNA encoding the HIV1 antigen. Data are expressed as frequency of HIV1 GAG-specific (pentamer+) cells within the CD8+ T cell population. Vaccination with either adenovirus-HIV1 or RNA-HIV1 elicited strong CD8+ T cell responses, with the adenoviral construct eliciting more pentamer positive cells than the RNA construct.

Functional T cell responses of splenocytes were measured by intracellular cytokine staining (ICS) using antigen pools of overlapping 15mer peptides encompassing the HIV GAG protein sequence. ICS analysis of splenocytes showed that IFNγ responses in CD4+ T cells were detected, although at low frequencies (FIG. 4B). Both adenovirus-HIV1 and RNA-HIV1 vaccines elicited strong functional CD8+ T cell responses to the antigen (FIG. 4C). The higher doses of both the adenoviral and RNA constructs reached peak response earlier than the lower doses, with peak IFN-γ secretion observed at 2-4 weeks post vaccination by both the adenoviruses and RNAs.

Prime/Boost with HIV1-GAG

### Experiment 1

Based on the results of the single administration, the priming doses of 107 vp ChAd-HIV1 and 0.015 μg SAM/LNP-HIV1 were selected for priming in a prime/boost vaccination regimen as the lowest effective doses that were able to confer immunogenicity levels that were comparable between the adenovirus-HIV1 and the RNA-HIV1 vaccines after priming. Two RNA boosting doses were tested, as shown in the table below. The interval between prime and boost was eight weeks.

Female BALB/c mice six to eight weeks of age were allocated into groups of either ten or twenty and the ChAd or SAM vectors were administered intramuscularly in regimens shown in the table below. The animals in groups 1-3 were bled at 2, 4, 6 and 8 weeks after priming and monthly thereafter. All animals were bled at week 10 and monthly thereafter. A heterologous group primed with adenovirus-HIV1 and boosted with Modified Vaccinia Ankara (MVA) virus was added as a positive control. Serology for neutralizing antibodies and T cell assays were performed as with the single administration.

| | | Priming | | | Boosting | |
|---|---|---|---|---|---|---|
| | Priming | Dose | | Boosting | Dose | |
| Group | Vector | (week 0) | | Vector | (week 8) | |
| 1 | Saline | n/a | | Saline | n/a | |
| 2 | Adenovirus- | | | | | |
| | HIV1 | 10⁷ | vp | None | n/a | |
| 3 | SAM RNA- | 0.15 | ug | None | n/a | |
| | HIV1/LNP | | | | | |
| 4 | Adenovirus- | 10⁷ | vp | MVA-HIV1 | 10⁶ | vp |
| | HIV1 | | | | | |
| 5 | Adenovirus- | 10⁷ | vp | SAM RNA - | 0.15 | ug |
| | HIV1 | | | HIV1/LNP | | |
| 6 | Adenovirus- | 10⁷ | vp | SAM RNA - | 1.5 | ug |
| | HIV1 | | | HIV1/LNP | | |
| 7 | Adenovirus- | 10⁷ | vp | Adenovirus- | 10⁷ | vp |
| | HIV1 | | | HIV1 | | |
| 8 | SAM RNA - | 0.15 | ug | Adenovirus- | 10⁷ | vp |
| | HIV1/LNP | | | HIV1 | | |
| 9 | SAM RNA - | 0.15 | ug | SAM RNA - | 0.15 | ug |
| | HIV1/LNP | | | HIV1/LNP | | |

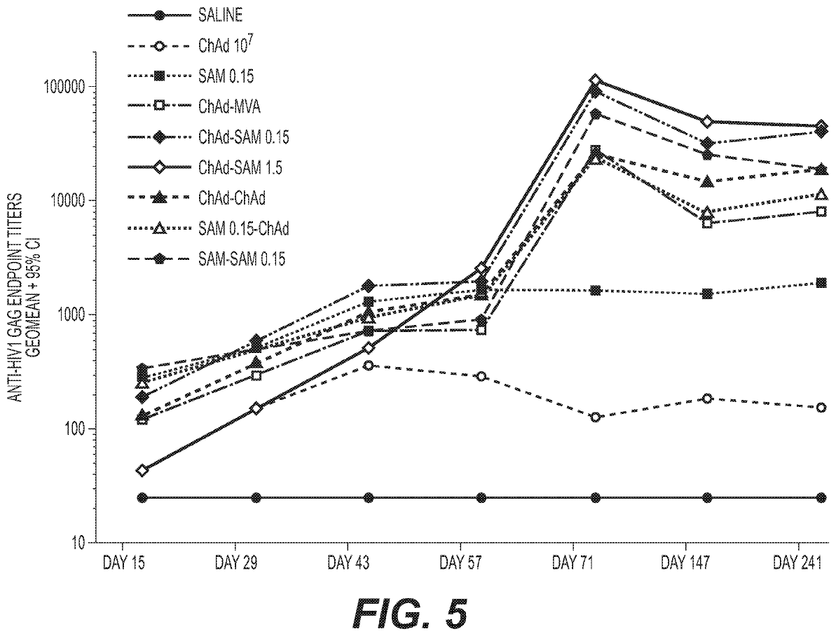

FIG. 5 shows the antibody immune responses measured at days 15, 29, 43, 57 (day of boost) after prime and days 71, 147 and 241 after the prime boost regimens shown in the table above. HIV1 GAG specific IgG titer, determined by ELISA analysis, showed that a single intramuscular vaccination of adenovirus-HIV1 or RNA-HIV1 elicited antigen-specific IgG titers in all of the mice and the responses were boosted by the second immunization in all groups. Heterologous adenovirus-HIV1 prime and RNA-HIV1 boost regimens showed a trend of producing higher IgG titers than either homologous adenovirus-HIV1 prime or RNA HIV1 boost regimens and also trended higher than heterologous adenovirus-HIV1 prime with MVA boost. All antibody immune responses were sustained for at least 241 days.

As shown in FIG. 5, a boosting effect was observed in all boosted groups. The strongest antibody response was observed with adenovirus as the priming agent and SAM as the boosting agent, exceeding even the response elicited by an adenoviral prime and an MVA boost, which has been described in the art as an effective vaccination method.

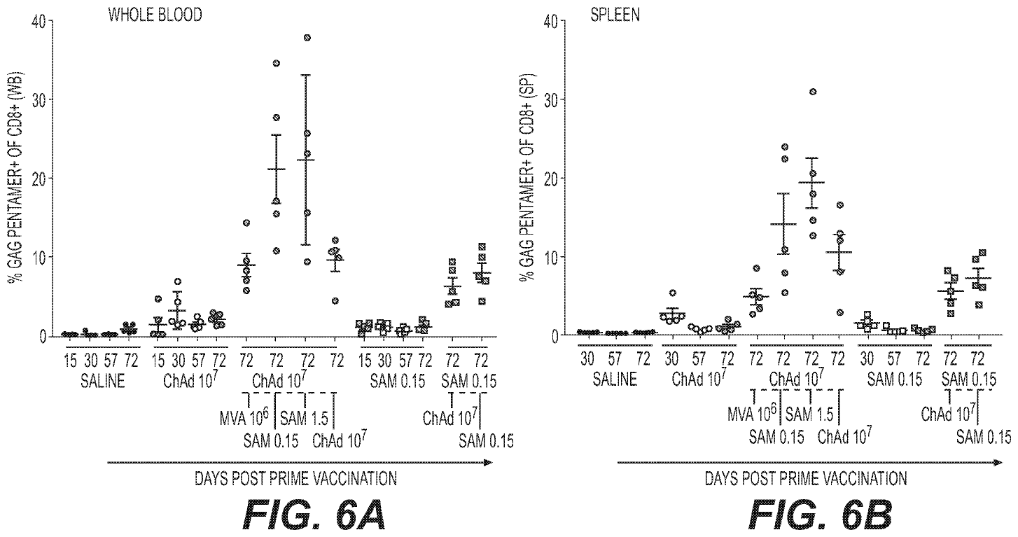

The CD8+ T cell response was quantified by an HIV1 GAG-specific binding assay. HIV1 GAG specific CD8+ T cells were quantified by staining with an H2 Kd restricted pentamer of the amino acid sequence AMQMLKET. FIG. 6 shows the results of GAG-specific CD8+ T cell response by pentamer staining performed with whole blood (FIG. 6A) and splenocytes (FIG. 6B). FIG. 6A shows that priming with adenovirus-HIV1 and boosting with either MVA-HIV1, RNA-HIV1 or adenovirus-HIV1 elicits a strong CD8+ T cell response in the peripheral blood circulation. The response to an adenovirus/RNA heterologous prime boost regimen was superior to that of an adenovirus/MVA regimen. FIG. 6B shows a similar response from T cells in the spleen.

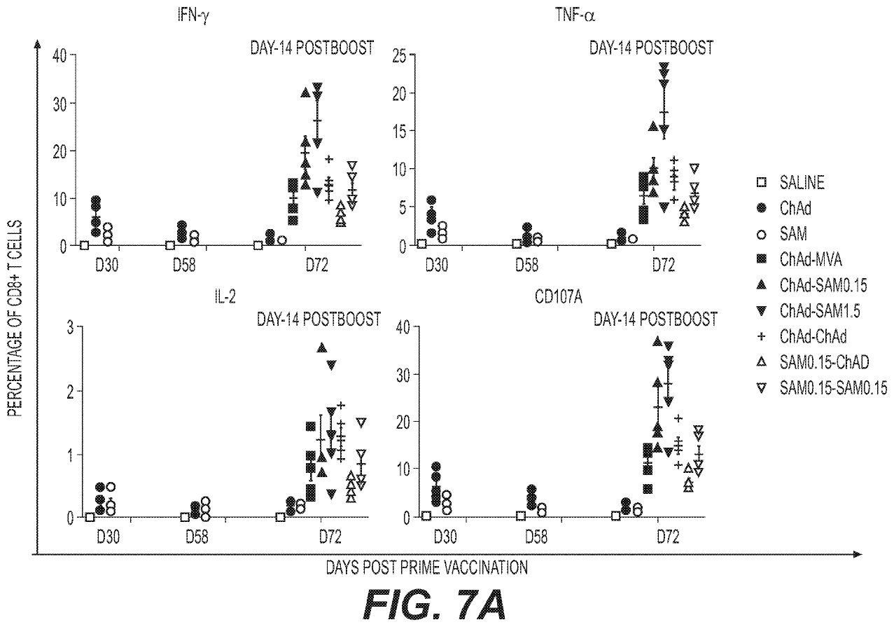

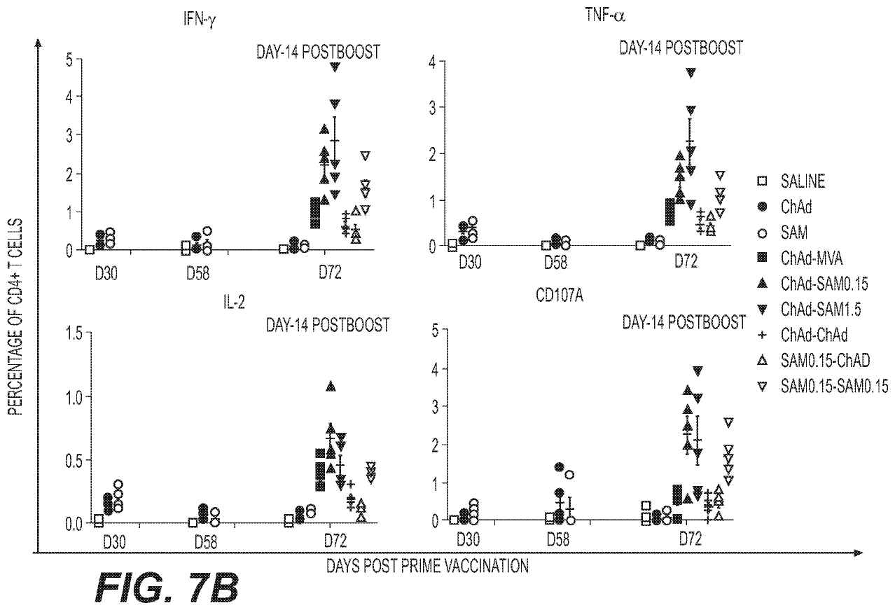

FIG. 7 shows the results of intracellular cytokine staining (ICS) for IFN-γ, TNFα, interleukin 2 (IL-2) and for CD107a, which is a marker for natural killer cell activity. ICS analysis of splenocytes confirmed that all the regimens shown in the table above elicited strong, functional T cell responses to the HIV1 GAG antigen, with heterologous adeno/RNA combinations showing both the highest CD8+ T cell response (FIG. 7A), and CD4+ T cell response (FIG. 7B). Adenovirus/adenovirus, adenovirus/MVA, and RNA/RNA induced overall equivalent levels of CD8+ and CD4+ T cell responses, with some variation from one cytokine to another (FIGS. 7A and B).

At six months post boost, the GAG-pentamer specific CD8+ T cells are mainly central memory and effector memory T cells, rather than effector T cells. Animals primed with adenovirus-HIV1-GAG and boosted with RNA-HIV1-GAG showed a greater increase in both CD4+/IFN [symbol] + T cells and CD8+/IFN [symbol] + T cells at six months post boost than the other prime boost regimens.

Consistent with the data shown in Example 1, the data generated with a second model antigen shows that adenovirus and RNA vaccine platforms can be successfully combined in heterologous prime/boost regimens that elicit and enhance both humoral and cellular responses to an encoded antigen. The heterologous adenovirus prime/RNA boost combination that enhanced the HIV1-specific immune response was somewhat more efficient than the adenovirus prime/MVA boost combination. Again, the responses were elicited with small microgram amounts of RNA.

### Experiment 2

A second experiment was performed to determine the kinetics of the T cell responses to heterologous priming and boosting with simian adenovirus and self-amplifying RNA. Balb/c mice were allocated into eight groups of either twenty (groups 3-8)) or thirty (groups 1 and 2) and given an intramuscular priming dose of 1×10⁷ vp ChAd 155-HIV1 GAG and boosted intramuscularly on day 57 with adenovirus, SAM RNA or MVA, as shown in the table below. Whole blood was collected on days 14, 28, 42, 56, 64, 72 and 100 for analysis of the T cells in the circulating bloodstream. The mice were sacrificed and their spleens were collected on days 28, 56, 64, 72 and 100 for in vitro stimulation with an HIV GAG peptide pool followed by T cell intracellular cytokine staining for IFNgamma, TNFalpha, IL2 and CD107a to determine T cell functionality.

| Group | Prime Vaccine | Prime Dose | Boost Vaccine | Boost Dose | |
|---|---|---|---|---|---|
| 1 | Saline | 0 | Saline | 0 | |
| 2 | Adenovirus | 1 × 10⁷ vp | None | N/A | |
| 3 | Adenovirus | 1 × 10⁷ vp | Adenovirus | 1 × 10⁷ | vp |
| 4 | Adenovirus | 1 × 10⁷ vp | SAM RNA | 0.015 | ug |
| 5 | Adenovirus | 1 × 10⁷ vp | SAM RNA | 0.15 | ug |
| 6 | Adenovirus | 1 × 10⁷ vp | SAM RNA | 1.5 | ug |
| 7 | Adenovirus | 1 × 10⁷ vp | MVA | 1 × 10⁶ | pfu |
| 8 | Adenovirus | 1 × 10⁷ vp | MVA | 1 × 10⁷ | pfu |

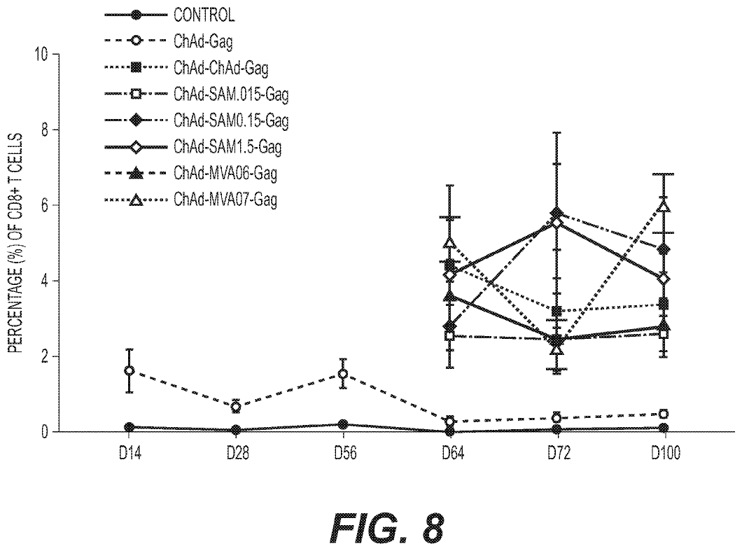

FIG. 8 shows the CD8+ T cell response as quantified with a flow cytometry based staining assay after binding to a pentamer specific for HIV1 GAG and expressed as the percentage of total CD8+ T cells. HIV1 GAG specific CD8+ T cells in whole blood were quantified by staining with an H2 Kd restricted pentamer of the amino acid sequence AMQMLKET. Priming with adenovirus HIV1 GAG and boosting with either adenovirus, SAM or MVA elicited a strong CD8+ T cell response in the peripheral blood circulation. By one week post boost all boosting regimens were effective, with a similar percentage of pentamer-positive cells in all groups. At two weeks post-boost, the strongest response was observed with the SAM boost, which was more effective than MVA. The response to the heterologous adeno/SAM prime boost peaked at two weeks post boost (approximately day 72) and was superior to a homologous adeno/adeno prime boost.

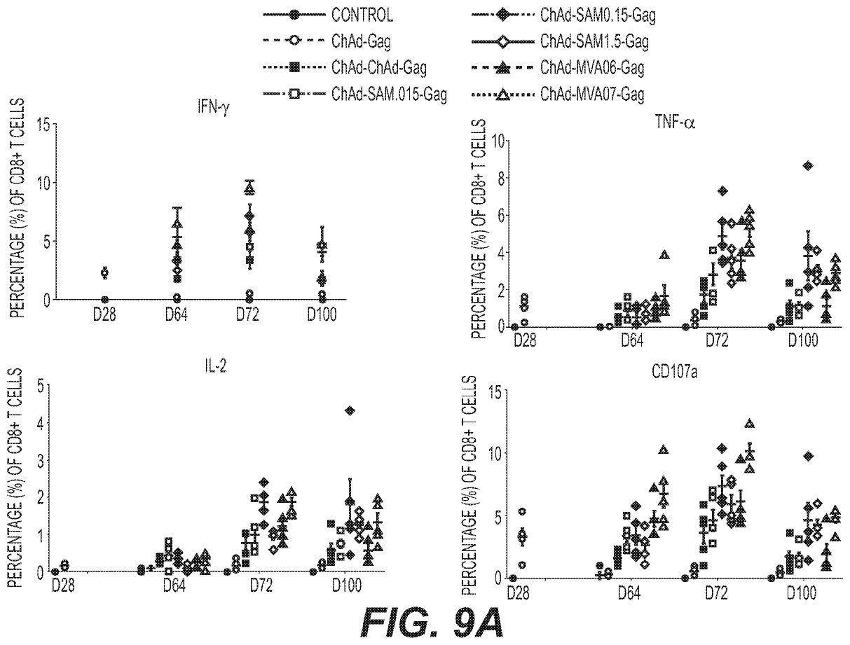

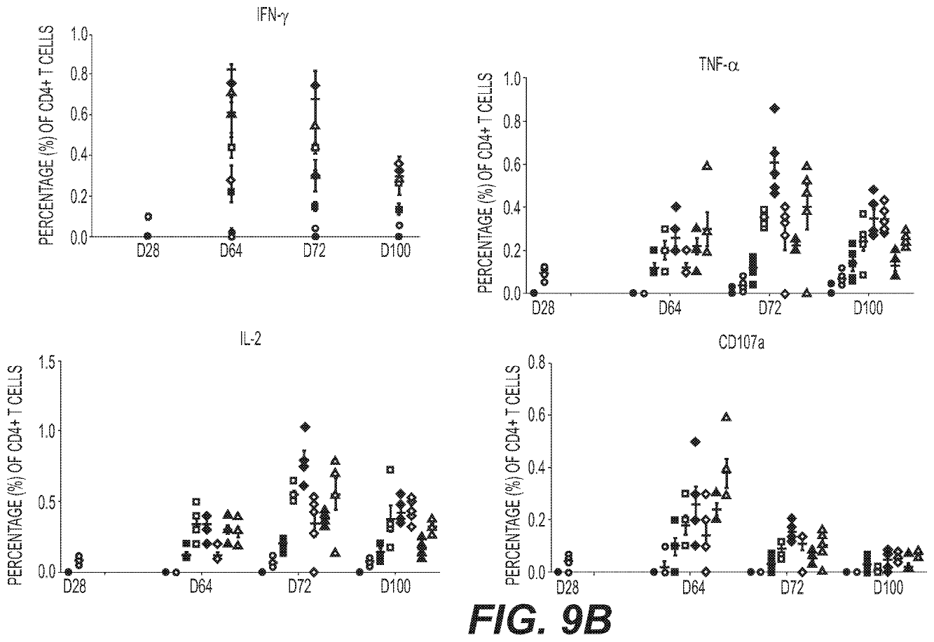

Functional T cell responses of the splenocytes were then measured by intracellular cytokine staining (ICS) using antigen pools of overlapping 15mer peptides encompassing the HIV GAG protein sequence, as in Experiment 1. All of the heterologous prime boosts elicited polyfunctional responses from splenic CD8+ T cells (FIG. 9A). All of the booster vaccines at every dose tested predominantly induced GAG-specific CD107a+/IFNgamma+ and CD107+/IFNgamma+ and TNFalpha+polyfunctional cytotoxic CD8+ T cells. At day 72, all of the booster vaccines and doses induced robust expression of CD107a, IFNgamma and TNFalpha. The fraction of the total CD8+ T cells that express all four cytokines was increased at day 100 compared to days 64 and 72 by all booster vaccines at all doses.

Both SAM and MVA boosted adenoviral primed CD8+ T cell responses. The booster responses were dose dependent between 0.015 and 0.15 μg SAM and between 1×10⁶ and 1×10⁷ vp MVA, with peak responses occurring approximately two weeks post boost. FIG. 9A shows the results of intracellular cytokine staining of INFgamma, TNFalpha, IL-2 and CD107a in splenic CD8+ T cells. As observed in Experiment 1, all prime boost regimens elicited strong functional CD8+ T cell responses. Peak CD8+ IFNgamma, CD107a and TNFalpha responses were observed two weeks post boost (approximately day 72). All of the booster doses predominantly induced Gag-specific CD107a+/IFNgamma+ and CD107a+/IFNgamma+/TNFalpha+ polyfunctional cytotoxic CD8+ T cells. The polyfunctionality of the CD8+ T-cells was observed to increase between week 1 and week 2 post boost, when a higher proportion of quadruple- and triple-cytokine positive cells appeared.

Both SAM and MVA also boosted adenoviral primed CD4+ T cell responses, although the responses were overall lower than those demonstrated by CD8+ T cells. FIG. 9B shows the results of intracellular cytokine staining of IFNgamma, TNFalpha, IL-2 and CD107a in splenic CD4+ T cells. All of the booster vaccines at each of the doses predominantly induced IFNgamma+/TNFalpha+/IL-2+, suggestive of Th1/Th0 polyfunctional CD4+ T cells. Diversity of the response increased after day 64, with a greater variety of cytokines expressed.

The kinetics and dose-response of the CD4+ T cells were similar to that of the CD8+ T cells, with the peak of the response observed at one week post-boost for CD107a and IFNgamma and two weeks post boost for IL-2 and TNFalpha. The potency of the SAM boost and the MVA boost were similar. The polyfunctionality of CD4+ T cells increased from week 1 to weeks 2-6 post boost.

In summary, both Experiment 1 and Experiment 2 demonstrate that heterologous prime-boost vaccination with a simian adenovirus encoding an HIV-GAG antigen prime followed by a self-amplifying RNA encoding an HIV-GAG antigen boost induced robust CD4+ and CD8+ T-cell responses. Boosting with either SAM or MVA induced stronger responses than homologous boosting with adenovirus. The polyfunctionality of CD8+ T cells induced by all booster doses increased from about day 64 to about day 100, i.e., one week post boost to six weeks post-boost. Responses were predominantly cytotoxic (CD107a) and were also positive for IFN-γ+/TNF-α+.

### Example 3: HSV as a Model Antigen for a Prime Boost Regimen

Simian adenoviral vectors encoding a herpes simplex virus (HSV) Gly VI antigen transgene (PCT/EP2018/076925) were cloned and used to prepare adenoviral particles in ChAd155 (ChAd-HSV). The HSV Gly VI antigen transgene encodes a polyprotein formed by selected immunodominant sequences from the five HSV antigens UL-47, UL-49, UL-39, ICP0 and ICP4. A self-amplifying RNA vector encoding the same antigen sequence was cloned and used to prepare in vitro transcribed capped RNA (SAM-HSV).

Adenoviral vectors and self-amplifying RNA encoding HSV Gly VI were each characterized for in vitro potency and formulated for vaccine injection in mice. Adenoviral particles were formulated in Tris-NaCl. SAM-HSV was formulated as lipid nanoparticles (LNP) with RV39 as the lipid.

Single Administration of HSV Antigen

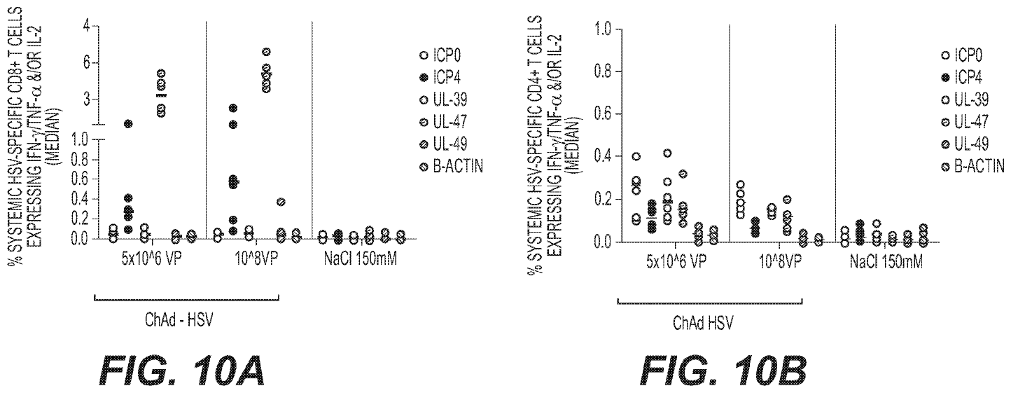

Naïve CB6F1 inbred mice were administered either saline, 5×10⁶ vp or 10⁸ vp adenovirus-HSV intramuscularly in groups of six. Twenty days after this priming immunization, six mice in each group were sacrificed for T cell analysis. Splenocytes were harvested and stimulated ex-vivo for six hours with pools of 15mer peptides covering the amino acid sequences of the five HSV antigens (ICP0, ICP4, UL-39, UL-47, UL-49). A pool of 15mer peptides covering the amino acid sequence of beta-actin served as a negative control. The frequencies of HSV-specific CD8+ (FIG. 10A) and CD4+ (FIG. 10B) T cells secreting any or all IFN-γ, IL-2 or TNF-α were measured by intracellular cell staining. The cut-off value for identifying specific CD4+/CD8+ T cell responses in vaccine-immunized mice corresponds to the 95^{th} percentile of the T cell responses obtained in the saline group.

FIG. 10A shows that the mice displayed polyfunctional HSV-specific CD8+ T cell responses after immunization with ChAd-HSV. Compared to saline treated mice, immunized mice elicited polyfunctional HSV-specific CD8+ T cell responses towards certain of the transgenic HSV antigens, with the dominant CD8+ response directed to the UL-47 antigen. HSV-specific CD8+ T cell responses against the ICP0, UL-39 and UL-49 antigens were not detected after a single dose of adenovirus-HSV. Mice administered 5×10⁶ vp had a weaker CD8+ T cell response than those administered 10⁸ vp (FIG. 10A), suggesting that the magnitude of CD8+ T cell responses are both dose and antigen dependent.

FIG. 10B shows that the mice also displayed polyfunctional HSV-specific CD4+ T cell responses after immunization with adenovirus-HSV. The dominant CD4+ T cell responses were directed to the ICP0 and UL-39 antigens, with fewer mice displaying CD4+ T cell responses against ICP4 and UL-47.

In a related study, naïve inbred CB6F1 mice were immunized intramuscularly with either saline or 10⁸ vp adenovirus-HSV. At day 20 post immunization, splenocytes were isolated and stimulated ex-vivo for six hours with a pool of 15mer peptides covering the amino acid sequence of the UL-47 antigen. The poly-functional profiles of UL-47-specific CD8+ T cells were evaluated by measuring IFN-γ, IL-2 and TNF-α cytokine production.

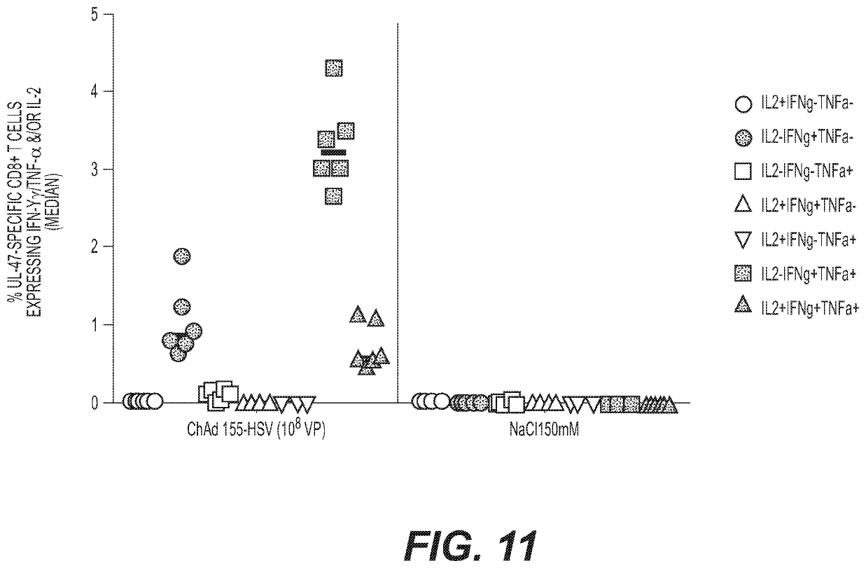

As shown in FIG. 11, the most dominant UL-47-specific CD8+ T cell response to adenovirus-HSV was to secrete IFN-γ and TNF-α but not IL-2. Cytokine responses to the UL-47 antigen also included cohorts of CD8+ T cells that secreted (a) IFN-γ but not TNF-α or IL-2 and (b) IFN-γ, TNF-α and IL-2.

Prime/Boost with HSV

Naïve CB6F1 inbred mice were immunized intramuscularly in groups of five with either 5×10⁶ vp or 10⁸ vp ChAd-HSV. At day 57, the mice immunized with the lower dose were heterologously immunized intramuscularly with 1 μg of LNP-formulated SAM-HSV. A third group of mice was immunized at days 0 and 57 with saline as a negative control. Mice were sacrificed for T cell analysis 25 days after the second immunization, i.e., 82 days post priming. Splenocytes were harvested and stimulated ex-vivo for six hours with pools of 15mer peptides covering the amino acid sequences of the five HSV antigens (ICP0, ICP4, UL-39, UL-47, UL-49). A pool of 15mer peptides covering the amino acid sequence of beta-actin served as a negative control.

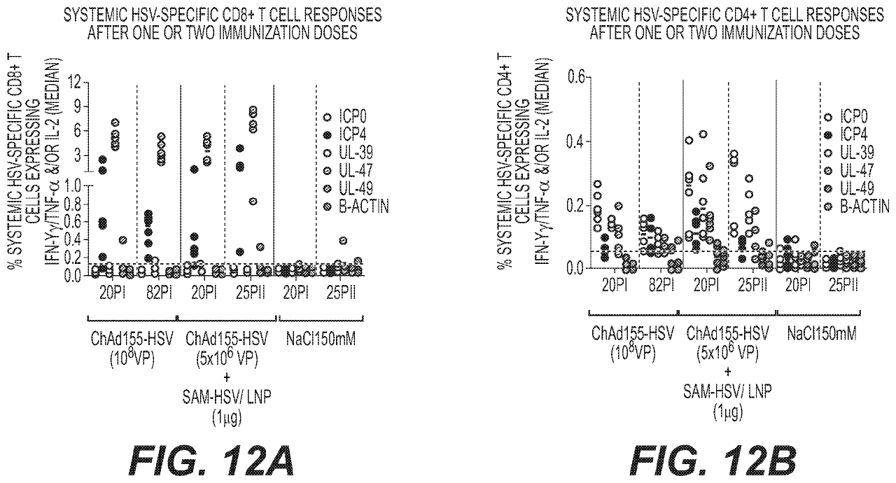

The frequencies of HSV-specific CD8+ (FIG. 12A) and CD4+ (FIG. 12B) T cells secreting IFN-γ, IL-2 or TNF-α were measured by intracellular staining. The cut-off value for identifying specific CD4+/CD8+ T cell responses in vaccine-immunized mice corresponds to the 95^{th} percentile of T cell responses obtained in the saline group.

Consistent with the data shown in FIG. 10, by 20 days after priming a trend for dominant CD8+ T cell responses towards UL47 and ICP4 antigens was observed. As shown in FIG. 12A, at day 20 after the priming immunization (20 PI) with adenovirus-HSV, CD8+ T cells produced IFN-γ, TNF-α and/or IL-2 in response to UL-47 and ICP4 and to a lesser degree in response to the ICP0 and UL-49 antigens. This response was also observed at day 82 post-prime (82 PI).

Also shown in FIG. 12A is the CD8+ T cell response after priming with 10⁸ vp of adenovirus-HSV and boosting with RNA-HSV (heterologous prime/boost). At day 20 after the priming immunization (20 PI), CD8+ T cells produced IFN-γ, TNF-α and IL-2 in response to UL-47 and ICP4. At day 25 following the booster immunization (25 PII), i.e., 82 days post-prime, the intensity of the CD8+ T cell responses to UL-47 and ICP4 was increased compared to the responses in the group immunized once with adenovirus-HSV. Mice primed and boosted with saline did not secrete cytokines from splenic CD8+ T cells. Thus, RNA-HSV was able to boost the pre-existing CD8+ T cell responses induced by adenovirus-HSV (FIG. 12A).

The CD4+ T cell response observed as a result of the prime boost regimen (FIG. 12B) was also consistent with that observed after one dose (FIG. 10B). As shown in FIG. 12B, at day 20 after the priming immunization (20 PI) with 10⁸ vp of adenovirus-HSV, CD4+ T cells produced IFN-γ, TNF-α and/or IL-2 in response to the HSV transgene. This response was also observed 25 days after the booster immunization, i.e., day 82 post-priming (82 PI).

Also shown in FIG. 12B is the CD4+ T cell response after priming with 10⁸ vp of adenovirus-HSV and boosting with RNA-HSV (heterologous prime/boost). At day 20 after the first immunization (20 PI) with adenovirus-HSV, CD4+ T cells produced IFN-γ, TNF-α and/or IL-2 in response to IPC0 and UL-39. At day 25 following a booster dose (25PII) of RNA-HSV, i.e., 82 days post-prime (82 PI), the intensity of the CD4+ T cell responses to UL-47 and ICP4 was increased compared to the responses in the group immunized once with adenovirus-HSV. Mice primed and boosted with saline did not secrete cytokines from splenic CD4+ T cells. Thus, RNA-HSV was able to boost the pre-existing CD4+ T cell responses induced by ChAd-HSV (FIG. 12B).

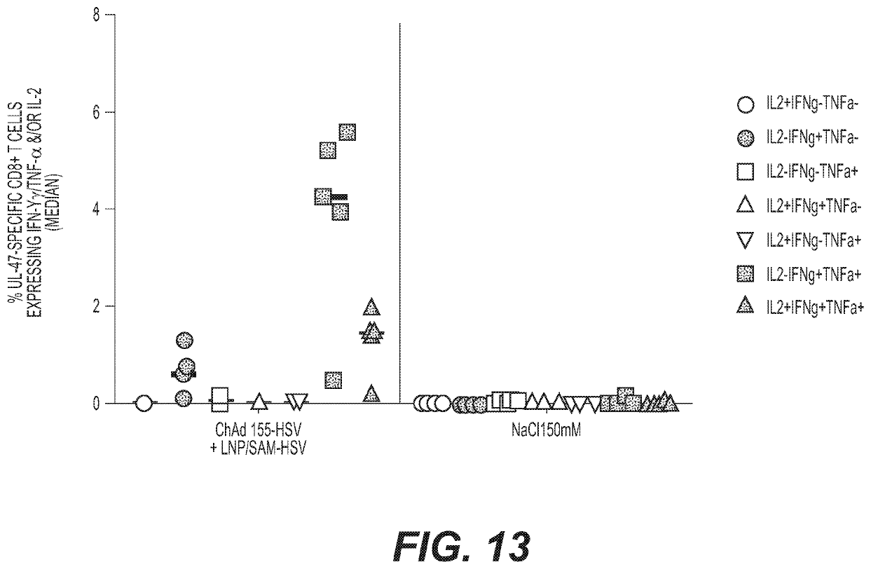

The polyfunctional profile of UL-47-specific CD8+ T cell response after adenovirus-HSV/RNA-HSV heterologous prime/boost immunization was examined and the results shown in FIG. 13. Naïve inbred CB6F1 mice were immunized intramuscularly in groups of five mice each with 5×10⁶ vp adenovirus-HSV and boosted on day 57 with 1 μg LNP-formulated RNA-HSV. At day 25 post boost, splenocytes were harvested and stimulated ex-vivo for six hours with pools of 15mer peptides covering the amino acid sequences of the UL-47 antigen. The polyfunctional profile of HSV-specific CD8+ T cells elicited in response to the UL-47 antigen was determined by measuring IFN-γ, IL-2 and TNF-α production. The poly-functional cytokine level of release from UL-47-specific CD8+ T cells was similar between the first and second immunization doses. These results suggested that LNP-formulated RNA-HSV did not modify the antigenic and poly-functional profiles of CD8+ T cell responses induced by adenovirus-HSV.

Consistent with the data shown in Examples 1 and 2, the data generated with a third model antigen shows that adenovirus and self-amplifying RNA vaccine platforms can be successfully combined in heterologous prime boost regimens that elicit and enhance cellular immune responses to an encoded antigen. These responses were elicited with small microgram amounts of RNA.

1. A method of inducing an immune response to an infectious disease in a mammal comprising
a. administering a priming vaccine comprising an immunologically effective amount of one or more antigens encoded by either an adenoviral vector or an RNA molecule and
b. administering a booster vaccine comprising an immunologically effective amount of one or more antigens encoded by either an adenoviral vector or an RNA molecule,
wherein if the priming vaccine is encoded by an adenoviral vector the booster vaccine is encoded by an RNA molecule, and if the priming vaccine is encoded by an RNA molecule the booster vaccine is encoded by an adenoviral vector.

2. The method of claim 1 wherein the priming vaccine comprises an immunologically effective amount of one or more antigens encoded by an adenoviral vector and the boosting vaccine comprises an immunologically effective amount of one or more antigens encoded by an RIA molecule.

3. The method of claim 1 wherein the priming vaccine comprises an immunologically effective amount of one or more antigens encoded by an RNA molecule and the boosting vaccine comprises an immunologically effective amount of one or more antigens encoded by an adenoviral vector.

4. The method of claim 1 wherein the one or more antigens are from the same pathogenic organism.

5. The method of claim 4 wherein the one or more antigens are the same in the priming vaccine and the boosting vaccine.

6. The method of claim 4 wherein at least one of the epitopes of the one or more antigens are different in the priming and the boosting vaccine.

7. The method of claim 1 wherein the adenoviral vector is a simian adenoviral vector.

8. The method of claim 7 wherein the simian adenoviral vector is selected from a chimpanzee, bonobo, rhesus macaque, orangutan and gorilla vector.

9. The method of claim 8 wherein the simian adenoviral vector is a chimpanzee vector.

10. The method of claim 9 wherein the chimpanzee vector is selected from AdY25, ChAd3, ChAd15, ChAd19, ChAd25.2, ChAd26, ChAd27, ChAd29, ChAd30, ChAd31, ChAd32, ChAd33, ChAd34, ChAd35, ChAd37, ChAd38, ChAd39, ChAd40, ChAd63, ChAd83, ChAd155, ChAd157, ChAdOx1, ChAdOx2, SadV41, sAd4287, sAd4310A, sAd4312, SAdV31 and SAdV-A1337.

11. The method of claim 1 wherein the RNA molecule is a messenger RNA (mRNA) molecule.

12. The method of claim 11 wherein the mRNA molecule is a self-amplifying RNA vector.

13. The method of claim 1 wherein the antigen is encoded in an expression cassette comprising a transgene and regulatory elements necessary for the translation, transcription and/or expression of the transgene in a host cell.

14. The method of claim 13 wherein the antigen is a polypeptide antigen.

15. The method of claim 1, wherein the RNA molecule is delivered as a cationic nanoemulsion (CNE) or a lipid nanoparticle (LNP).

16. The method of claim 15, wherein the LNP comprises a cationic lipid selected from the group consisting of:

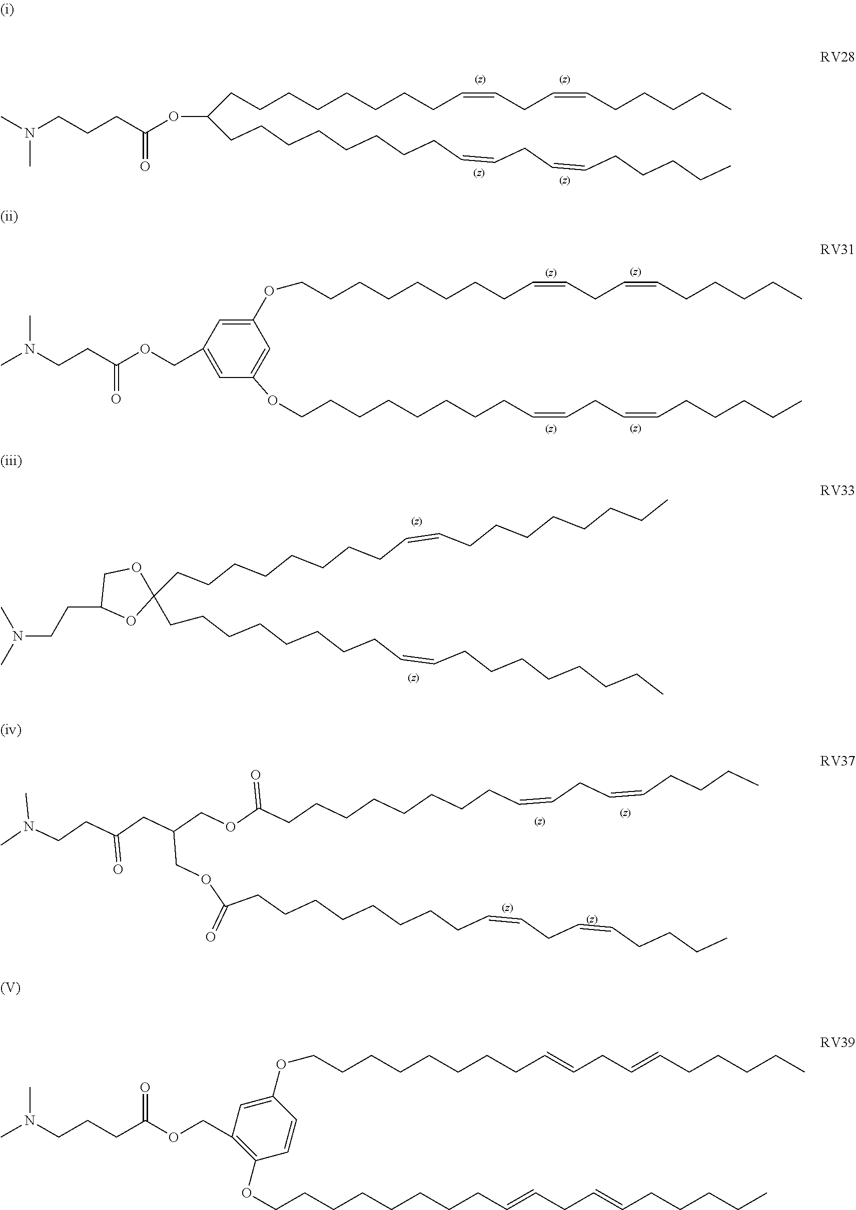

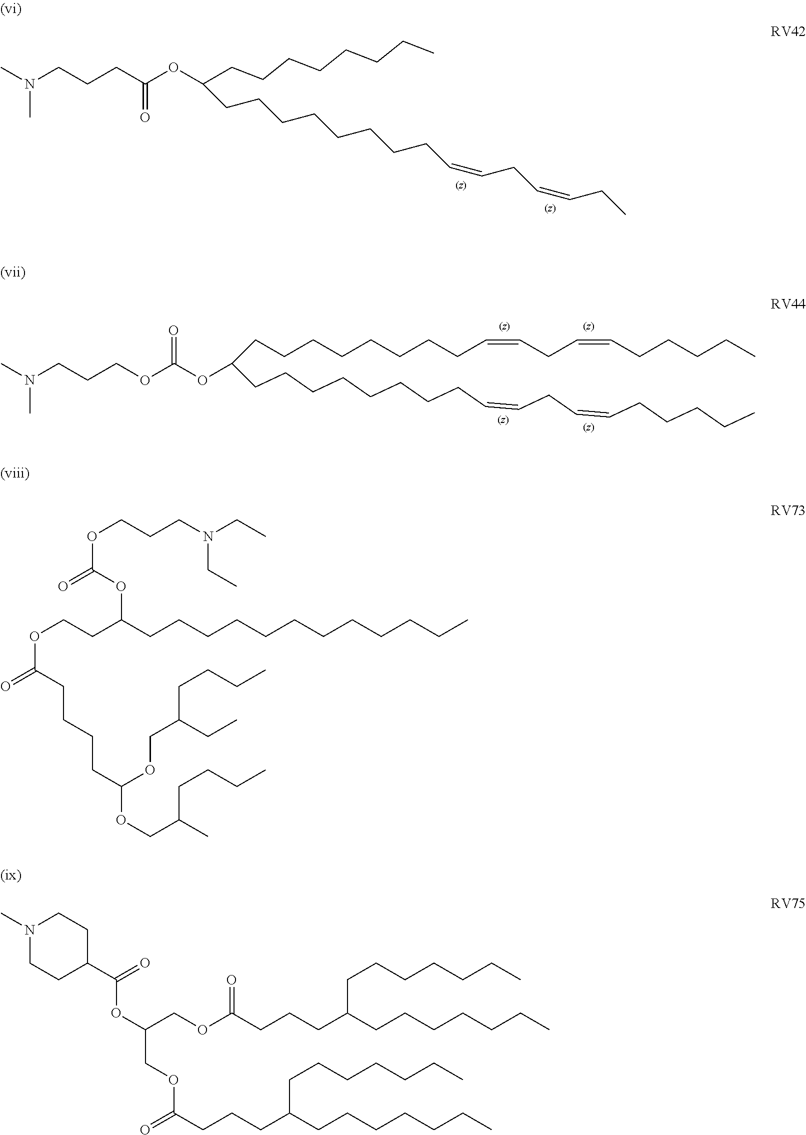

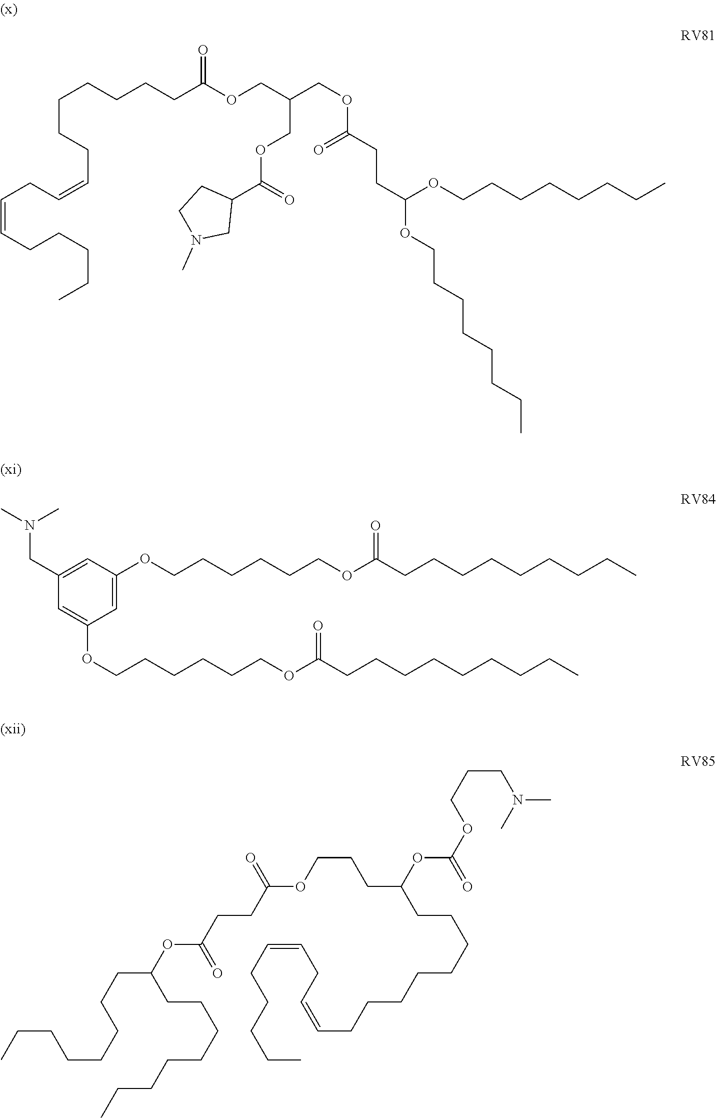

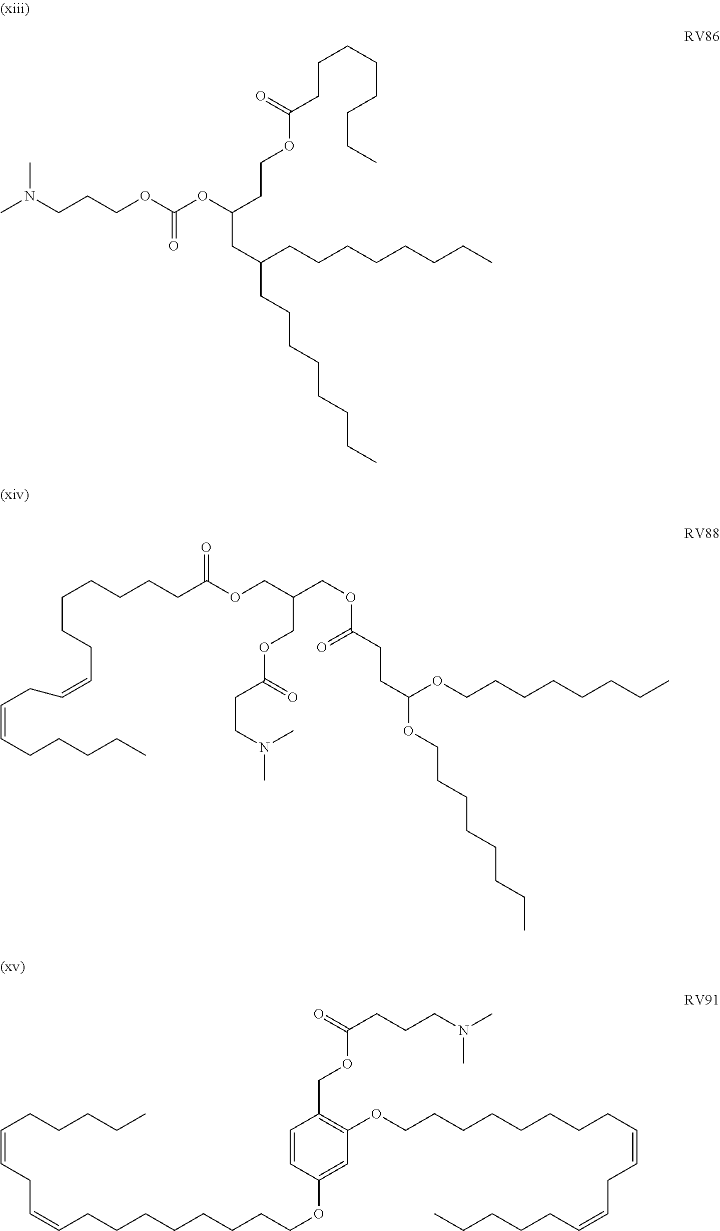 [SMILES: CCCCCCCCC(=O)OCCC(CC(CCCCCCCC)CCCCCCCC)OC(=O)OCCCN(C)C ; CCCCC/C=C\C/C=C\CCCCCCCC(=O)OCC(COC(=O)CCC(OCCCCCCCC)OCCCCCCCC)COC(=O)CCN(C)C ; CCCCC/C=C\C/C=C\CCCCCCCCOc1ccc(COC(=O)CCCN(C)C)c(OCCCCCCCC/C=C\C/C=C\CCCCC)c1]

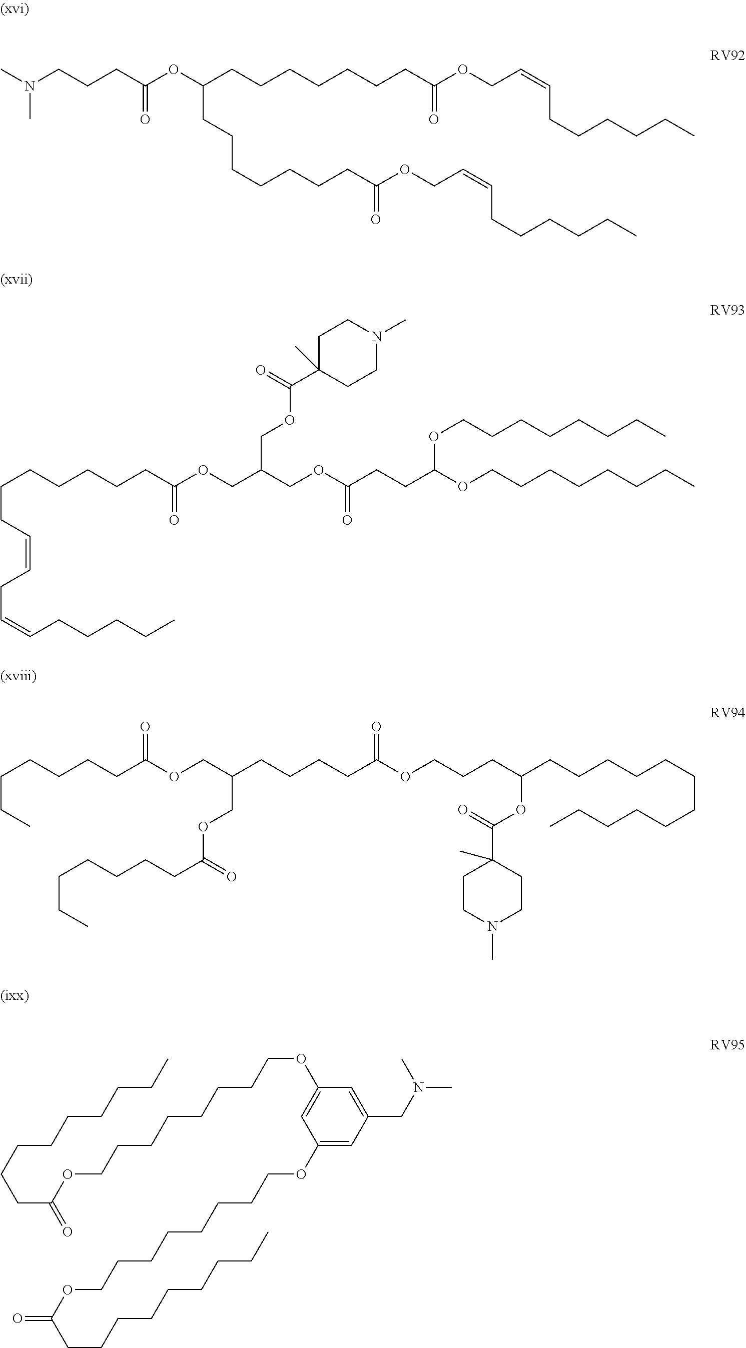 [SMILES: CCCCCC/C=C\COC(=O)CCCCCCCC(CCCCCCCC(=O)OC/C=C\CCCCCC)OC(=O)CCCN(C)C ; CCCCC/C=C\C/C=C\CCCCCCCC(=O)OCC(COC(=O)CCC(OCCCCCCCC)OCCCCCCCC)COC(=O)C1(C)CCN(C)CC1 ; CCCCCCCCCCCCC(CCCOC(=O)CCCCC(COC(=O)CCCCCCC)COC(=O)CCCCCCC)OC(=O)C1(C)CCN(C)CC1 ; CCCCCCCCCC(=O)OCCCCCCCCOc1cc(CN(C)C)cc(OCCCCCCCCOC(=O)CCCCCCCCC)c1]

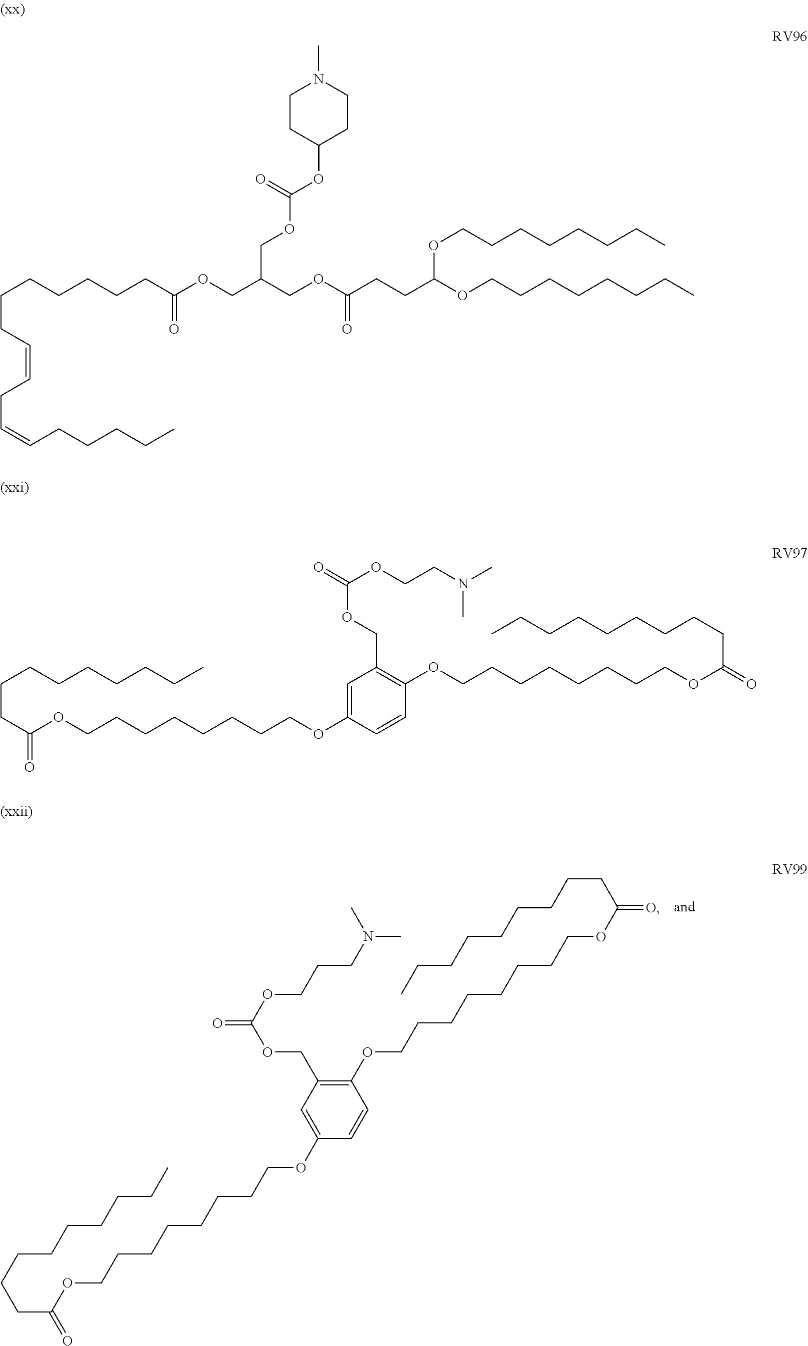 [SMILES: CCCCC/C=C\C/C=C\CCCCCCCC(=O)OCC(COC(=O)CCC(OCCCCCCCC)OCCCCCCCC)COC(=O)OC1CCN(C)CC1 ; CCCCCCCCCC(=O)OCCCCCCCCOc1ccc(OCCCCCCCCOC(=O)CCCCCCCCC)c(COC(=O)OCCN(C)C)c1 ; CCCCCCCCCC(=O)OCCCCCCCCOc1ccc(OCCCCCCCCOC(=O)CCCCCCCCC)c(COC(=O)OCCCN(C)C)c1]

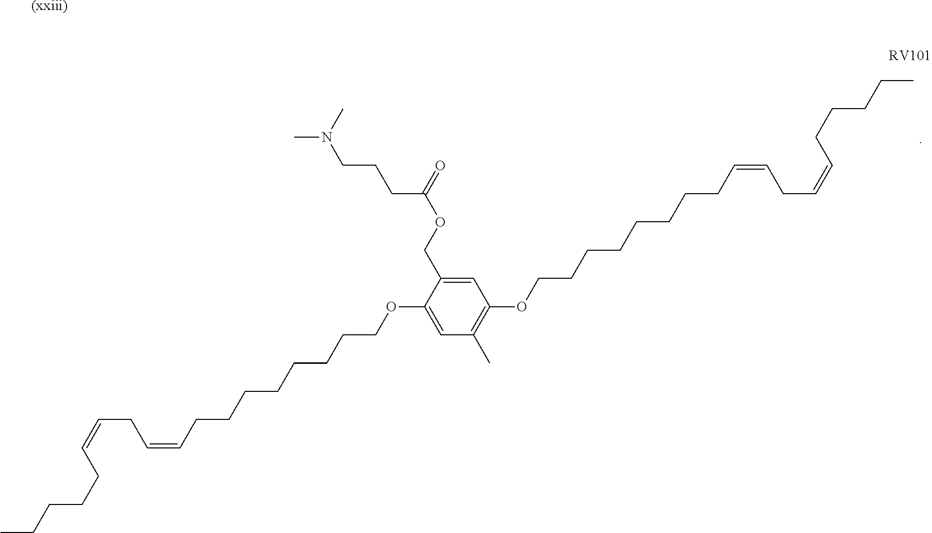 [SMILES: "(xxiii)" ; CCCCC/C=C\C/C=C\CCCCCCCCOc1cc(COC(=O)CCCN(C)C)c(OCCCCCCCC/C=C\C/C=C\CCCCC)cc1C]

17. The method of claim 1 wherein the immune response is an antibody response.

18. The method of claim 1 wherein the immune response is a T cell response.

19. The method of claim 1 wherein at least one of the priming and boosting immunogenic compositions comprises an adjuvant.

20. The method of claim 1 wherein at least one of the priming and boosting immunogenic compositions is administered by a route selected from buccal, inhalation, intramuscular, intranasal, intraperitoneal, intrathecal, intravenous, oral, rectal, sublingual, transdermal, vaginal or to the interstitial space of a tissue.

21. A priming vaccine comprising an immunologically effective amount of an antigen encoded by either an adenoviral vector or an RNA molecule followed by a boosting vaccine comprising an immunologically effective amount of an antigen encoded by either an adenoviral vector or an RNA molecule for use in preventing or treating a disease caused by a pathogenic organism, wherein if the priming vaccine is encoded by an adenoviral vector, the booster vaccine is encoded by an RNA molecule, and if the priming vaccine is encoded by an RNA molecule the booster vaccine is encoded by an adenoviral vector.

22. A kit for a prime boost administration regimen according to claim 1, comprising at least two vials, the first vial containing a vaccine for the priming administration and the second vial containing a vaccine for the boosting administration.
